(19) United States
(12) Patent Application Publication
(10) Pub. No.: US 2026/0191974 A1
(43) Pub. Date: Jul. 9, 2026

(54) B7-H3 TARGETING LIGANDS AND METHODS OF USE

(71) Applicant: The University of North Carolina at Chapel Hill, Chapel Hill, NC (US)

(72) Inventors: Chad Pecot, Pittsboro, NC (US); Albert Bowers, Chapel Hill, NC (US); Jillian Perry, Durham, NC (US); Matthew Bowler, Carrboro, NC (US)

(21) Appl. No.: 19/131,682

(22) PCT Filed: Nov. 22, 2023
(86) PCT No.: PCT/US2023/080897

§ 371 Date: May 21, 2025

Related U.S. Application Data
(60) Provisional application No. 63/384,889, filed on Nov. 23, 2022.

(51) Int. Cl. A61K 47/64; C07K 7/08; C07K 7/56

(52) U.S. Cl. CPC A61K 47/64; C07K 7/08; C07K 7/56

(57) ABSTRACT

Disclosed herein are B7-H3 targeting ligand peptides which may be linear or cyclized. The B7-H3 targeting ligand peptides can be associated with a cargo, optionally via a linker. Compositions comprising the B7-H3 targeting ligand peptides and their cargo can be used in methods of targeting cells comprising the B7-H3 receptor. Methods of treating or preventing diseases associated with cells comprising the B7-H3 receptor are also provided.

### STATEMENT OF PRIORITY

This application claims the benefit, under 35 U.S.C. § 119(e), of U.S. Provisional Application No. 63/384,889, filed Nov. 23, 2022, the entire contents of which are incorporated by reference herein.

### STATEMENT OF GOVERNMENT SUPPORT

This invention was made with government support under Grant Number GM125005 awarded by the National Institutes of Health. The government has certain rights in the invention.

### STATEMENT REGARDING ELECTRONIC FILING OF A SEQUENCE LISTING

A Sequence Listing in XML format, entitled 5470-936WO_ST26.xml, 307,701 bytes in size, generated on Nov. 21, 2023, and filed herewith, is hereby incorporated by reference in its entirety for its disclosures.

### FIELD OF THE INVENTION

The invention relates to B7-H3 targeting ligand peptides. The invention further relates to methods of using the targeting ligands to target cargo to a cell that comprises one or more B7-H3 receptors.

### BACKGROUND

Expression across multiple tumor types and restricted expression in normal tissues make B7-H3 an attractive target for cancer therapies. mRNA display is a powerful, high-throughput technology for discovering novel peptide ligands for protein targets. A number of methods have been used to expand the chemical diversity of mRNA display libraries beyond the 20 canonical amino acids, including genetic code reprogramming and biorthogonal chemistries. To date, however, there have been few reports using enzymes as biocompatible reagents for diversifying mRNA display libraries. Novel binders to cell receptors can be identified via these assays, which may augment peptide affinity by helping to pre-adopt a lower energy binding conformation and can typically make peptides more resistant to proteolysis and more cell permeable.

There is a need in the field therefore, to identify novel binders to cell receptors and to develop improved assays for their identification.

### SUMMARY OF THE INVENTION

The present invention is based, in part, on the development of B7-H3 ligands with high affinity for the receptor. These are versatile molecules that can be used to specifically direct therapeutic or diagnostic payloads to cancer cells while sparing toxicity to other normal tissues. In one embodiment, B7-H3 targeting ligand peptides are provided comprising, consisting essentially of, or consisting of an amino acid sequence of formula I:

(I)
(SEQ ID NO: 1)
WALQX₁X₂X₃X₄X₅X₆X₇X₈X₉KSG

wherein each of X₁-X₉ is any amino acid. Each of the specified amino acids in the formula encompasses an amino acid analog or mimetic of the specified amino acid.

In an aspect, the targeting ligand is according to formula I, wherein X₁ is L or an amino acid analog or mimetic thereof; X₂ is C or an amino acid analog or mimetic thereof; X₃, X₄ and X₅ are any amino acid; X₆ is F or an amino acid analog or mimetic thereof; X₇ is Y or an amino acid analog or mimetic thereof; X₈ is T or an amino acid analog or mimetic thereof; and X₉ is C or an amino acid analog or mimetic thereof. In an aspect, the targeting ligand is according to formula I, wherein X₁ is L or an amino acid analog or mimetic thereof; X₂ is C or an amino acid analog or mimetic thereof; X₃ is selected from H, V, T, S, and I or an amino acid analog or mimetic of any of these amino acids; X₄ is S; X₅ is selected from M, K, R, Nle, and Y or an amino acid analog or mimetic of any of these amino acids; X₆ is selected from F, Y, and W or an amino acid analog or mimetic thereof of any of these amino acids; X₇ is Y or an amino acid analog or mimetic thereof; X₈ is T or an amino acid analog or mimetic thereof; and X₉ is C or an amino acid analog or mimetic thereof.

In an embodiment, the targeting ligand comprises, consists essentially of, or consists of an amino acid sequence selected from: WALQLCHSMFYTCKSG (SEQ ID NO:2), WALQLCHSNleFYTCKSG (SEQ ID NO:3), WALQLCVSKYYTCKSG (SEQ ID NO:4), WALQLCTSMFYTCKSG (SEQ ID NO:5), WALQLCSSRFYTCKSG (SEQ ID NO:6), WALQLCISRYYTCKSG (SEQ ID NO:7) (SEQ ID NO:7), WALQLCTSYWYTCKSG (SEQ ID NO:8), and WALQLCTSNleFYTCKSG (SEQ ID NO:9) or a sequence at least 80% identical thereto, e.g., at least 85%, 90%, or 95% identical thereto.

In an aspect, targeting ligand is according to formula I, comprising an isopeptide bond, e.g., between a lysine and a glutamine and/or a disulfide bridge between two cysteines in the targeting ligand.

The targeting ligand may be N-terminal acetylated WALQLCHSMFYTCKSG (SEQ ID NO:10) or N-terminal acetylated WALQLCHSNleFYTCKSG (SEQ ID NO:11) or a sequence at least 80% identical thereto, e.g., at least 85%, 90%, or 95% identical thereto. In an aspect, the targeting ligand is selected from compounds 1 and 2.

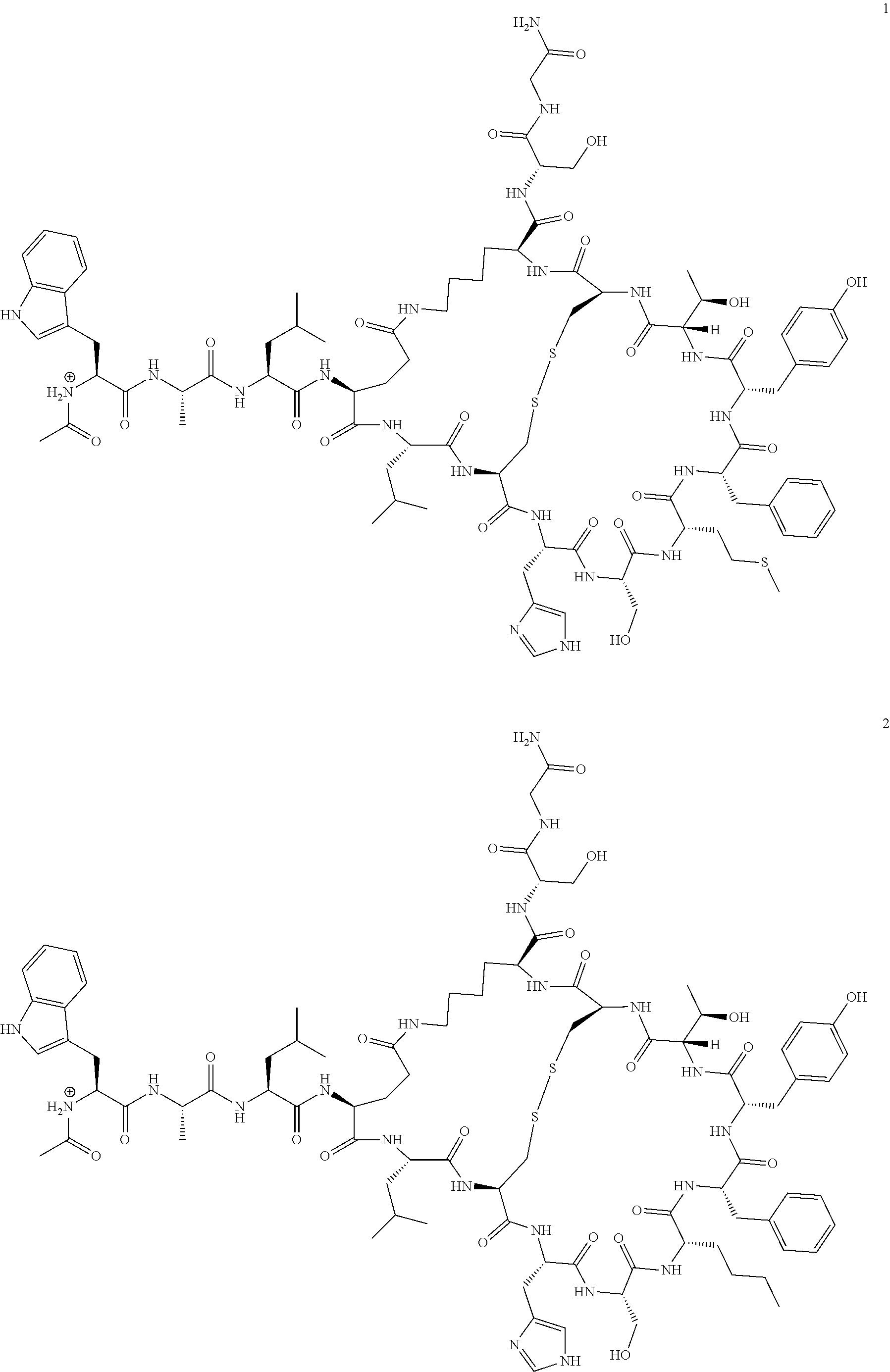

In an aspect, the targeting ligand is WALQAFTHSCIITKSG (SEQ ID NO:12), WALQYTTHSCIRPKSG (SEQ ID NO:13), or WALQYEIHSCYRNKSG (SEQ ID NO:14) or a sequence at least 80% identical thereto, e.g., at least 85%, 90%, or 95% identical thereto.

In one embodiment, B7-H3 targeting ligand peptides are provided comprising, consisting essentially of, or consisting of an amino acid sequence of formula II.

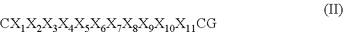

wherein each of X₁-X₉ is any amino acid and X₁₀ and X₁₁ are each independently any amino acid or absent. Each of the specified amino acids in the formula encompasses an amino acid analog or mimetic of the specified amino acid.

In an aspect, the targeting ligand is according to formula II, wherein X₁ is selected from V, I, F, and W or an amino acid analog or mimetic of any of these amino acids; X₂ is selected from V, T, I, K, V, and L or an amino acid analog or mimetic of any of these amino acids; X₃ is selected from W, I, R, F, V, and Y or an amino acid analog or mimetic of any of these amino acids; X₄ is selected from T, R, D, W, N, T, and K or an amino acid analog or mimetic of any of these amino acids; X₅ is selected from R, D, T, D, A, N, R, and G or an amino acid analog or mimetic of any of these amino acids; X₆ is selected from I, Y, D, L, P, I, R, and G or an amino acid analog or mimetic of any of these amino acids; X₇ is selected from P, N, Y, A, I, F, P, F, and K or an amino acid analog or mimetic of any of these amino acids; X₈ is selected from R, G, T, P, N, S, L, and A or an amino acid analog or mimetic of any of these amino acids; X₉ is selected from F, T, R, Y, I, and L or an amino acid analog or mimetic of any of these amino acids; X₁₀ is absent, L, R, T, V, W, Y, or I or an amino acid analog or mimetic of any of these amino acids; and X₁₁ is absent, P, L, or V or an amino acid analog or mimetic of any of these amino acids; or a sequence at least 80% identical thereto, e.g., at least 85%, 90%, or 95% identical thereto. Substitutions of the amino acids can be made, and may be according to desired function, including, for example, charge, aromaticity, and R group functionality, as detailed elsewhere herein.

In an aspect, the targeting ligand according to formula II is an amino acid sequence selected from: CIVWTRIPRFLPCG (SEQ ID NO:15), CWTIRDINGTRCG (SEQ ID NO:16), CVIRDTYYTFTCG (SEQ ID NO:17), CVIFWDDAPRVLCG (SEQ ID NO:18), CVKVNALINYWVCG (SEQ ID NO:19), CVVITNPFSIYCG (SEQ ID NO:20), CFLYKGRFLLCG (SEQ ID NO:21), and CILWNRGKAYICG (SEQ ID NO:22) or a sequence at least 80% identical thereto, e.g., at least 85%, 90%, or 95% identical thereto. In an embodiment, the targeting ligand is selected from N-terminal acetylated CIVWTRIPRFLPCG (SEQ ID NO:23) and N-terminal acetylated CWTIRDINGTRCG (SEQ ID NO:24) or a sequence at least 80% identical thereto, e.g., at least 85%, 90%, or 95% identical thereto, comprising a thioether bond between the two cysteines of the targeting ligand. In an aspect, the targeting ligand is selected from compounds 3 and 4.

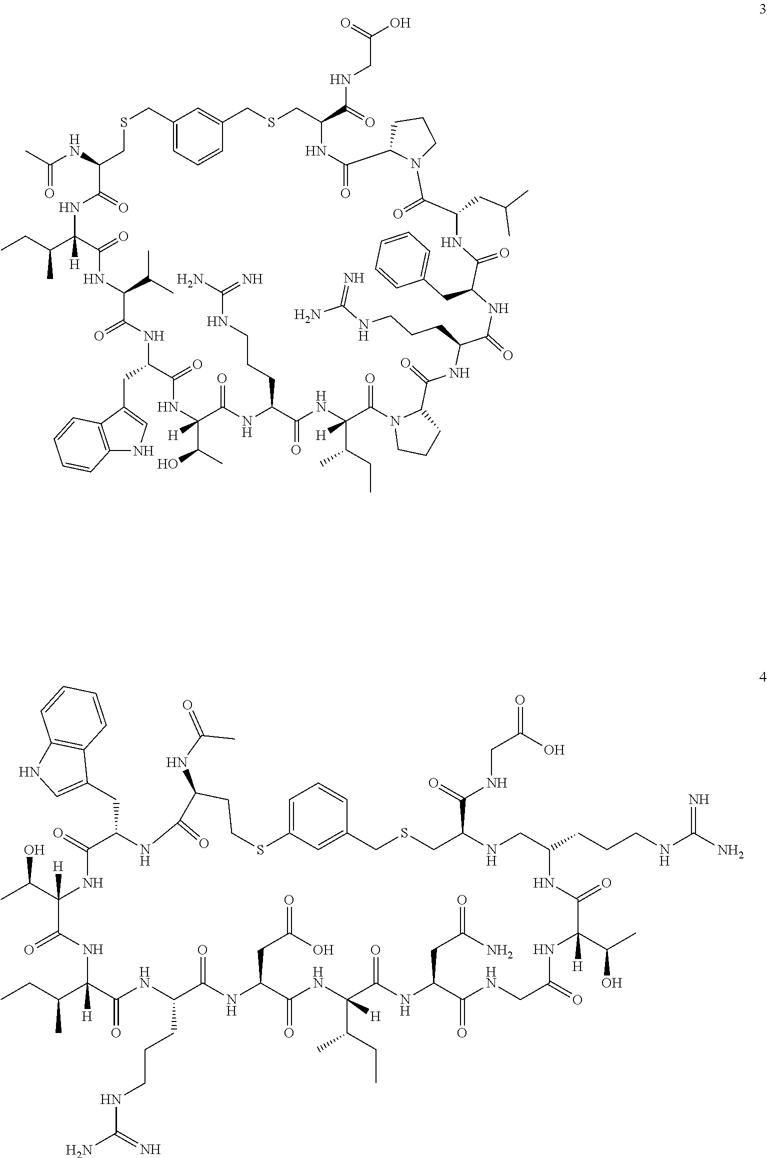

The targeting ligands disclosed herein may comprise a cap on the N-terminus of the amino acid, optionally the N-terminus is acetylated.

The targeting ligands disclosed herein can further comprise a cargo associated with the ligand. In some embodiments, the cargo can be associated by a covalent bond at the N terminus or C terminus of the targeting ligand. In an aspect, the cargo is associated with the targeting ligand via a cleavable linker. The cargo can comprise, e.g., a detectable label, biologically active agent, imaging agent, and/or therapeutic agent. In embodiments, the cargo comprises a nucleic acid, a protein, a complex of a nucleic acid and a protein, a carbohydrate, a lipid, or a small molecule.

Compositions comprising the targeting ligands are also provided. In some embodiments, a pharmaceutical composition comprising the targeting ligand disclosed herein and a pharmaceutically acceptable carrier is provided.

Methods of targeting a cargo to a cell that comprises one or more B7-H3 receptors, comprising contacting the cell with the B7-H3 targeting ligand are disclosed herein. In an aspect, the cell is a cancer cell. In some embodiments, the cancer cell may be a lung cell or epithelial cell.

Methods of treating a disease or disorder involving a cell comprising B7-H3 in a subject in need thereof, the method comprising administering to the subject a therapeutically effective amount of a targeting ligand disclosed herein comprising a cargo, thereby treating the disease or disorder are disclosed herein. In an aspect, the disease is cancer.

Methods of delivering a targeting ligand to a cell comprising B7-H3 in a subject, the method comprising administering to the subject a targeting ligand disclosed herein comprising a cargo are disclosed herein. In an aspect, the delivery is to a cancer cell, e.g., a lung cell or epithelial cell. In an aspect, the delivery is to a subject that has, or is suspected of having, cancer, and the targeting ligand comprises a cargo.

These and other aspects of the invention are set forth in more detail in the description of the invention below.

### BRIEF DESCRIPTION OF THE DRAWINGS

FIGS. 1A-1C. (FIG. 1A) The reaction catalyzed by microbial transglutaminase (mTG) to form an isopeptidic bond between a glutamine substrate and a primary amine. This reaction can be carried out in an inter or intramolecular fashion. (FIG. 1B) The glutamine activation assay. Peptide-RNA fusions containing a fixed glutamine residue can be modified by reacting a primary amine containing ligand with mTG. This will enable functionalization of the fusion and facilitate isolation assays. (FIG. 1C) The lysine cyclization assay. mTG can be used to install a macrocycle on fusions containing both lysine and glutamine residues. This will enable affinity selection assays against targets of interest using enzymatically modified libraries.

FIGS. 2A-2F. (FIG. 2A) Schematic detailing the process for assessing substrate bias during glutamine activation. (FIG. 2B) Table detailing library used and Next Generation Sequencing (NGS) statistics for the assay. The diversity is calculated as 19ⁿ where n is equal to the number of randomized positions. All 20 canonical amino acids were used in this analysis. (FIG. 2C) qPCR results of streptavidin captured material. Filled bars show recoveries after mTG treatment while open bars show recoveries if the library is not treated with mTG. Values are normalized to input samples for each replicate. This isolated material was sent for NGS analysis. (FIG. 2D) NGS results for average amino acid positional variation at each randomized position of sequences isolated from the glutamine activation assay. (FIG. 2E) Bar graph depicting the NGS results of isolated fusions from ligation assay. (FIG. 2F) Individual replicates for NGS ligation data. Values>1 indicate amino acids enriched while values<1 denote a loss of amino acids at each randomized position, N=3. Center line designates the position of the fixed glutamine residue. Charged amino acids include R, H, and K; neutral amino acids include C, S, T, and M; negative amino acids include N, Q, D and E; aliphatic amino acids include A, V, L and I; aromatic amino acids include F, Y and W; and unique amino acids include G and P.

FIGS. 3A-3K. (FIG. 3A) Schematic detailing the process for assessing substrate bias during lysine cyclization. Note, it is possible for trypsin to cleave within the ring of the peptide but the isopeptidic bond maintains the connection between the biotin handle and mRNA tag still enabling isolation and analysis. (FIG. 3B) In vitro translation (IVT) analysis of mTG macrocyclase activity. Top spectra depict the linear control peptide without incubation with mTG. Expected mass is 1452.716 daltons. Bottom spectra demonstrate the modification after incubation with mTG. Expected mass is 1435.690 daltons. (FIG. 3C) Table detailing libraries used and NGS statistics for the assay. The diversity is calculated as 19ⁿ where n is equal to the number of randomized positions. Methionine is left out to enable the translation of a biotin handle at the first amino acid position in each fusion. (FIG. 3D) Cyclization/trypsin assay on the control sequence. Fusions were treated with increasing amounts of trypsin (0 to 800 nM). (FIG. 3E) Time course recovery assay with the NNK₄ library over 1 h mTG treatment. (FIG. 3F) Cyclization/trypsin assay on both NNK₄ and NNK₆ libraries after 1 h mTG treatment. The isolated material was sent for NGS analysis. Filled bars show recoveries after trypsin digestion of (+) mTG while open bars show recoveries if the library is not treated with mTG. All values for FIG. 3D-3F are normalized to a non-digested control. (FIG. 3G) Amino acid-level analysis of NNK₄ and NNK₆ libraries. (FIG. 3H) Averaged NNK₄ lysine cyclization NGS data. Dashed lines indicate the average standard deviation of all NGS values. (FIG. 3I) Individual replicates of NNK₄ lysine cyclization NGS data. (FIG. 3J) Averaged NNK₆ NGS of (+) mTG isolated material. (FIG. 3K) Individual replicates for NNK₆ NGS ligation data. Values>1 indicate amino acids enriched while values<1 denote a loss of amino acids at each randomized position, N=3. The position of the fixed glutamine and lysine residues are indicated. Charged amino acids include R, H, and K; neutral amino acids include C, S and T; negative amino acids include N, Q, D and E; aliphatic amino acids include A, V, L and I; aromatic amino acids include F, Y and W; and unique amino acids include G and P.

FIGS. 4A-4G. (FIG. 4A) Selection schematic detailing the selection process for identifying binders against B7-H3. (FIG. 4B) qPCR results of the mTG mediated B7-H3 selection. (FIG. 4C) B7-H3 selection results and complete NGS analysis of B7-H3 hits. (FIG. 4D) NGS data of the selection hits. One family type was observed for this selection. Arrow indicates the peptide pursued for binding validation. (FIG. 4E) EIC ([M+2], 948.91 m/z) traces corresponding to the product of the mTG reaction with linear B1.1 peptide and the synthetic lactam, disulfide peptide. (FIG. 4F) Surface Plasmon Resonance (SPR) analysis of the selected peptide (B1.1), the Met9Nle variant, and the linear variant. Top shows the fitted curves for each peptide. The selected B1.1 and the Met9Nle variant were analyzed at analyte concentrations of 7.5, 15, 30, 60, 120 nM. The linear variant was analyzed at analyte concentrations of 50, 100, 200, 400, 800 nM. Bottom shows binding kinetics of each peptide. (FIG. 4G) Individual SPR curves. Fitted curve and raw data are shown.

FIG. 5. Schematic of B7-H3-targeting ligand comprising an exemplary oligonucleotide therapeutic for cargo delivery to solid tumors. B7-H3-targeting ligands may include B7-H3 linear and cyclic targeting ligands. Schematic of receptor types is shown.

FIG. 6. B7-H3 receptor distribution. Shown is a heatmap of receptor expression in myeloma and carcinoma cell lines.

FIGS. 7A-7C. mRNA display schematic and cyclization methods. (FIG. 7A) General mRNA display schematic. (FIG. 7B) Depiction of microbial transglutaminase (mTG)-mediated cyclization. (FIG. 7C) Depiction of dibromoxylene-mediated thioether cyclization.

FIGS. 8A-8B. Library design for selections against B7-H3. Amino acid sequence for mTG libraries screened against B7-H3. (FIG. 8A) Libraries of 6 and 9 randomized amino acid residues were generated and screened. The intended macrocycle is shown (MTG linkage). (FIG. 8B) Amino acid sequence for DBX libraries screened against B7-H3. Libraries of 9, 10, 11 randomized amino acid residues were generated and screened. The intended macrocycle is shown (DBX linkage).

FIGS. 9A-9B. (FIG. 9A) Exemplary B7-H3 target binding structures with general sequence ac-WALQLCXXXF₁₀YTCKSG (SEQ ID NO:25) resulting from the selection process. Amino acids 7-9 display the most variability in the sequencing results. The two hits validated for binding by SPR (Selected B1.1 and Met9Nle) are shown. (FIG. 9B) Potential cargo attachment points for the targeting ligand peptide B1.1 Met9Nle shown with arrows.

FIGS. 10A-10C. Linear B7-H3 selection with mTG-NNK₉ library. (FIG. 10A) The selection results for the linear library against B7-H3. (FIG. 10B) Relevant NGS hits from the selection. (FIG. 10C) Sequence validation assay for Lin B2.1.

FIGS. 11A-11F. Dibromoxylene (DBX) mediated B7-H3 selection. (FIG. 11A) Selection schematic details the selection process for identifying binders against B7-H3 using DBX. (FIG. 11B) qPCR results of the DBX mediated B7-H3 selection. (FIG. 11C) Mean frequency vs. number of sequences. (FIG. 11D) NGS data of the selection hits from Selections (rounds 3 and 5), named sequences indicate sequences purchased for binding validation. (FIG. 11E) Validation assay identified hits from NGS data run through the selection process (outlined in (FIG. 11A)), with the naïve NNK10 library as a control. JP3 and JP5 were identified as strong B7-H3 binders.

FIG. 12. Exemplary B7-H3 targeting ligand structures and cargo attachment points discovered via the DBX-mediated B7-H3 selection.

FIGS. 13A-13C. JP5 single variant library results. (FIG. 13A) Potential library members with the parent JP5 sequence on the top. Each residue was mutated to include potentially 20/20 canonical amino acids. (FIG. 13B) All library members were screened first against background components (magnetic beads) to remove nonspecific binders and then against biotinylated B7-H3. (FIG. 13C) The results were analyzed by qPCR which showed much higher recovery when the library was incubated with B7-H3.

FIGS. 14A-14B. JP3 single variant library results. (FIG. 14A) Potential library members with the parent JP3 sequence on the top. Each residue was mutated to include potentially 20/20 canonical amino acids. All library members were screened first against background components (magnetic beads) to remove nonspecific binders and then against biotinylated B7-H3 (see FIG. 13B). (FIG. 14B) The results were analyzed by qPCR which showed much higher recovery when the library was incubated with B7-H3.

FIG. 15. JP5 single variant library NGS dataset. Horizontal values show the parent/identified JP5 sequence (WTIRDINGTR; SEQ ID NO:197) and the effect that mutating each residue individually to 1 of the 20 canonical has on display recovery against B7-H3 (in box below each residue). Recovery values are demonstrated on the vertical axis as a fold-change. Mutations that recovery better than the parent JP5 recovery with values>1 whereas <1 indicates a hindrance on interaction. Each bar represents an amino acid. The amino acids are arranged by type and are, in order, R, H, K, C, S, T, M, N, Q, D, E, A, V, L, I, F, Y, W, G, and P.

FIG. 16. JP3 single variant library NGS dataset. Horizontal values show the parent/identified JP5 sequence (IVWTRIPRFLP; SEQ ID NO:168) and the effect that mutating each residue individually to 1 of the 20 canonical has on display recovery against B7-H3 (in box below each residue). Recovery values are demonstrated on the vertical axis as a fold-change. Mutations that recovery better than the parent JP3 recovery with values>1 whereas <1 indicates a hindrance on interaction. Each bar represents an amino acid. The amino acids are arranged by type and are, in order, R, H, K, C, S, T, M, N, Q, D, E, A, V, L, I, F, Y, W, G, and P.

FIGS. 17A-17B. JP5 alanine mutant library design and screen. (FIG. 17A) Each library member with the parent JP5 sequence on the top. JP5 was mutated at each residue to an Alanine. All library members, and JP5, were screened first against background components (magnetic beads) to remove nonspecific binders and then against biotinylated B7-H3 (see FIG. 14B). (FIG. 17B) The results were analyzed by NGS which showed relative enrichment values for each Alanine Mutant (AM). A value of >1 indicates stronger enrichment in the selection, whereas a value<1 indicates a stronger enrichment in the input library.

FIGS. 18A-18B. Serum stability of JP3 (FIG. 18A) and JP5 (FIG. 18B).

FIGS. 19A-19B. (FIG. 19A) Results of in vivo evaluation of JP3 in mice inoculated with subcutaneous A427 (lung cancer) tumors. (FIG. 19B) Exemplary targeting ligand JP3 serial dilution in H727 cells showing a dose-response by FACS for tertramethylrhodamine (TAMRA) signal.

FIG. 20 shows in vivo uptake of JP3-TAMRA and JP5-TAMRA by B7-H3 expressing H727 tumor cells 3 hours post-injection.

FIGS. 21A-21B. Live imaging of example targeting ligand uptake in Raji cells expressing murine B7-H3 for JP3 (FIG. 21A) and JP5 (FIG. 21B). 1p M peptide used with cell incubation of 2 hours.

FIGS. 22A-22B. Live imaging of an example targeting ligand uptake in Raji cells expressing murine B7-H3 pre-incubated with GFP-tagged reporters for early (Rab5a) and late (Rab7a) endosomes or early (Lamp1) lysosomes. Cells were treated with JP3-PEG-TAMRA (FIG. 22A) or JP5-PEG-TAMRA (FIG. 22B) at 250 nM for 5 hours, cytospun with live cell imaging subsequently performed.

FIGS. 23A-23C. Tissue distribution (FIGS. 23A-23B) and tumor accumulation (FIG. 23C) of JP5 was evaluated in vivo in mice with A431 tumors overexpressing B71-H13.

FIGS. 24A-24C. SPR analysis of the selected peptides B1.1 and JP5. The B1.1 variant was analyzed at analyte concentrations of 7.5, 15, 30, 60, and 120 nM (FIG. 24A) and the JP5 variant was analyzed at analyte concentrations of 22.2, 66.6, 200, 600 and 1800 nM (FIG. 24B). (FIG. 24C) Binding kinetics of each peptide.

FIGS. 25A-25D. Tissue distribution (FIGS. 25A-25B) and tumor accumulation (FIGS. 25C-25D) of B1.1 and JP5 ligands (Cy5 labeled) was evaluated. Athymic nude mice bearing A431 wild-type (overexpressing B7H3) flank tumors were treated with PBS, JP5-Cy5, or B1.1-Cy5 (15 nmol, via subcutaneous injection). 24 h post injection, mice were euthanized and tissues harvested and analyzed for fluorescent signal using the IVIS Lumina. Signal for JP5 and B1.1 was observed in the flank tumors, as well as the liver, kidney, and lungs. (FIG. 25A) Graphic shows average fluorescence signal per mg of tissue for n=3 tumors for JP5, n=4 tumors for B1.1, and n=9 somatic tissues. (FIG. 25B) Graphic displaying % recovered fluorescence for mice treated with either JP5 or B1.1. For these samples, the average background fluorescence signal (from PBS control group) for each tissue was subtracted from each individual tissue sample for the JP5 and B1.1 treatment groups. Fluorescence signal collected from each tissue for each individual mouse was summed to determine total fluorescent signal for each mouse. Each organ fluorescence signal was then divided by the total signal from the mouse to determine % recovered fluorescence for each tissue. This graphic represents the average and standard deviation for n=3 tumors for JP5, n=4 tumors for B1.1, and n=9 somatic tissues for JP5 and B1.1. (FIG. 25C) Single cell suspensions of the tumors were made following chemical and mechanical digestion, and then stained for Live/Dead dye, human anti-HLA antibody and murine anti-CD31 antibody. Represented are the percent of cancer cells (human HLA+) that took up Cy5 ligand. The p-values are 2-sided student's t-tests shown above each respective group in comparison to the PBS control group. (FIG. 25D) Single cell suspensions of the tumors were made following chemical and mechanical digestion, and then stained for Live/Dead dye, human anti-HLA antibody and murine anti-CD31 antibody. Represented are the percent of CD31+ endothelial cells (murine CD31+) that took up Cy5 ligand. The p-values are 2-sided student's t-tests shown above each respective group in comparison to the PBS control group.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, 0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide”, “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), guide RNA (gRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g., a human, non-human primate, lagomorph (e.g., rabbit), or rodent (e.g., mouse, rat). In some embodiments a human subject is a neonate, child, adult or geriatric subject. In some embodiments a human subject is at least 50, 60, 70, 80, or 90 years old.

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

When any variable (e.g., Ri) occurs more than one time in any constituent or formula for a compound, its definition at each occurrence is independent of its definition at every other occurrence. Thus, for example, if a group is shown to be substituted with one or more Ri moieties, then Ri at each occurrence is selected independently from the Markush group recited for Ri. Also, combinations of substituents and/or variables are permissible, but only if such combinations result in stable compounds within a designated atom's normal valency.

“Pharmaceutically acceptable carrier” (sometimes referred to as a “carrier”) refers to a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

The present invention relates to novel binders to the therapeutic target B7-H3. B7-H3 is a member of the B7 family, with up-regulation of inhibitory B7 molecules in the cancer microenvironment appearing highly related to the immune evasion of tumors. See, Zou et al., Nat Rev Immunol. 2008; 8:467-77. B7-H3 receptors are commonly found on cancer cells and absent from non-cancer cells. See, Yang et al. Int J Bio Sci. 2020; 16(11):1767-1773; doi: 10.7150/ijbs.41105, incorporated herein by reference, specifically Table 2 (identifying non-small cell lung cancer, cutaneous squamous cell carcinoma, pancreatic cancer, primary hepatocellular carcinoma, colorectal carcinoma, clear cell renal carcinoma, and breast cancer as comprising B7-H3 expression). The targeting ligands of the present invention can be provided with cargo and delivered to cells comprising B7-H3 receptors. Accordingly, therapeutic and diagnostic compositions and methods of use of the B7-H3 targeting ligands are provided herein.

In one embodiment, B7-H3 targeting ligand peptides are provided comprising, consisting essentially of, or consisting of an amino acid sequence of formula I:

(I)
(SEQ ID NO: 1)
WALQX₁X₂X₃X₄X₅X₆X₇X₈X₉KSG

wherein each of X₁-X₉ is independently any amino acid. Each of the specified amino acids in the formula encompasses an amino acid analog or mimetic of the specified amino acid.

An amino acid, as described herein, includes both naturally occurring and/or non-naturally occurring amino acids. Naturally occurring amino acids include those encoded by the genetic code and those amino acids that are later modified, for example, carboxyglutamate and hydroxyproline. Amino acid analogs comprise a structure similar to a naturally occurring amino acid but have modified R groups, such as norleucine or norvaline, or modified peptide backbones. Non-naturally occurring amino acids may include amino acid analogs and amino acid mimetics that function similar to naturally occurring amino acids. Exemplary amino acids, analogs and mimetics include β-alanine (β-Ala), N-α-methyl-alanine (Me-Ala), aminobutyric acid (Abu), α-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (MeIle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O2)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-C1)), 4-fluorophenylalanine (Phe(4-F)), 4-nitrophenylalanine (Phe(4-NO2)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), U-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), U-Benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), 1-amino-1-cyclohexane carboxylic acid (Acx), aminovaleric acid, beta-cyclopropyl-alanine (Cpa), propargylglycine (Prg), allylglycine (Alg), 2-amino-2-cyclohexyl-propanoic acid (2-Cha), tertbutylglycine (Tbg), vinylglycine (Vg), 1-amino-1-cyclopropane carboxylic acid (Acp), 1-amino-1-cyclopentane carboxylic acid (Acpe), alkylated 3-mercaptopropionic acid, 1-amino-1-cyclobutane carboxylic acid (Acb). D and L isomers of the naturally occurring or non-naturally occurring amino acids are contemplated within the term amino acid. In certain instances, a charged amino acid can be substituted with another charged amino acid, a neutral amino acid with another neutral amino acid. In some instances, an amino acid can be substituted with an amino acid of a different charge to modify the interactions of the peptide targeting ligand. Any specified amino acid in the sequences disclosed herein may include analogs and mimetics of that amino acid. One of skill in the art is well aware of amino acid analogs and mimetics of each of the naturally occurring amino acids.

In an aspect, the targeting ligand is according to formula I, wherein X₁ is L or an amino acid analog or mimetic thereof; X₂ is C or an amino acid analog or mimetic thereof; X₃, X₄ and X₅ are any amino acid; X₆ is F or an amino acid analog or mimetic thereof; X₇ is Y or an amino acid analog or mimetic thereof; X₈ is T or an amino acid analog or mimetic thereof; and X₉ is C or an amino acid analog or mimetic thereof. In an aspect, the targeting ligand is according to formula I, wherein X₁ is L or an amino acid analog or mimetic thereof; X₂ is C or an amino acid analog or mimetic thereof; X₃ is selected from H, V, T, S, and I or an amino acid analog or mimetic of any of these amino acids; X₄ is S or an amino acid analog or mimetic thereof; X₅ is selected from M, K, R, Nle, and Y or an amino acid analog or mimetic of any of these amino acids; X₆ is selected from F, Y, and W or an amino acid analog or mimetic of any of these amino acids; X₇ is Y or an amino acid analog or mimetic thereof; X₈ is T or an amino acid analog or mimetic thereof; and X₉ is C or an amino acid analog or mimetic thereof.

In an embodiment, the targeting ligand comprises, consists essentially of, or consists of an amino acid sequence selected from: WALQLCHSMFYTCKSG (SEQ ID NO:2), WALQLCHSNleFYTCKSG (SEQ ID NO:3), WALQLCVSKYYTCKSG (SEQ ID NO:4), WALQLCTSMFYTCKSG (SEQ ID NO:5), WALQLCSSRFYTCKSG (SEQ ID NO:6), WALQLCISRYYTCKSG (SEQ ID NO:7), WALQLCTSYWYTCKSG (SEQ ID NO:8), and WALQLCTSNleFYTCKSG (SEQ ID NO:9) or a sequence at least 80% identical thereto, e.g., at least 85%, 90%, or 95% identical thereto.

The targeting ligand can, in some embodiments, be provided as a cyclized molecule. In other embodiment, the targeting ligand is provided as a linear molecule. In an aspect, the targeting ligand is according to formula I, and comprises an isopeptide bond, e.g., between a lysine and a glutamine, and/or a disulfide bridge between two cysteines in the targeting ligand.

The targeting ligand may be N-terminal acetylated WALQLCHSMFYTCKSG (SEQ ID NO:10) or N-terminal acetylated WALQLCHSNleFYTCKSG (SEQ ID NO:11) or a sequence at least 80% identical thereto, e.g., at least 85%, 90%, or 95% identical thereto. In an aspect, the targeting ligand is selected from compounds 1 and 2.

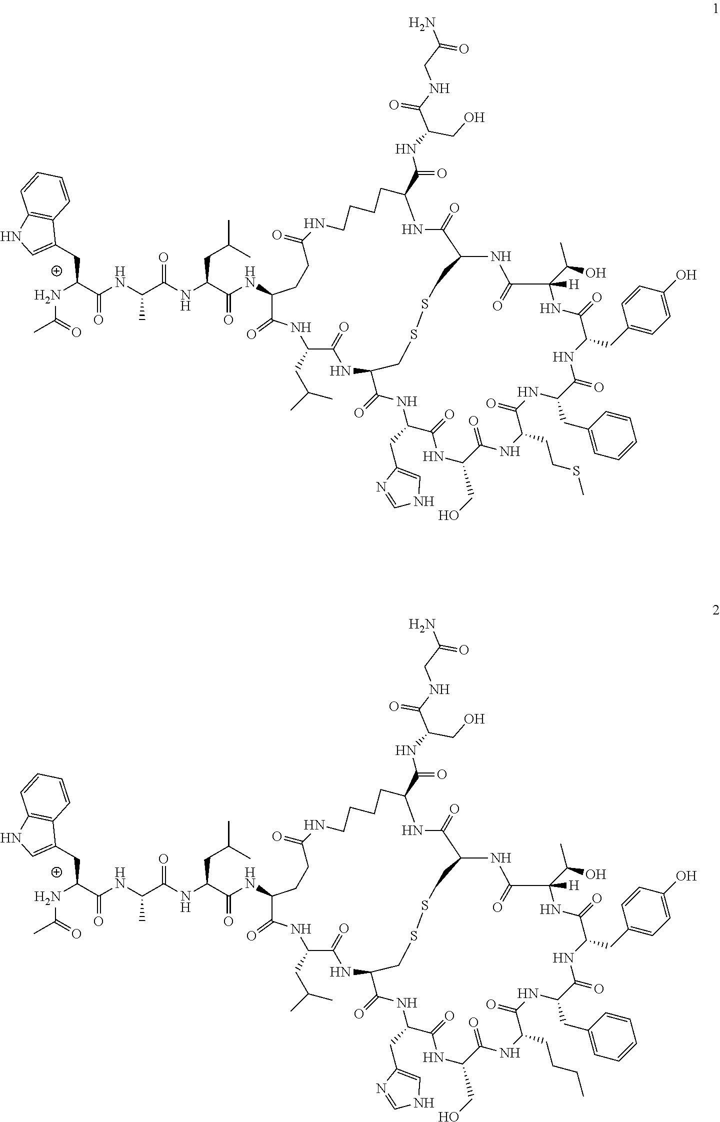

In an aspect, the targeting ligand is WALQAFTHSCIITKSG (SEQ ID NO:12), WALQYTTHSCIRPKSG (SEQ ID NO:13), or WALQYEIHSCYRNKSG (SEQ ID NO:14) or a sequence at least 80% identical thereto, e.g., at least 85%, 90%, or 95% identical thereto.

In one embodiment, B7-H3 targeting ligand peptides are provided comprising, consisting essentially of, or consisting of an amino acid sequence of formula II:

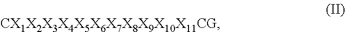

wherein each of X₁-X₉ is any amino acid and X₁₀ and X₁₁ are each independently any amino acid or absent. Each of the specified amino acids in the formula encompasses an amino acid analog or mimetic of the specified amino acid.

In an aspect, the targeting ligand is according to formula II, wherein X₁ is selected from V, I, F, and W or an amino acid analog or mimetic of any of these amino acids; X₂ is selected from V, T, I, K, V, and L or an amino acid analog or mimetic of any of these amino acids; X₃ is selected from W, I, R, F, V, and Y or an amino acid analog or mimetic of any of these amino acids; X₄ is selected from T, R, D, W, N, T, and K or an amino acid analog or mimetic of any of these amino acids; X₅ is selected from R, D, T, D, A, N, R, and G or an amino acid analog or mimetic of any of these amino acids; X₆ is selected from I, Y, D, L, P, I, R, and G or an amino acid analog or mimetic of any of these amino acids; X₇ is selected from P, N, Y, A, I, F, P, F, and K or an amino acid analog or mimetic of any of these amino acids; X₈ is selected from R, G, T, P, N, S, L, and A or an amino acid analog or mimetic of any of these amino acids; X₉ is selected from F, T, R, Y, I, and L or an amino acid analog or mimetic of any of these amino acids; X₁₀ is absent, L, R, T, V, W, Y, or I or an amino acid analog or mimetic of any of these amino acids; and X₁₁ is absent, P, L, or V or an amino acid analog or mimetic of any of these amino acids; or a sequence at least 80% identical thereto, e.g., at least 85%, 90%, or 95% identical thereto. Substitutions of the amino acids can be made, and may be according to desired function, including, for example, charge, aromaticity, and R group functionality, as detailed elsewhere herein.

In an aspect, the targeting ligand according to formula II comprises, consists essentially of, or consists of an amino acid sequence selected from: CIVWTRIPRFLPCG (SEQ ID NO:15), CWTIRDINGTRCG (SEQ ID NO:16), CVIRDTYYTFTCG (SEQ ID NO:17), CVIFWDDAPRVLCG (SEQ ID NO:18), CVKVNALINYWVCG (SEQ ID NO:19), CVVITNPFSIYCG (SEQ ID NO:20), CFLYKGRFLLCG (SEQ ID NO:21), and CILWNRGKAYICG (SEQ ID NO:22) or a sequence at least 80% identical thereto, e.g., at least 85%, 90%, or 95% identical thereto. In an embodiment, the targeting ligand is cyclized. The targeting ligand may be selected from N-terminal acetylated CIVWTRIPRFLPCG (SEQ ID NO:23) and N-terminal acetylated CWTTRDINGTRCG (SEQ ID NO:24) or a sequence at least 80% identical thereto, e.g., at least 85%, 90%, or 95% identical thereto, comprising a thioether bond between the two cysteines of the targeting ligand. In an aspect, the targeting ligand is selected from compounds 3 and 4.

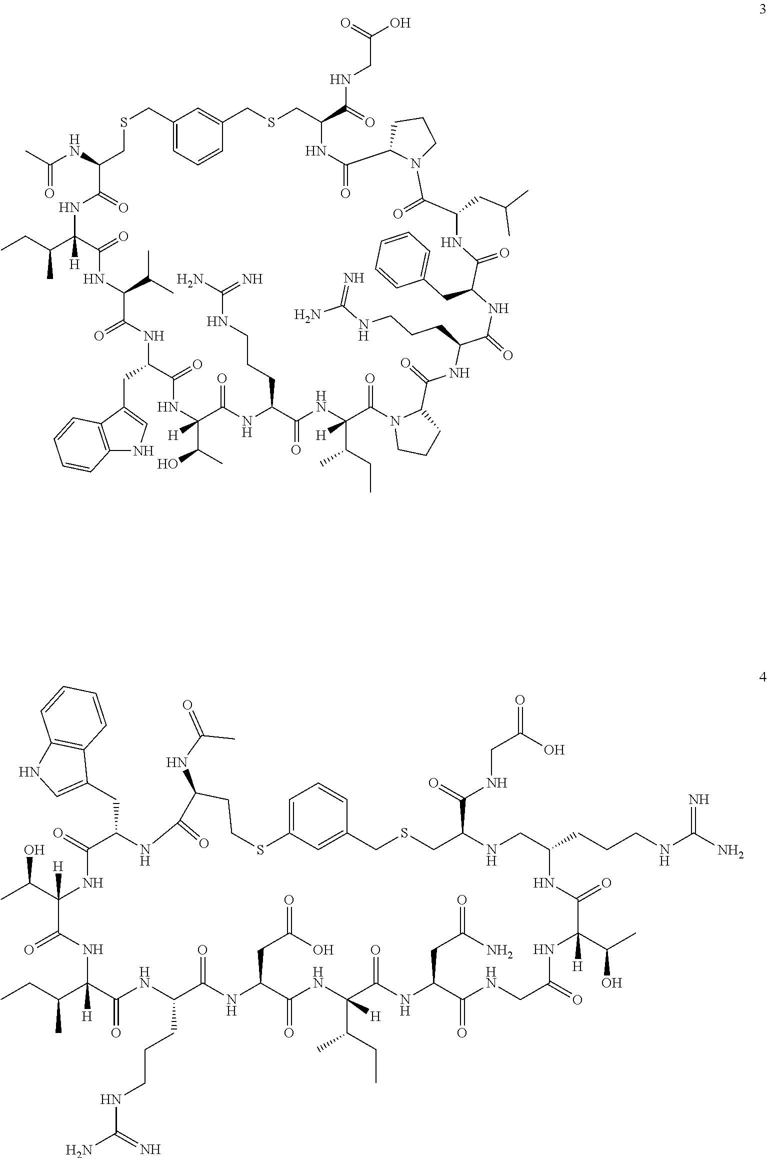

The targeting ligands disclosed herein may comprise a cap on the N-terminus of the amino acid, optionally the N-terminus is acetylated. Additional modifications of the N-terminus include formyl, pyroglutamyl, fatty acids, urea, alkylamine and sulfonamide.

The targeting ligands disclosed herein can further comprise a cargo associated with the ligand. In some embodiments, the cargo can be associated at the N terminus or C terminus of the targeting ligand, for example conjugated via covalent bonding. In an aspect, the cargo is associated with the targeting ligand via a cleavable linker, the linker covalently bonded to the targeting ligand at the N terminus or C terminus. Exemplary cleavable linkers include PEG molecules. In applications for cancer therapies, linkers can include cleavable chemical triggers including acid, GSH, phosphatase, and photoresponsive cleavable triggers. See, Su et al, Acta Pharm Sin B. 2021 December; 11(12):3889-3907; doi:10.1016/j.apsb.2021.03.042, incorporated herein by reference in its entirety, and Table 1 specifically incorporated for its disclosure of chemical trigger linkers. Linker technologies may include enzyme cleavable linkers, such as ester, amide, and carbamate, acid cleavable linkers such as hydrazone and carbonate, reducible disulfide linkers, and non-cleavable linkers such as thioether, oxime, and triazole. See, Alas, et al, “Peptide-Drug Conjugates with Different Linkers for Cancer Therapy” J Med Chem. 2021 Jan. 14; 64(1):216-232; doi: 10.1021/acs.jmedchem.Oc01530, incorporated herein by reference.

The cargo can comprise a detectable label, biologically active agent, imaging agent, and/or therapeutic agent. In embodiments, the cargo comprises a nucleic acid, a protein, a complex of a nucleic acid and a protein, a carbohydrate, a lipid, or a small molecule. The cargo can be selected for the treatment of a disease or disorder, for the detection of a disease or disorder, or for monitoring of a disease or disorder.

In an example embodiment, the cargo is an mRNA for delivery to a cell to increase expression of one or more proteins. Delivery of mRNA may be indicated when a protein is underexpressed in a cancer or other disease or disorder. In an embodiment, the cargo is an antisense oligonucleotide or siRNA for delivery to a cell to decrease expression or achieve gene silencing of one or more target sequences. The cargo may be an imaging agent or detectable label or may be a molecule comprising an imaging agent or detectable label, that can be delivered to a cell or tissue. In an aspect, the cargo is a genetic modulating agent such as a CRISPR-Cas system comprising a CRISPR-Cas protein, or a polynucleotide encoding the CRISPR-Cas protein, and a guide sequence specific for a target sequence that can be utilized for a variety of gene editing applications. Accordingly, the targeting ligands comprising cargo will find use in therapeutic, detection, diagnostic and other applications.

### Imaging Agents

Imaging agents, including reporter probes, can be delivered by the compositions, targeting ligands, and methods of the present invention. The imaging agents and reporter probes may be associated with another molecule, covalently or otherwise, for delivery by the targeting ligand. In an aspect, a reporter probe may be associated with a nucleic acid specific for a target sequence in a cell, that can be delivered to a cell, tissue, or a subject in need thereof.

Imaging agents can include molecules suitable for any imaging modality, including positron emission tomography (PET) and single-photon emission computed tomography (SPECT), magnetic resonance imaging (MRI), ultrasound (US), and Computed Tomography (CT) imaging. Example agents can include magnetic agents including paramagnetic agents, such as gadolinium, and superparamagnetic iron-based agents. See, e.g., Xiao, et al. (2016). MRI contrast agents: Classification and application. Int'l J of Mol Med, 38:1319-1326; doi:10.3892/ijmm.2016.2744. Other contrast media may include iodinated compounds, e.g., tri-iodinated benzene rings, or colloidal or micronized barium sulfate.

Reporter probes, including fluorescent, radioactive and other photo-emitting compounds, may also be delivered, including synthetic dye families such as tetramethylindo(di)-carbocyanines, fluorescein, and rhodamines. Biosensors may also be used, including in in vitro detection applications for bacterial, viral and other clinical applications. See, e.g., Castillo-Henriquez et al., Sensors (Basel). 2020 December; 20(23):6926; doi: 10.3390/s20236926.

### Biologically Active Agents

A biologically active agent, i.e., an agent that modulates an effect or activity in a cell, tissue, organ, or other biological media such as biological fluid, includes nucleic acids, proteins, small molecules, carbohydrates, lipids and complexes, salts thereof, and combinations thereof. The biologically active agent can be a genetic modifying agent. Representative nucleic acids include DNA, RNA, transposon DNA, antisense nucleic acids, ribozymes, plasmids, expression constructs, and RNA, such as mRNA, guide RNA (gRNA), tRNA, ribosomal RNA, small nucleolar RNA, antisense oligonucleotides, or RNAi, such as siRNA, shRNA, and miRNA.

RNAi therapeutic agents comprise a polynucleotide that is complementary to a portion of the target sequence mRNA. In an example embodiment, the siRNA is a nucleic acid that can form a double stranded RNA with the ability to reduce or inhibit expression of a gene or target gene: each complementary sequence of the double stranded siRNA is about 15-50 nucleotides in length, and the double stranded siRNA is about 15-50 base pairs in length. A small hairpin RNA (shRNA) is also contemplated for use. The shRNA is an antisense strand of about 19 to about 25 nucleotides followed by a short nucleotide loop (approximately 5 to 9 nt) followed by the analogous sense strand. In an embodiment, an RNAi is a microRNA or miRNA, endogenous RNAs, some of which are known to regulate the expression of protein-coding genes at the posttranscriptional level. See, e.g., Lim et al Science 299, 1540 (2003), Lee & Ambros, Science, 294:862 (2001), Lau et al., Science 294:858-861 (2001), Lagos-Quintana et al, RNA, 9:175-179 (2003).

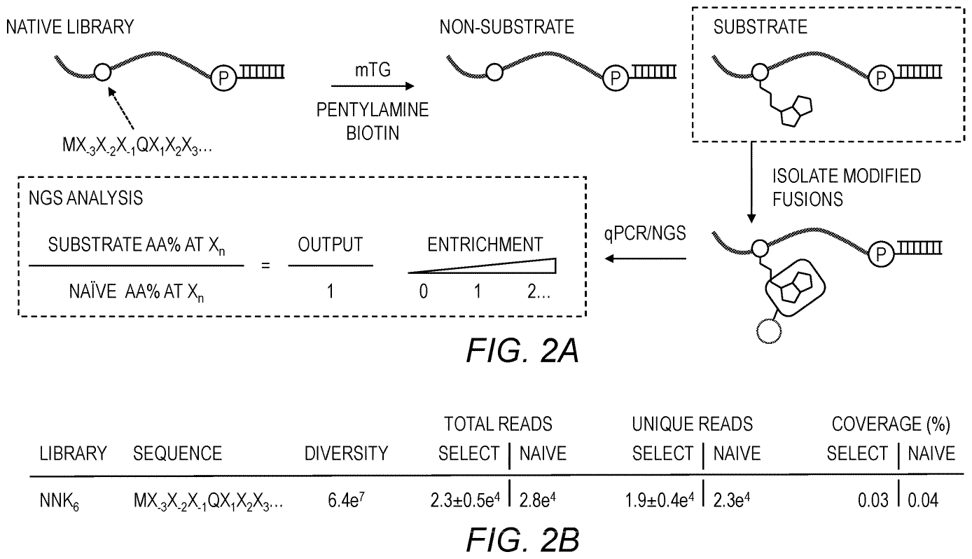

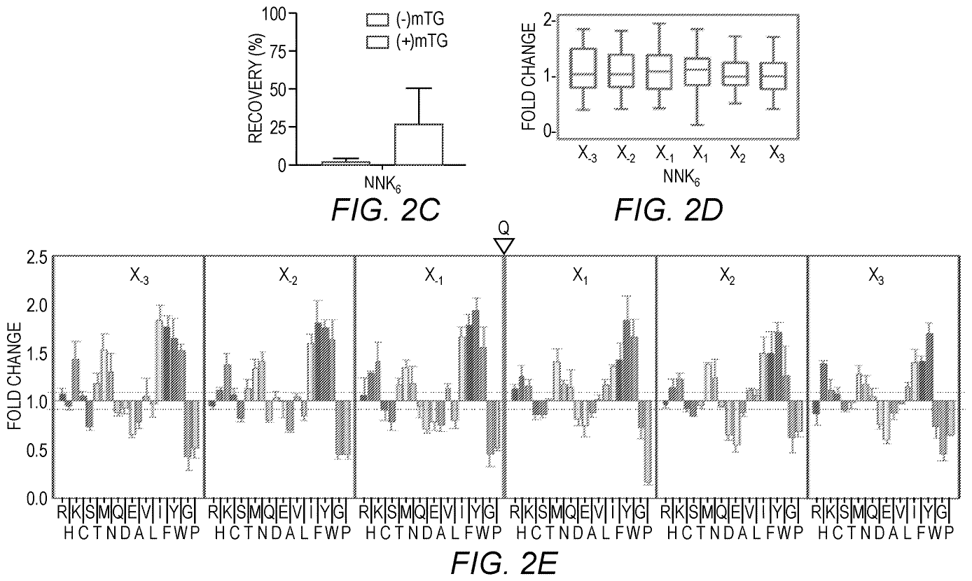

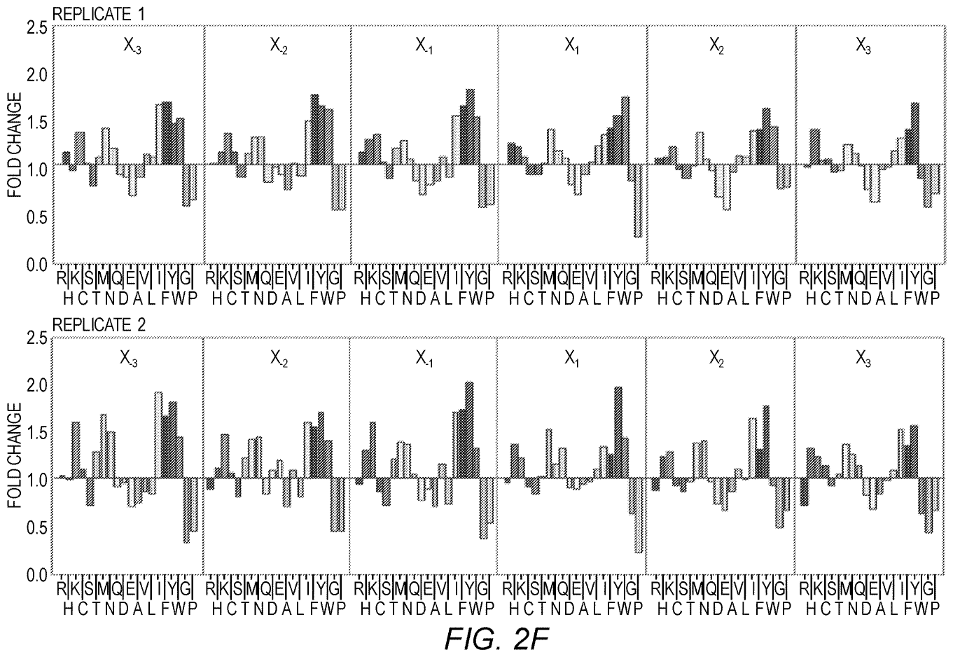

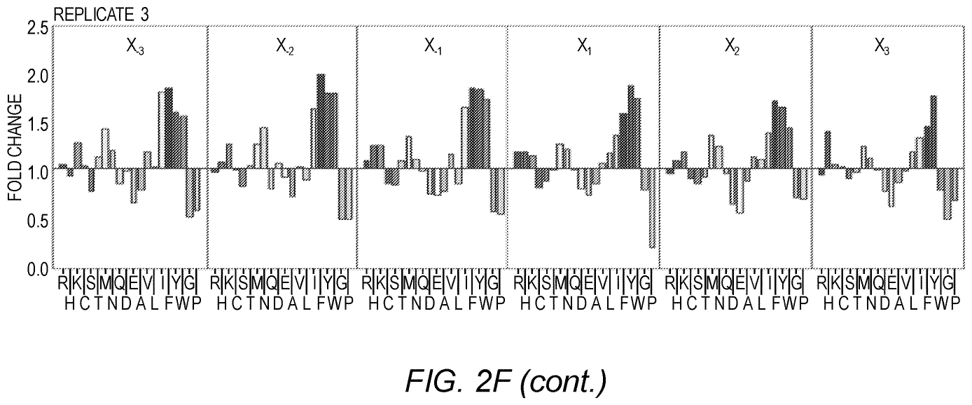

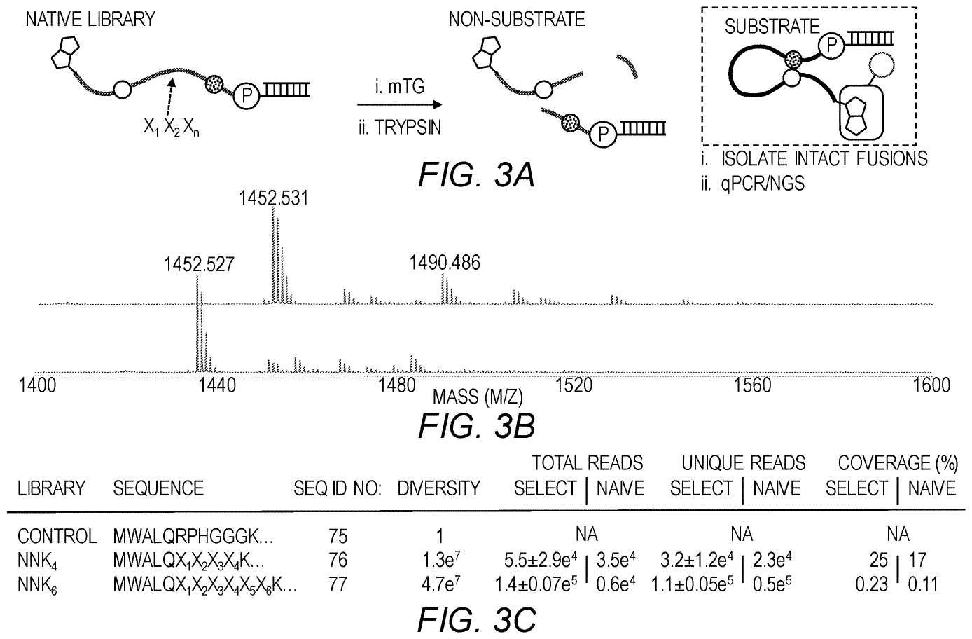

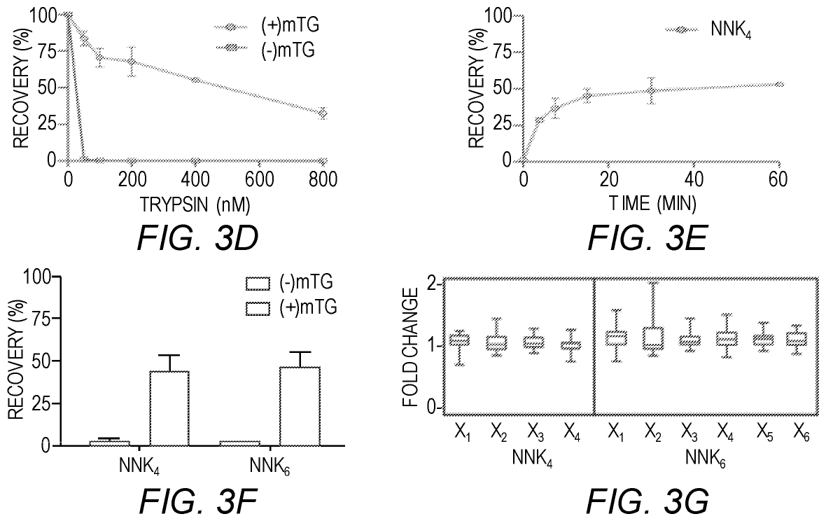

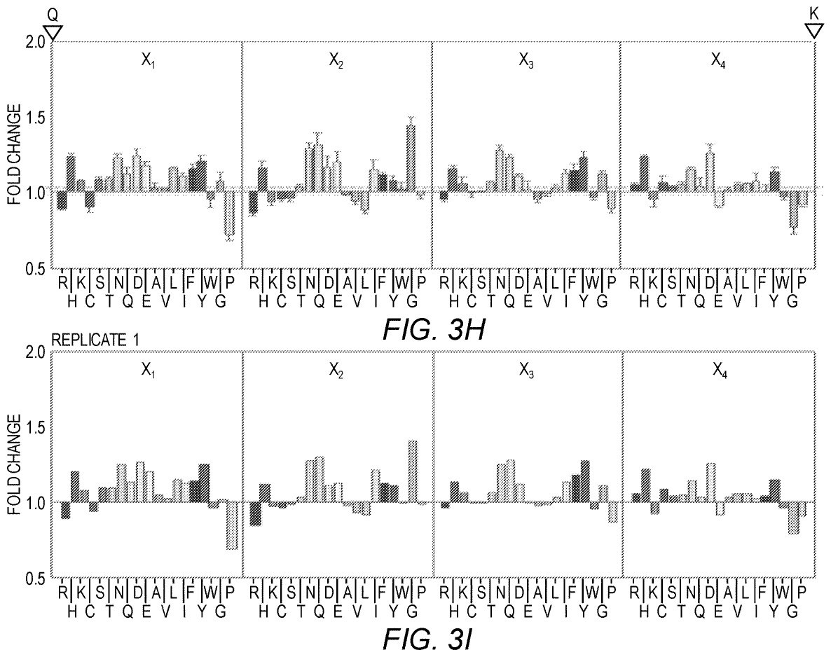

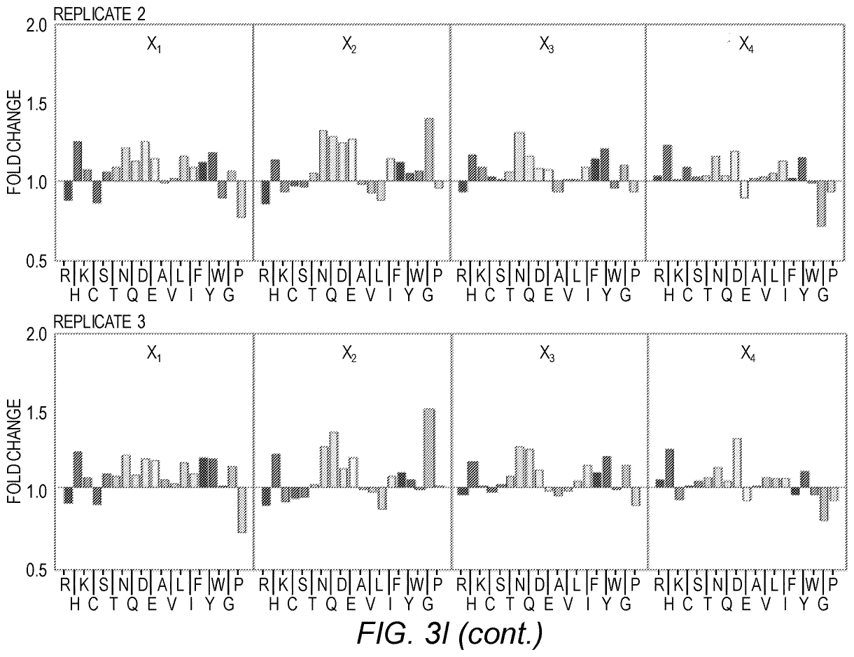

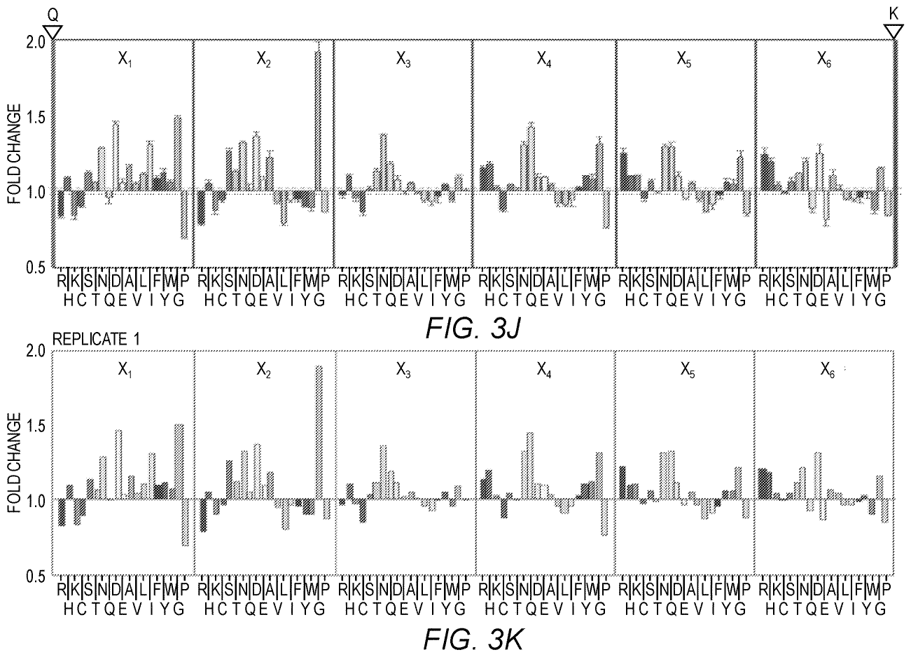

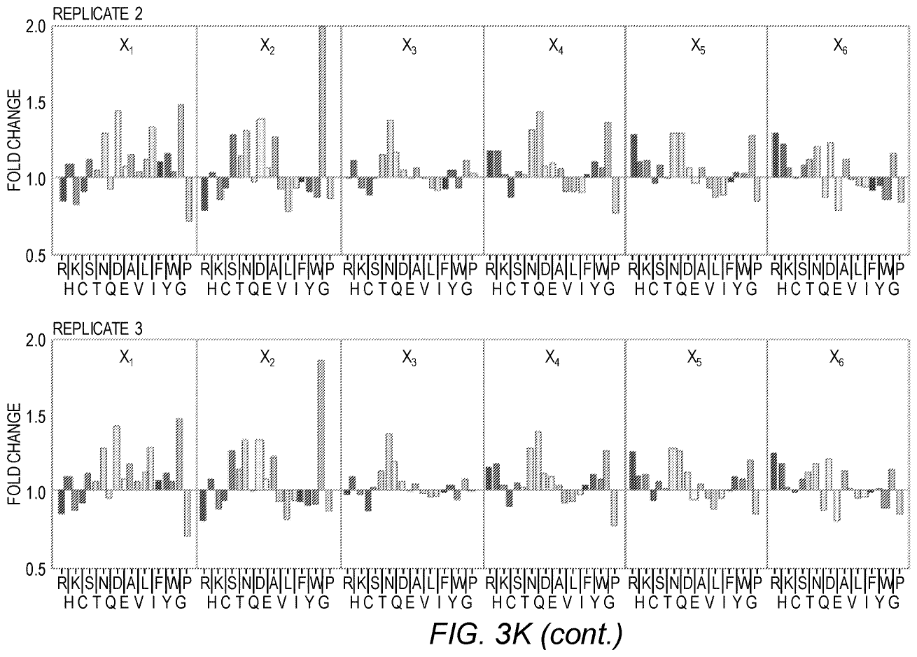

Different criteria are available for selecting the nucleic acid for use and may comprise scanning the mRNA sequence of the target, and may include empiric determination in accordance with, for example, Sui G et al., Proc. Natl. Acad. Sci. USA 99:5515-20 (2002) and may include confirmation the sequence lacks significant sequence homology with other genes as analyzed by BLAST search. Additional approaches may comprise any accessible site in endogenous mRNA that can be targeted for degradation by synthetic oligodeoxyribonucleotide/RNase H method (see, e.g., Lee N S et al., Nature Biotechnol. 20:500-05 (2002)). RNAi treatment may comprise miRNA or siRNA, or a pre-miRNA which is processed by Dicer to form a miRNA. The RNAi may also comprise a dsRNA or shRNA which is processed by Dicer to form a siRNA. The polynucleotides may comprise one or more modifications to suppress innate immune activation, enhance activity and specificity, and reduce off-target induced toxicity. Example teachings can be found, for example at Provost et al., EMBO J., 2002 Nov. 1; 21(21):5864-5874; Tabara et al., Cell 2002, June 28; 109(7):861-71; Martinez et al., Cell 2002 Sep. 6; 110(5):563; Hutvagner & Zamore, Science 2002, 297:2056. In certain embodiments, a single-stranded RNAi agent disclosed herein can comprise substitutions, or modifications, including chemically modified nucleotides, and non-nucleotides which may include incorporation in the backbone, sugars, bases, or nucleosides. In an example, siRNA may comprise dual ribose modifications, including 2′,4′- and 2′,5′-modifications, 5′-E/Z-vinylphosphonate, and northern methanocarbacyclic (NMC) modifications. See, Gangopadhyay, RNA Biol. 2022 January; 19(1):452-467; doi: 10.1080/15476286.2022.2052641. The use of substituted or modified single-stranded RNAi agents can be designed to have an increased half-life in a subject. Furthermore, certain substitutions or modifications can be used to improve the bioavailability of single-stranded RNAi agents by targeting particular cells or tissues or improving cellular uptake of the single-stranded RNAi agents. Exemplary modifications and locations within a RNAi polynucleotide are described in Hu et al. “Therapeutic siRNA: State of the Art” Signal Transduction and Targeted Therapy 5, Article number 100 (2020), incorporated herein by reference, see, e.g., FIGS. 2 and 3, specifically for its teachings of modifications.

The RNAi molecule may decrease the mRNA level in a cell for a target gene by at least about 5%, about 10%, about 20%, about 30%, about 40%, about 50%, about 60%, about 70%, about 80%, about 90%, about 95%, about 99%, or about 100% of the mRNA level found in the cell without the presence of the miRNA or RNA interference molecule

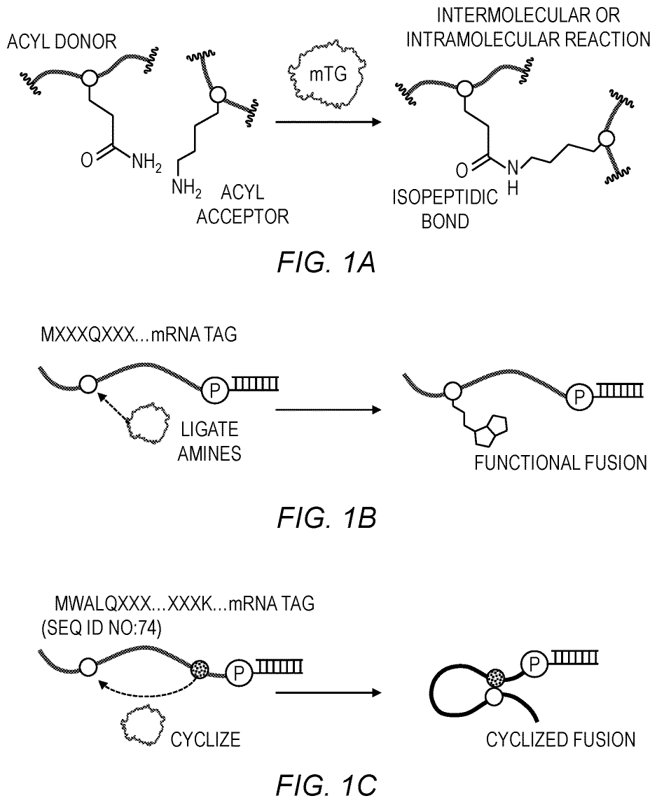

The cargo may include a gene modulating agent, including gene editing systems, or a component thereof. Gene editing systems may comprise a CRISPR system, a zinc finger nuclease system, a meganuclease, or a TALE system. A CRISPR-Cas system can comprise a Class 1 or Class 2 CRISPR-Cas system, which may comprise a guide sequence engineered to specifically bind a polynucleotide of interest. A polynucleotide encoding the CRISPR-Cas polypeptide, a guide sequence designed to complex with the CRISPR-Cas polypeptide at a target of interest, or both components, can be utilized as cargo, delivered by the same targeting ligand or different targeting ligands. As such, a ribonucleoprotein comprised of a Cas protein and a guide polynucleotide can be delivered. The CRISPR-Cas system that can be used to modify a target polynucleotide of the present invention described herein can be a Class 1 CRISPR-Cas system. Class 1 CRISPR-Cas systems are divided into types I, II, and IV. Makarova et al. 2020. Nat. Rev. 18:67-83., particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2; Type V systems include V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type VI systems include VI-A, VI-B1, VI-B2, VI-C, and VI-D. Design of guides for targeting a nucleic acid for modification is known in the art, see, e.g., IDTdna.com and Synthego.com for guidance on custom guide RNAs. Reduction of off-target effects can be tailored using programs such as GUIDE-seq for the design of guide sequences for a desired target. See, e.g., Malinin, et al., Nature Protocols 16:5592-5615 (2012).

TALEN based gene editing is also contemplated and can be used in in vivo and in vitro applications. See, S Becker, J Boch—Gene and Genome Editing, 2021. Zinc finger nuclease editing can also be utilized, and further modified to ensure high-precision gene editing. See, e.g., Conway et al., Molecular Therapy 27:4, 10 Apr. 2019, Pages 866-877; Paschon et al. Nature Comm. 10:1133 (2019). Similarly, editing can be made by meganucleases, characterized by a large recognition site of 12 to 40 based pairs of a double-stranded DNA sequence. See, e.g., U.S. Pat. Nos. 8,119,381 and 10,273,524. Gene editing tools are well known in the art, with advantages and comparison of the tools that can be considered for the desired application. Rahim et al., Int'l J. of Innovative Science and Research Tech. 6:8 (2021), incorporated herein by reference.

Transposases may be used with the methods of the present invention. Transposases include those comprising RNase H-like nuclease domains, such as Tn5, MuA, Mos1, Hermes, Serine and Tyrosine recombinases, including CTnDOT, Tn916, IS607 and TnpX, transposases comprising an HUH domain, including TnpA of IS91 or ISHp608, and helitron transposases, which can be as detailed in International Patent Publication WO 2022/056309, page 26, line 26 to page 27, line 17, specifically incorporated by reference. See also nuclease guided transposase as described in WO 2022/150651 (DNA nuclease guided Transposases systems, Tn7-like transposition proteins with a Cas12k protein), WO 2022/147321 (Type I-B CRISPR Associated Transposase systems), WO 2022/076830 (Type I CRISPR Associated transposase systems), WO 2021/257997 (CAST); Li, et al., Int. J. Mol. Sci. 2020, 21(21):8329; doi: 10.3390/ijms21218329 (Tn5 transposase in applied genomic research).

### Therapeutic Agents

Therapeutic agents that can be used as cargo conjugated to the targeting ligands can comprise modulating agents, for example, small molecules such as chemotherapeutic agents, anti-oncogenic agents, anti-microbial agents, peptides, proteins (enzymes, antibodies, peptidic hormones), non-peptidic hormones, other pharmaceutically active substances, and the like.

Examples of the protein or peptide can include hormones such as growth hormones, growth factors, cytokines, tumor necrosis factors, and growth hormone releasing factors. Example enzymes can include transferases, hydrolases, lyases, isomerases, ligases, oxidoreductases, and translocases.

Steroid sparing agents, such as cyclosporine, non-peptidic hormones such as thyroid hormones, adrenaline, insulin, cortisol, estrogen and progesterone can be used as therapeutic agent cargo.

Anti-microbials such as antibiotics, antivirals, antifungals and antiparasitics can be delivered by the targeting ligands of the present invention. Accordingly, the antimicrobials can be used to prevent and/or treat infections in a subject.

Examples of a chemotherapeutic agent include without limitation: alkylating agents (e.g., which may include doxorubicin, cyclophosphamide, estramustine, carmustine, mitomycin, bleomycin and the like); antimetabolites (e.g., which may include 5-fluoro-uracil, capecitabine, gemcitabine, nelarabine, fludarabine, methotrexate and the like); platinating agents (e.g., which may include cisplatin, oxaliplatin, carboplatin and the like); topoisomerase inhibitors (e.g., which may include topotecan, irinotecan, etoposide and the like); tubulin agents (e.g., which may include paclitaxel, docetaxel, vinorelbine, vinblastine, vincristine, other taxanes, epothilones, and the like); signaling inhibitors (e.g., kinase inhibitors, antibodies, farnesyltransferase inhibitors, and the like); and other chemotherapeutic agents (e.g., tamoxifen, anti-mitotic agents such as polo-like kinase inhibitors or aurora kinase inhibitors, and the like).

Antibodies can be utilized in the methods of the invention. An antibody, or antigen binding fragment thereof, including polyclonal and monoclonal antibodies can be utilized as cargo. The term “antibody fragment” refers to a portion of an immunoglobulin, often the hypervariable region and portions of the surrounding heavy and light chains that displays specific binding affinity for a particular target, typically a molecule. A hypervariable region is a portion of an immunoglobulin that physically binds to the polypeptide target. An antibody fragment thus includes or consists of one or more portions of a full-length immunoglobulin retaining the targeting specificity of the immunoglobulin. Such antibody fragment may for instance lack at least partially the constant region (Fc region) of the full-length immunoglobulin. In some embodiments, an antibody fragment is produced by digestion of the full-length immunoglobulin. An antibody fragment may also be a synthetic or recombinant construct that contains one or more parts of the immunoglobulin or immunoglobulin chains (see e.g., Holliger & Hudson, Engineered antibody fragments and the rise of single domains. Nature Biotechnology 2005, 23(9):1126-1136). Examples of an antibody fragment include, but are not limited to, an scFv, a Fab, a Fv, a Fab′, a F(ab′)2 fragment, a dAb, a VHH, a nanobody, a V(NAR) or a so-called minimal recognition unit.

Another aspect of the invention is a method of treating or preventing a disease in a subject in need thereof, the method comprising administering to the subject a therapeutically effective amount of one or more targeting ligands comprising a cargo effective to treat or prevent the disease, thereby treating or preventing the disease.

In some embodiments, the methods treat or prevent a disease, disorder, or condition by introducing a cargo, thereby modulating the expression of a gene. An example application includes cancer treatments, and gene editing. The administration of a targeting ligand comprising a cargo may increase or enhance expression or reduce or silence gene expression of, for example, a cancer-associated gene.

In another aspect, the methods may be used to detect, diagnose, or monitor a disease, disorder, or condition. Methods of detection can be in vitro and can comprise obtaining a sample from the subject. Suitable samples may comprise, for example, blood, tissue, and solid tumor samples, and enriched forms thereof. According to one embodiment, the sample is a biological sample comprising cells from a subject in need thereof. The biological sample can be from a subject that requires diagnosis of disease, and/or monitoring of the effectiveness of a treatment.

Methods of detection can be in vivo and can comprise administering a targeting ligand comprising an imaging agent or detectable label to the subject. The targeting ligand may comprise a labeled biomarker, which includes nucleic acids, proteins, metabolites and reaction products thereof. Such biomarkers also encompass the mutations, variants, modifications, fragments, and polymorphisms of said biomarkers. Preferably, the subject requires diagnosis of, or is suspected of having, a disease or disorder, and optionally monitoring of the effectiveness of a treatment or progression of a disease. Accordingly, the targeting ligands comprising a cargo such as label or imaging agent are useful in methods of diagnosing, prognosing and/or staging a disease or disorder in a subject by detecting a first level of expression, and/or activity of one or more labels or imaging agents and comparing the detected level to a control of level wherein a difference in the detected level and the control level indicates progression, presence and/or staging of a disease or disorder, for example, cancer.

In an embodiment, methods of targeting a cargo to a cell that comprises one or more B7-H3 receptors are provided, comprising the step of contacting the cell with the B7-H3 targeting ligand. The cell may be a cancer cell comprising one or more B7-H3 receptors. In an aspect, the cancer cell is a lung cell or epithelial cell. The cell may be part of a solid tumor. Methods of targeting a cargo to a cell may be performed in vitro or in vivo. In an aspect, in vitro methods may provide a platform for drug discovery for therapeutically or diagnostically effective cargo molecules, for example, cargo comprising a detectable label, biologically active agent, imaging agent, and/or therapeutic agent.

An aspect of the present invention is a method of delivering a cargo to a cell in vitro. The targeting ligand comprising a cargo may be introduced, e.g., by contact with the cells at the appropriate cargo dosage suitable for the particular target cells. The amount of targeting ligand to administer can vary, depending upon the target cell type and number, and the particular targeting ligand and cargo, and can be determined by those of skill in the art without undue experimentation.

A further aspect of the invention is a method of treating subjects in vivo, comprising administering to a subject one or more targeting ligands each comprising one or more cargo, which may be further comprised in a pharmaceutically acceptable carrier, wherein the pharmaceutical composition is administered in a therapeutically effective amount. Administration of the targeting ligands of the present invention to a human subject or an animal in need thereof can be by any means known in the art for administering compounds. Delivery may be to a lung or epithelial cell. In an aspect, the delivery is to a subject that has, or is suspected of having, cancer.

Example methods can include methods of detecting the presence of cancer in a subject or sample. The targeting ligand can comprise a detectable label or imaging agent, and methods comprise contacting a cell in a sample or subject with the targeting ligand and detecting the presence or level of the imaging agent or detectable label in the cells. Imaging can be by modalities corresponding to the imaging agent or detectable label, e.g., colorimetric output, fluorescence, sequencing or other detection means.

Non-limiting examples of formulations of the invention include those suitable for intravenous administration of the targeting ligands. Oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor) are also envisioned. The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, e.g., a solid tumor, by introduction intravenously at a site near a desired treatment site (e.g., into a vessel that feeds a treatment site, or organ-specific administration with organ specific promoters). In some embodiments, the formulation can be delivered locally to ischemic tissue.

For injection, the carrier will typically be a liquid, such as sterile pyrogen-free water, pyrogen-free phosphate-buffered saline solution, bacteriostatic water, or CREMOPHOR EL® (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

A further aspect of the invention relates to kits for use in the methods of the invention. Kits may comprise one or more B7-H3 targeting ligands; and/or instructions for use, in any combination. The kit can further comprise cargo, carriers, buffers, containers, devices for administration of the components, and the like. The kit can further comprise labels and/or instructions for assay selection and execution. Such labeling and/or instructions can include, for example, information concerning the amount, and methods of administration, detection and quantification for assays detailed herein.

Having described the present invention, the same will be explained in greater detail in the following examples, which are included herein for illustration purposes only, and which are not intended to be limiting to the invention.

### EXAMPLES

### Example 1. MTG Display Selection Against B7-H3 Immune Checkpoint Protein

Microbial transglutaminase (mTG) is a versatile peptide modifying enzyme with intrinsic macrocyclase activity. mTG is formally a cysteine protease that works on the side-chain carboxamides of glutamine residues, cleaving the carboxamide to an enzyme-linked thioester, which is then condensed with available amine nucleophiles, typically the sigma-amino groups of lysine side chains (Gundersen, et al., Appl. Microbiol. Biotechnol. 2014, 98:219-230; Kashiwagi, et al., J. Biol. Chem. 2002, 277:44252-44260). The net reaction effectively allows crosslinking between two peptide-based functional groups in glutamine and lysine (FIG. 1A). This activity has made mTG a versatile tool for a number of commercial applications, including the functionalization of biomacromolecules and as a potent crosslinking biocatalyst (Strop, Bioconjug. Chem. 2014, 25:855-862; Spycher, et al., Chembiochem 2017, 18:1923-1927; Takahara, et al., Bioconjug. Chem. 2017, 28:2954-2961). mTG also has cyclase abilities and can carry out the intramolecular reaction between glutamine and lysine residues to yield isopeptide-bridged macrocycles (Touati, et al., Chembiochem 2011, 12:38-42; Bowen, et al., Int. J. Mol. Sci. 2021, 22 DOI: 10.3390/ijms22041634). This isopeptide linkage is present in a number of natural products, such as microviridins, and has been used to stabilize α-helixes in medicinal chemistry applications, but is not currently accessible in mRNA display (Guarracino, et al., Chem. Rev. 2019, 119:9915-9949; Li, et al., Nat. Chem. Biol. 2016, 12:973-979). Early studies have shown that mTG is compatible with several display platforms and has a good specificity-to-promiscuity profile (Bowen, et al., Int. J. Mol. Sci. 2021, 22 DOI: 10.3390/ijms22041634; Sugimura, et al., Arch. Biochem. Biophys. 2008, 477:379-383; Lee, et al., Biotechnol. Bioeng. 2013, 110:353-362). Despite this foundational work, mTG has not yet been used to cyclize mRNA display libraries for in vitro selection against protein targets of interest.

Efforts to integrate the peptide cyclization activity of mTG with mRNA display are detailed in this example. Applicants developed two distinct high throughput assays to separately assess the substrate tolerance and single round bias of mTG in (1) glutamine activation and (2) lysine cyclization (FIGS. 1B-1C). A high diversity library (~5e¹¹ molecules) was then modified to carry out an affinity selection against a novel cancer target: the immune checkpoint protein, B7-H3. Reference is made to Applicant's publication entitled Enzymatic Macrolactamization of mRNA Display Libraries for Inhibitor Selection, ACS Chem. Biol., 2023, 18(1):166-175, incorporated herein by reference in its entirety.

mTG compatibility and substrate bias during glutamine activation. Efforts were initiated by validating the compatibility of mTG with mRNA displayed substrates. To this end, a capture assay was adopted that was previously deployed in two separate reports by Lee et al. (Biotechnol. Bioeng. 2013, 110:353-362) and Sugimura et al. (Arch. Biochem. Biophys. 2008, 477:379-383). This assay uses pentylamine biotin (PAB) to cleave and capture the mTG-linked intermediate thioester of activated glutamine side chains (FIG. 2A). In this way, sequences that are able to be activated by mTG can be functionalized with PAB and then isolated on streptavidin (SA) resin. In the previous work, the PAB assay had been used over multiple rounds of selection to identify preferred mTG substrates from phage libraries. It was anticipated that a similar version of this assay could be paired with quantitative PCR (qPCR) to test the efficiency of mTG modification of mRNA-linked substrates and also with next-generation sequencing (NGS) to provide coarse grain assessment of substrate promiscuity and substrate bias in a single round of selection.

To this end, a focused display library was prepared, in which three residues on either side of a constant glutamine were comprehensively randomized (FIG. 2B). This library was treated with mTG and submitted to PAB-capture. qPCR revealed that samples treated with mTG recovered better than control, nontreated samples, suggesting that mTG is effective in modifying mRNA linked substrates (FIG. 2C). Both the initial, naïve library and isolated material from the single round capture were submitted for NGS (FIG. 2D). Codon percentage was analyzed in selected material relative to the naïve library (FIGS. 2E-2F); values>1 indicate enrichment in that amino acid and therefore, likely favorable interactions, while values<1 suggest less activation by mTG. At least two composition trends were apparent: (1) aromatic residues were enriched on both sides of the constant glutamine and (2) amino acids that influence flexibility, such as Gly and Pro, were under-enriched at all positions. The deprecation of proline contrasts with the mTG Gln-activation substrates selected by Lee et al. (Biotechnol. Bioeng. 2013, 110:353-362). This can be readily attributed to the decreased pressure and therefore greater number of returned sequences in the single round assay, as compared to multi-round selections in the Lee report. Additionally, although proline is under-enriched in general, several of the Pro-containing substrates from the Lee study were also present in the single round dataset.

With an eye toward use of mTG for cyclization, Applicant also took a close look for the emergence of a ‘WAL’ or similar epitope in the three N-terminal residues. Sugimura and colleagues identified this motif as a directing group for mTG glutamine activation and it has been used in a number of subsequent applications (Sugimura, et al., Arch. Biochem. Biophys. 2008, 477:379-383). Mining the ligation dataset for this epitope showed an abundance of less <0.01% or only 3 out of >30,000 unique sequences. This analysis was further expanded to look for any analogous sequence comprised of an aromatic residue followed by two aliphatics in this position. This analysis still showed a low enrichment over the single round at 0.5% of the isolated library. These data suggest that while WAL may be a preferred substrate motif, there is little bias toward it early in a selection and other residues could likely be enriched by a target if the substrate were randomized at these positions. Overall, no single sequence or sequence family appears enriched (<0.03% sequence convergence) in this initial round, suggesting low substrate bias during glutamine activation.

mTG compatibility and substrate bias during lysine cyclization. Next, modification of the capture assay was sought to examine potential substrate bias during mTG catalyzed macrocyclizations onto lysine residues (FIG. 3A). The macrocyclase activity of mTG is well established and was readily recapitulated in in vitro translation with a model substrate (FIG. 3B). To test this activity in a display context, libraries of Gln-Lys-containing peptide substrates were prepared, all of which were individually fitted with N-terminal biotin tags by Flexizyme-based codon reprogramming. These substrates could be sequentially treated with mTG to effect cyclization and then trypsin protease to discriminate cyclized and linear substrates. Trypsin should cleave at the unmodified lysine in substrates that are not cyclized; trypsin cleavage would remove the N-terminal biotin tag and prevent streptavidin recovery of the displayed substrates in a final step. As with the PAB-Gln activation assay, efficiency could be assessed by qPCR and bias by NGS. Two libraries were prepared for screening in the cyclization assay: one of lower diversity with four randomized positions flanked by Gln and Lys (NNK₄, 1.3e⁵ sequences), and one of medium diversity with six randomized positions flanked by Gln and Lys (NNK₆, 4.7e⁷ sequences) (FIG. 3C). Although the Gln activation data suggested that an N-terminal ‘WAL’ was not necessary, this epitope was included at the N-termini of both cyclization libraries for its potential to anchor the Gln residue (Touati, et al., Chembiochem 2011, 12:38-42; Sugimura, et al., Arch. Biochem. Biophys. 2008, 477:379-383). Under optimized conditions of trypsin loading (FIG. 3D) and incubation time (FIG. 3E), strong recovery (~50%) was observed for mTG treated versus untreated libraries in this assay (FIG. 3F), suggesting that mTG has little difficulty accepting the RNA-tagged peptide substrates.

NGS analysis showed similar conserved trends between the two libraries, NNK₄ and NNK₆ with some notable similarities and differences to the Gln-activation data. Compositional analysis for the recovered sequences revealed high overall sequence diversity for both datasets at <0.06% and <0.01% sequence enrichment for the NNK₄ and NNK₆, respectively. This aligns well with the ligation data and further indicates a lack of single round bias for mTG. Generally, amino acid-level analysis showed a similar overall low degree of variance at all positions for both libraries (FIG. 3G-3K). Similar to the Gln-activation assay, proline was the most under-enriched amino acid, although to a lesser degree here. Aromatic residues were not as highly enriched as in the Gln-activation dataset. Glycine appeared particularly well enriched at position X₂. Differences in reaction type (ligation vs cyclization) and the presence of the WAL motif may contribute to these differences. One other notable trend in the cyclization data was the slight enrichment of Gln residues closer to the C-terminal Lys: positions X₁, X₂, and X₃ in the NNK₄ library and X₃, X₄, and X₅ in the NNK₆ library. Conversely, internal Lys residues seemed somewhat under-enriched (FIG. 3H-3K). These effects, though minor, may reflect a move toward smaller macrocycles. Despite these potentially smaller lactam bridges, a global analysis of the selected sequences showed no proclivity toward secondary structure, especially alpha-helicity (see Methods). Overall, both the activation and cyclization data combined to show that mTG works well on the displayed libraries and suggests that substrate bias may be sufficiently low to be outcompeted by the selective pressure of target binding. Thus, Applicant proceeded with inhibitor selections.

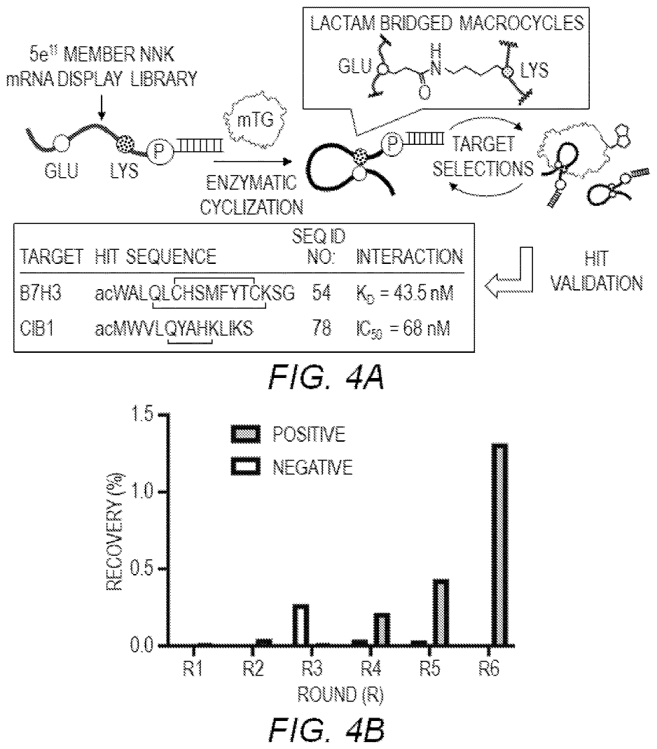

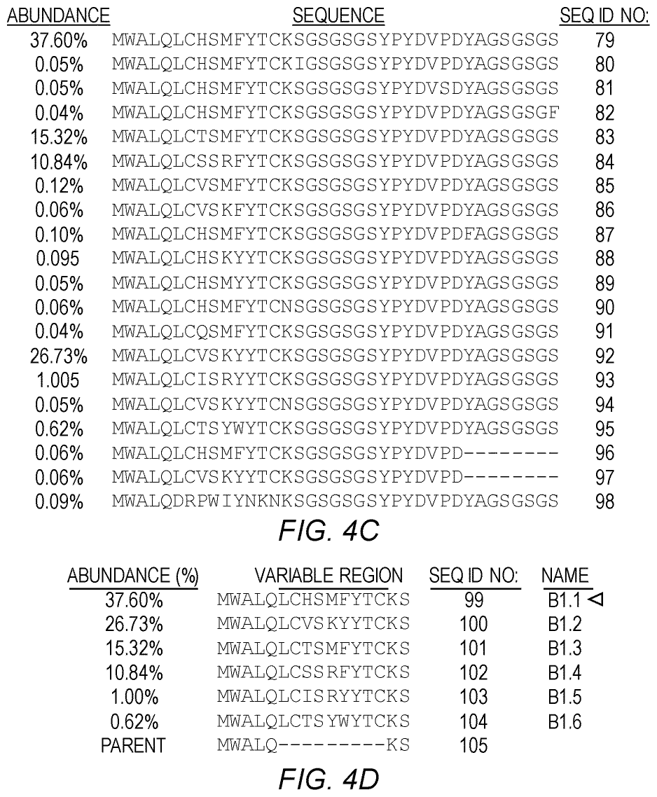

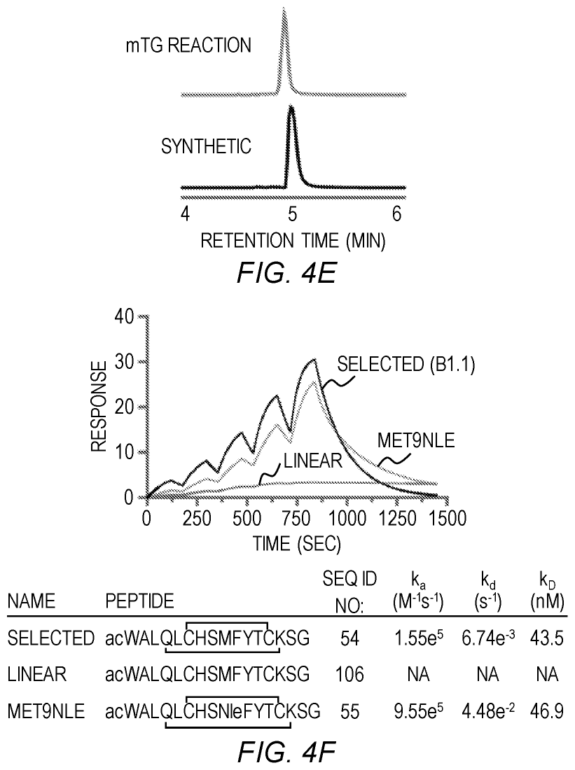

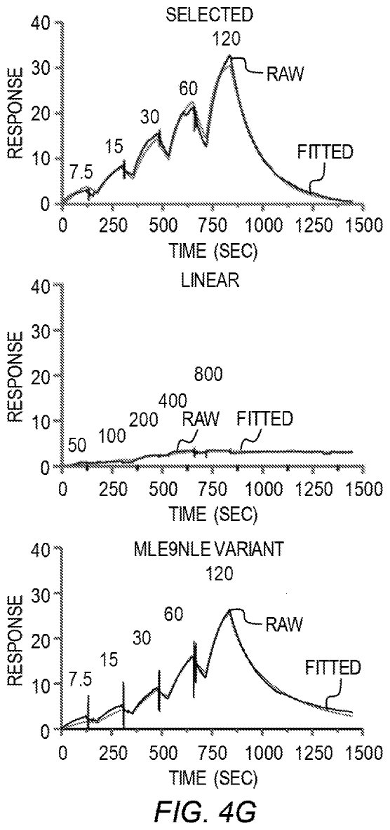

Selection of mTG cyclized B7-H3 Binders. Applicant next sought to use mTG to discover new peptide ligands for a currently unliganded therapeutic target in the immune checkpoint protein B7-H3 (Seaman, et al., Cancer Cell 2017, 31:501-515.e8; Du, et al., Cancer Cell 2019, 35:221-237.e8). Multiple clinical trials are currently underway for biologic-based therapies, such as chimeric antigen receptor T (CAR T) cell and monoclonal antibody-based agents, against B7-H3-expressing cancers (Zhou & Jin, Front. Immunol. 2021, 12:701006). However, no small molecule or low molecular weight scaffolds have been identified as binders for this target. To identify mTG-cyclized peptides against B7-H3, combined NNK₆ and NNK₉ libraries were screened over six rounds against biotin immobilized B7-H3 (FIG. 4A). Enrichment was observed in round 6 at 1.3% of the input (FIG. 4B). NGS revealed strong enrichment of a single B7-H3 family (B1), all members of which came from the NNK₉ library. Sequences in this family displayed cysteines at positions 7 and 14 relative to the N-terminal methionine (FIG. 4C), suggesting the potential selection of a bicyclic disulfide-bridged macrocycle. The top hit (B1.1; FIG. 4D) was included in both linear and bicyclic (both lactam and disulfide) forms by solid phase peptide synthesis (SPPS). To simplify synthesis, Applicant opted not to include the N-terminal methionine in the synthetic peptides, as it is prone to oxidation and also prepared the Met9Nle in parallel because of similar concerns. Notably, while there are potentially multiple different interlocking conformations possible with the bicyclic B1.1, the synthetic material had an identical retention time via LCMS as enzymatically cyclized material prepared under the display conditions (FIG. 4E). Binding affinities were assessed for each peptide by surface plasmon resonance (SPR) against biotinylated B7-H3. Notably, B1.1 displayed a 43.5 nM binding affinity towards B7-H3 and the Met9Nle variant was similar (FIG. 4F). Meanwhile, the linear variant of B1.1 showed no detectable binding interaction (FIG. 4F). This data demonstrates necessity for cyclization of B1.1 for binding and strongly suggest its influence in the identification of these cyclized B7-H3 hits. Overall, these results show that this method is capable of identifying potent, nanomolar cyclic binders of new targets.

Discussion. This work shows the potential power of merging enzymatic post-translational modifications with mRNA display. Applicant developed display-based assays that show the compatibility of mTG activation and cyclization with mRNA display libraries and begin to assess the substrate bias of the enzyme. mTG adds a novel, structurally distinctive, and pharmaceutically relevant isopeptide bond to the small but growing list of macrocyclization chemistries that are now accessible to mRNA display libraries. Notably, this chemistry is distinguished by its orthogonality to substrate cysteine-residues, which are commonly employed as cyclization handles; mTG cyclization leaves substrate cysteines open for alternative modification chemistries that can be used to broaden library diversity.

B7-H3 binding peptide, B1.1 and its Met9Nle variant, provide novel scaffolds for development as diagnostic probes or therapeutic delivery systems to B7-H3 expressing cancers. Both the substrate display results and the target selections provide insights into the opportunities and challenges of enzyme-modified display campaigns.

The substrate display data suggests that mTG is broadly promiscuous, although there is a level of substrate bias that may be able to work its way into selections. It is important to note the limitations of this current promiscuity assessment: due to sequencing constraints, Applicant did not cover the full theoretical diversity of several of the libraries in these experiments. Future experiments, employing more extensive, deep sequencing analysis or other methods can comprehensively analyze the substrate bias of this and similar candidate enzymes. Still, most of the reads recovered for the substrate display experiments are for unique sequences and some changes in position level composition are statistically significant, contributing to several identifiable trends. One significant finding from the activation (PAB) assay is that a recognition element (e.g., the WAL epitope) directly around the Gln residue undergoing activation may not be required and Gln residues in diverse peptide contexts may be able to undergo activation by mTG. Similarly, in the cyclization assays there may be a bias towards new cyclization partner residues (Gln and Lys) in closer proximity to the fixed residues. The latter result suggests that partner spacing and thus ring size might have a stronger impact on cyclization pairs than sequence context. Substrate conformation and/or the general electrostatic character of the enzyme active site may play roles in selecting which ring sizes are preferred. A deep sequencing analysis of these assays could further elucidate these potential trends and begin to distinguish kinetic versus thermodynamic control in the biocatalytic context. Designer DNA sequences and libraries could be used to control these substrate biases especially in placement of the Gln and Lys cyclization partners. Substrate bias did not prevent selection of diverse peptide macrocycles in pans against B7-H3.

Conclusion. Together, these steps, substrate display followed by ligand selection, suggest a model framework for introducing an enzyme into the mRNA display toolkit and pinpoint several of the knowledge gaps to be traversed in the process. Enzymatic post-translational modifications present a rich and appealing toolset for mRNA display diversification (Hudson & Mitchell, Curr. Opin. Microbiol. 2018, 45:61-69; Montalbán-López, et al., Nat. Prod. Rep. 2021, 38:130-239). Although further studies may provide insights into preferred cyclization methods for a given target class, it is anticipated that access to more cyclization methods and thus greater structurally diversity can only benefit naïve panning campaigns (Hacker, et al., ACS Comb. Sci. 2020, 22:306-310). Perhaps in future applications, multiple enzymatic modifications could be mixed or layered together as in natural product biosynthesis, to enable screening of ever more complex and privileged scaffold libraries. Applicant anticipates that access to such libraries would aid target engagement, serum stability, and cell penetrance of next generation inhibitors and macrocyclic therapeutics.

Materials. Reagents for molecular biology were purchased from New England BioLabs (NEB), Thermo Fisher Scientific, Fisher Scientific, and Zedira (Darmstadt, Germany). Specifically, PURExpress® Kits (E6840S—standard translations, and E6850Z—custom for mRNA display), T7 RNA Polymerase (M0251L), Q5 DNA Polymerase (M0491L) were purchased from New England Biolabs. T4 RNA. ligase I (PR-M1051), RQ1 DNase (PR-M6101), and M-MLV Reverse Transcriptase, RNase H Minus, Point Mutant (PRM3683) were purchased from Promega through Fisher Scientific. Anti-HA magnetic beads (88836), HA synthetic peptide (26184), and M-280 Streptavidin Dynabeads™ (11205D) were purchased from Thermo Fisher Scientific. Bulk solvents were purchased from Fisher Scientific. Most fmoc-amino acids and materials for peptide synthesis were purchased from Chemimpex. Microbial Transglutaminase (T001) was purchased from Zedira. DNA gene fragments were purchased from Twistbioscience. Primers were purchased from Integrated DNA Technologies. Next Generation Sequencing (NGS) was performed by Azenta using their Amplicon-EZ (150-500 bp) service.

Instrumentation. Preparatory high pressure liquid chromatography (HPLC) was carried out on a Shimadzu UFLC CBM-20A with a dual channel wavelength detector at 220 and 254 nm and over a Phenomenex LUNA 10 μm C18 100 A, AXIA semi-preparatory column at a flow rate of 15 mL/min. For purification a two solvent system (Solvent A: 0.1% Trifluoracetic acid and water; Solvent B: 0.1% Trifluoracetic acid and acetonitrile) was used on a gradient from A to B. For analysis of peptide synthesis and the mTG reaction, an Agilent 6520 Accurate-Mass Q-TOF High resolution Liquid Chromatography and Mass Spectrometry (LCMS) with a Kinetex 2.6 u C18 column was employed. MALDI-TOF analysis was carried out on an Applied Biosystems SIEX TOF/TOF 5800 system in reflector positive mode. An Applied Biosystems (AB) ViiA 7 Real-Time PCR System (qPCR) was used for amplification and analysis after mRNA display experiments.

Software. Software used for data analysis comprised of Agilent Masshunter Qualitative Analysis Navigator B.08.00 (LCMS analysis), Qiagen CLC Main Workbench (NGS analysis), Anaconda navigator Jupyter Notebook v6.3.0 (Python Script), Graphpad Prism v9.2.0, and Microsoft office suite. Adobe Illustrator was used to assemble figures. Finally, in house python scripts were used to analyze NGS data.

Reconstitution of microbial transglutaminase (m/C). Lyophilized mTG (T001) was purchased from Zedira and reconstituted following manufacture's protocol. In brief, the lyophilized powder was resuspended in Milli-Q H₂O (MQ-H₂O) based on the reported lyophilization volume (solution lyophilized from was 50 mM NaOAc pH 5.0, 300 mM NaCl). Then an equal volume of 100 mM Tris-Cl pH 8.0, 10% glycerol was added to yield a 1× working stock of mTG at 50 mM in 50 mM Tris-Cl pH8.0, 25 mM NaOAc, 150 mM NaCl, 5% glycerol. This was aliquoted into low use portions and stored at −80° C.

Complete Peptide, Gene, and Library Sequences. Control Fusion for MALDI Analysis (Purchased as Single Gene Fragment).

Peptide sequence:
(SEQ ID NO: 26)
MWALQRPHGGGKSG
DNA sequence:
(SEQ ID NO: 27)
CACTTCGGGCTCATGAGCGCTTGTTTCGGCGAAATTAATACGACTCACT
ATAGGGGAATTGTGAGCGGATAACAATTCCCCTCTAGAAATAATTTTGT
TTAACTTTAAGAAGGAGATATACATATGTGGGCGTTGCAACGTCCGCAT
GGCGGCGGTAAAAGTTAATAGCGCATTGGAAGTGGATAACGGATCCGAA
TTCGAGCTCCGTCGACAAGCTTGCGGCCGCACTCGAGTGAGATCCGGCT
GCTAACAAAGCCCGAAAGGAAGCTGAGTTGGCTGCTGCCACCGCTGAGC
AATAACTAGCATAACC

Control Fusion for mRNA Display Analysis (Purchased as Single Gene Fragment)

Peptide sequence:
(SEQ ID NO: 28)
MWALQRPHGGGKSGSGSGSYPYDVPDYAGSGSGS
DNA sequence:
(SEQ ID NO: 29)
TAATACGACTCACTATAGGGTTAACTTTAAGAAGGAGATATACATATGT
GGGCTTTACAGCGTCCACACGGAGGAGGAAAATCGGGTTCAGGTAGCGG
AAGTTACCCGTACGATGTTCCTGACTATGCCGGCAGTGGGTCGGGATCC
TAGGACGGGGGGCGGAAA

Lib1. mTG-NNK₆ Ligation Library (Assembled from Primers).

Peptide sequence:
(SEQ ID NO: 30)
MXXXQXXXGSGSGSYPYDVPDYAGSGSGS, X = NNK or 20/20
amino acids and only the TAG stop codon
DNA sequence:
(SEQ ID NO: 31)
TAATACGACTCACTATAGGGTTAACTTTAAGAAGGAGATATAAATATGN
NKNNKNNKCAANNKNNKNNKAGCGGGAGTGGCAGCGGCAGCTACCCATA
CGACGTGCCCGACTATGCAGGTTCTGGTTCTGGTTCTTAGGACGGGGGG
CGGAAA

Lib2. mTG-NNK₄ Cyclization Library (Assembled from Primers).

Peptide sequence:
(SEQ ID NO: 32)
MWALQ(X)₄KSGSGSGSYPYDVPDYAGSGSGS, X = NNK or
20/20 amino acids and only the TAG stop codon
DNA sequence:
(SEQ ID NO: 33)
TAATACGACTCACTATAGGGTTAACTTTAAGAAGGAGATATAAATATGT
GGGCTTTACAANNKNNKNNKNNKAAGAGCGGGAGTGGCAGCGGCAGCTA
CCCATACGACGTGCCCGACTATGCAGGTTCTGGTTCTGGTTCTTAGGAC
GGGGGGCGGAAA

### NO:33)

Lib3. MTG-NNK₆ Cyclization Library (Assembled from Primers).

Peptide sequence:
(SEQ ID NO: 34)
MWLQ(X)₆KSGSGSGSYPYDVPDYAGSGSGS, X = NNK or 20/20
amino acids and only the TAG stop codon
DNA sequence:
(SEQ ID NO: 35)
TAAACGACTCACTATAGGGTTAACTTTAAGAAGGAGATATAAATATGTG
GGCTTTACAANNKNNKNNKNNKNNKNNKAAGAGCGGGAGTGGCAGCGGC
AGCTACCCATACGACGTGCCCGACTATGCAGGTTCTGGTTCTGGTTCTT
AGGACGGGGGGCGGAAA

Lib4. mTG-NNK₉ Cyclization Library (Assembled from Primers).

Peptide sequence:
(SEQ ID NO: 36)
MWALQ(X)₉KSGSGSGSYPYDVPDYAGSGSGS, X = NNK or
20/20 amino acids and only the TAG stop codon
DNA sequence:
(SEQ ID NO: 37)
TAATACGACTCACTATAGGGTTAACTTTAAGAAGGAGATATAAATATGT
GGGCTTTACAANNKNNKNNKNNKNNKNNKNNKNNKNNKAAGAGCGGGAG
TGGCAGCGGCAGCTACCCATACGACGTGCCCGACTATGCAGGTTCTGGT
TCTGGTTCTTAGGACGGGGGGCGGAAA

TABLE 1
Primers List

| # | Primer Name | Sequence 5′-3′ |
|---|---|---|
| P1 | T7 Prom.F.26 | GAAATTAATACGACTCACTATAGGGG (SEQ ID NO: 38) |
| P2 | T7 Term.R.19 | GCTAGTTATTGCTCAGCGG (SEQ ID NO: 39) |
| P3 | T7 NNK F.46 | TAATACGACTCACTATAGGGTTAACTTTAAGAAGGAGATAT |
| | | AAATA (SEQ ID NO: 40) |
| P4 | Plinking Prep | TTTCCGCCCCCCGTCCTAAGAACCAGAACCAGAACCTGCA |
| | R.75 | TAGTCGGGCACGTCGTATGGGTAGCTGCCGCTGCC (SEQ |
| | | ID NO: 41) |
| P5 | mTG-NNK₄ R.91 | GTCGTATGGGTAGCTGCCGCTGCCACTCCCGCTCTTMNNM |
| | cyclization | NNMNNMNNTTGTAAAGCCCACATATTTATATCTCCTTCTTA |
| | | AAGTTAACCC (SEQ ID NO: 42) |
| P6 | mTG-NNK₆ R.97 | GTCGTATGGGTAGCTGCCGCTGCCACTCCCGCTCTTMNNM |
| | cyclization | NNMNNMNNMNNMNNTTGTAAAGCCCACATATTTATATCTC |
| | | CTTCTTAAAGTTAACCC (SEQ ID NO: 43) |
| P7 | mTG-NNK₉ | GTCGTATGGGTAGCTGCCGCTGCCACTCCCGCTCTTMNNM |
| | R.106 | NNMNNMNNMNNMNNMNNMNNMNNTTGTAAAGCCCACA |
| | cyclization | TATTTATATCTCCTTCTTAAAGTTAACCC (SEQ ID NO: 44) |
| P8 | mTG-NNK₆ R.85 | GTCGTATGGGTAGCTGCCGCTGCCACTCCCGCTMNNMNNM |
| | ligation | NNTTGMNNMNNMNNCATATTTATATCTCCTTCTTAAAGTTA |
| | | ACCC (SEQ ID NO: 45) |
| P9 | NNK Plinker | TTTCCGCCCCCCGTCCTAAGAACCAGAACCAGAACCTGC |
| | R.39 | (SEQ ID NO: 46) |
| P10 | NNK qPCR F.28 | GAAGGAGATATAAATATGTGGGCTTTAC (SEQ ID NO: 47) |
| P11 | NNK qPCR R.22 | CCTAAGAACCAGAACCAGAACC (SEQ ID NO: 48) |
| P12 | NGS NNK | ACACTCTTTCCCTACACGACGCTCTTCCGATCTTAATACGA |
| | adaptor F.58 | CTCACTATAGGGTTAAC (SEQ ID NO: 49) |
| P13 | NGS NNK | GACTGGAGTTCAGACGTGTGCTCTTCCGATCTTTTCCGCCC |
| | adaptor R.58 | CCCGTCCTAAGAACCAG (SEQ ID NO: 50) |
| P14 | Clonal R.22 | TTTCCGCCCCCCGTCCTAGCTG (SEQ ID NO: 51) |

Gene fragment PCR. Gene fragments were first resuspended to a final concentration of 10 ng/μL. Then a 100 μL PCR amplification was carried out under standard Q5 DNA polymerase protocols—briefly, the reaction contained 1× Q5 Reaction buffer, 2.5 mM MgCl₂, 0.25 mM dNTPs, 0.5 μM forward primer (P1 for MALDI substrates or P3 for display substrates), 0.5 μM reverse primer (P2 for MALDI substrates or P9 for display substrates), 0.2 ng/μL DNA template, 0.02 units/μL Q5 DNA Polymerase (NEB M0491L), and diluted in MQ-H₂O. This was carried out under conditions listed below in the PCR amplification table. Amplification was confirmed by 3% agarose gel (supplemented with 1.5% ethidium bromide). The DNA was then purified by a PCR purification kit and isolated in MQ-H₂O. The concentration was determined by nanodrop and then stored at −20° C. until further use.

TABLE 2
PCR-1 amplification conditions:

| Step | Temperature (° C.) | Time (sec) | |
|---|---|---|---|
| Initial Denature | 95 | 60 | |
| Denature | 95 | 40 | 30 cycles |
| Annealing | 63 | 40 | |
| Elongation | 72 | 40 | |
| Final Elongation | 72 | 120 | |

RNA preparation. Single gene substrates for mRNA display analysis were then transcribed using an adapted T7 RNA Polymerase protocol (NEB, M0251L). A 20 μL reaction was setup and contained 1× T7 RNA Polymerase Buffer, 1 mM Dithiothreitol (DTT), 16.5 mM MgCl₂, 5 mM rNTPs, 25 ng/μL DNA template, and 5 units/μL T7 RNA Polymerase. This was incubated overnight at 37° C. Successful transcription reactions appear cloudy due to the precipitation of magnesium pyrophosphate. If precipitation was observed then the RNA was extracted from the solution. To carry out the extraction 1× volume of MQ-H₂O (20 μL), and 2× volume of crashout solution (0.6M NaCl and 50 mM EDTA) was added. To this mixture, 1× volume of phenol/chloroform/isoamyl alcohol was added and centrifuged at 16,000×g. The top layer was extracted, transferred to a clean tube, and 1× volume of chloroform/isoamyl alcohol was added and centrifuged. The top layer was again extracted, transferred to a clean tube, and then 0.64× volume of isopropyl alcohol was added and the solution centrifuged at 16,000×g for 10 mins to pellet the RNA. The supernatant was then removed and excess 70% ethanol was added, centrifuged briefly at 16,000×g, and then the RNA pellet was dried at room temperature (RT). This was solubilized in MQ-H₂O and the concentration was determined by nanodrop. This solution was stored at −20° C. until use.

DNA preparation. An initial extension reaction (100 μL) containing 1× Q5 Reaction buffer, 2.5 mM MgCl₂, 0.25 mM dNTPs, 0.5 μM forward primer (P3), 0.5 μM reverse primer (P5, 6, 7, or 8—specific for each library), 0.02 units/μL Q5 DNA Polymerase (NEB M0491L), and diluted in MQ-H₂O. This was run for 5 cycles on a thermocycler at conditions listed in the extension reaction table (below). Once complete a 1 mL PCR amplification was set up containing all of the initial extension reaction, 1× Q5 Reaction buffer, 2.5 mM MgCl₂, 0.25 mM dNTPs, 0.5 μM forward primer (P3), 0.5 μM reverse primer (P4), 0.02 units/μL Q5 DNA Polymerase (NEB M0491L), and diluted in MQ-H₂O. This was carried out under conditions listed below in the PCR-2 amplification table. The progress of this reaction was checked by 3% agarose gel (supplemented with 1.5% ethidium bromide). Once sufficient amplification 1× volume of phenol/chloroform/isoamyl alcohol was added to the solution and centrifuged. The top layer was extracted, transferred to a clean tube, and 1× volume of chloroform/isoamyl alcohol was added to the solution and centrifuged. The top layer was again extracted, transferred to a clean tube, and then a 1/10 volume of 3M NaCl and 2× volume of 100% ethanol was added. This mixture was centrifuged for 10 mins to pellet the DNA. Once pelleted, the DNA was washed with excess 70% ethanol, then allowed to dry at RT. The DNA was then solubilized in a 1/10 volume (100 μL) of the PCR reaction.

TABLE 3
Extension reaction conditions:

| Step | Temperature (° C.) | Time (sec) | |
|---|---|---|---|
| Initial Denature | 95 | 60 | |
| Annealing | 68 | 60 | 5 cycles |
| Elongation | 72 | 60 | |
| Final Elongation | 72 | 120 | |

TABLE 4
PCR-2 amplification conditions:

| Step | Temperature (° C.) | Time (sec) | |
|---|---|---|---|
| Initial Denature | 95 | 60 | |
| Denature | 95 | 40 | 10 cycles |
| Annealing | 68 | 40 | |
| Elongation | 72 | 40 | |
| Final Elongation | 72 | 120 | |

RNA transcription and Urea gel extraction. A 1 mL transcription reaction (adapted from NEB T7 RNA polymerase protocols) was set up and contained all of the amplified DNA, 1× T7 RNA Polymerase Buffer, 1 mM Dithiothreitol (DTT), 16.5 mM MgCl₂, 5 mM rNTPs, and 5 units/μL T7 RNA Polymerase (M0251L). This solution was incubated overnight at 37° C. Upon successful transcription magnesium pyrophosphate precipitates and the solution appears cloudy. To this solution 100 μL of 10× DNase I buffer and 30 μL of DNase I was added and the solution incubated at 37° C. for 1 h. To this solution EDTA, NaCl, and 100% Isopropyl alcohol was added to final concentrations of 37.5 mM, 150 mM, 45%, respectively. Further, the solution was incubated on ice, after EDTA addition, until the solution became clear. If this did not occur then additional EDTA was added. The final solution was then centrifuged 16,000×g for 10 mins to pellet the RNA. Then the supernatant was removed and excess 70% ethanol was added. This solution was briefly centrifuged at 16,000×g, the supernatant removed, and the RNA pellet was dried at RT. Next the RNA was solubilized in 100 μL of MQ-H₂O and an equal volume (100 μL) of 2×RNA loading dye was added. This was heated at 95° C. for 2 mins and then purified on a large scale 8% Urea-Page gel. This gel was run at 230V for 1.5 h in a Tris-Borate-EDTA (TBE) buffer. The RNA was then visualized under UV illumination at 254 nm on a silica-coated thin-layer chromatography plate and excised from the gel. The excised gel, containing the RNA, was crushed into fine pieces and the RNA extracted with 0.3M NaCl (2×1 h incubation at RT or 1× overnight at 4° C.). To collect the RNA, the gel was pelleted by centrifugation at 16000×g and the supernatant isolated. Then the supernatant was passed through a 0.45 μm filter into a clean 50 mL conical tube. To the flow through 2× volume of 100% ethanol was added and the resulting mixture was centrifuged at 16,000×g for 10 mins to crashout the RNA. The pellet was washed with excess 70% ethanol, briefly centrifuged at 16,000×g, and then dried at RT. The RNA was solubilized in MQ-H₂O and the concentration was determined by nanodrop. This material was stored at −20° C. until use.

Linking puromycin to RNA libraries (Plinking). RNA for each library was covalently linked to puromycin via an adapted Y-ligation strategy (Sohrabi, et al., Nat. Rev. Chem. 2020, 4:90-101). The reaction was carried out in MQ-H₂O with 1 μM RNA, 20% DMSO, 1.5 μM P-Linker (d(pCTCCCGCCCCCCGTCC (SEQ ID NO:52))-(SPC18)5-d(CC)-puromycin, wherein Spc18 represents the spacer-phosphoramidite 18), 1× T4 RNA ligase buffer, and 1 unit/μL T4 RNA ligase I (PR-M1051). Once assembled the reaction was incubated at 37° C. for 0.5 h. After incubation 1× volume of crashout solution (0.6M NaCl, and 50 mM EDTA), 0.02× volume of 100% glycogen, and 2× volume 100% ethanol were added. This mixture was briefly vortexed and then centrifuged at 16,000×g for 10 mins to pellet the Plinked RNA. The supernatant was removed and excess 70% ethanol was added, briefly centrifuged at 16,000×g, supernatant removed, and the pellet was dried at RT. Once dried the Plinked RNA pellet was reconstituted in an equal volume as the input RNA in MQ-H₂O. The efficiency of the reaction was determined by 8% Urea Gel (National Diagnostics, EC-833).

In vitro translation (IVT) assays to assess cyclization. All IVT assays were carried out using PURExpress® (aa, tRNA) Kit (E6840S) from NEB on a 5 μL scale. The solution was first incubated at 37° C. for 0.5 h to facilitate translation. Next, the peptide was purified from the IVT solution using a C18 Spin tip (Pierce P184850). To prepare the C18 spin tip, 10 μL of a C18 elution solution (80% acetonitrile and 0.5% acetic acid in water) was added, spun down, and flow through removed. Then 10 μL of a C18 elution solution (4% acetonitrile and 0.5% acetic acid in water) was added, spun down, and flow through removed. Next the translation solution was added, washed twice with the wash solution, then eluted into a clean tube with 1 μL of elution solution. The elution solution was then evaporated off with N₂ and the peptide was resuspended in 10 μL of 50 mM Tris-Cl pH8.0, 200 mM NaCl. This was split into equal portions and mTG was added at a final concentration of 4 μM or left untreated. This mixture was incubated at 37° C. for 1 h. Then another C18 Spin Tip was prepared as above, each sample was added to the tip and washed twice with wash buffer. Finally, 1.5 μL of a half-saturated solution of α-cyano-4-hydroxycinnamic acid in elution buffer was used to elute the peptide from the C18 tip. This was spotted on a MALDI-TOF plate and analysis was analysis was carried out on an AB SIEX TOF/TOF 5800 system in reflector positive mode.

N-Biotin-L-Phenylalanine initiator tRNA acylation. Preparation of N-Biotin-L-Phenylalanine initiator tRNA cyanomethyl ester (Bio-Phe-itRNA CME) was prepared as previously described (Iskandar, et al., ACS Comb. Sci. 2020, 22:712-733). Acylation of initiator tRNA (itRNA) was carried out in a final solution of 83 mM HEPES-KOH (pH 7.5), 42 μM Flexizyme (eFx), 42 μM itRNA, 600 mM MgCl₂, 5 mM of Bio-Phe-itRNA CME, 20% DMSO (from Bio-Phe-itRNA CME), and MQ-H₂O. To carry out, MQ-H₂O, HEPES-KOH (pH 7.5), eFx, itRNA were combined and heated at 95° C. for 2 mins, then the solution was incubated at RT for 5 mins. Next, MgCl₂ was added and incubated at RT for 5 mins. Finally, Bio-Phe-itRNA CME was added and the solution was incubated on ice for 2 h. Once incubation was complete the acylated itRNA was crashed-out with a solution of 0.3M NaAcO and 70% ethanol. The pelleted was air dried and stored (as a pellet) at −80° C. until use.

General translations anti reverse transcription procedure for mRNA display. Translations for mRNA display were carried with a customized NEB PURExpress® kit (-aa, -tRNA, -RF123) -E6850Z. A solution containing 1× SolA (supplied as 5× stock), 0.5 mM tRNAs, 1.2 mM Plinked mRNA, 0.5 mM Amino acid mix, MQ-H₂O (to reach final volume), and 1× SolB (added last to initiate translation, supplied as 3.3× stock) was prepared. The solution was incubated at 37° C. for 30 mins, followed by a 10 min incubation at RT to facilitate fusion of peptide to its mRNA strand. Finally, EDTA. was added to a final concentration of 17 nM, to dissociate the ribosome, and the mixture was incubated at 37° C. for 20 mins. Next, complementary DNA was added by a reverse transcription reaction containing all the translation product, 0.6 mM dNTPs, 5 uM reverse primer (P9), 62.5 mM Tris-HCl pH 8.3, 37.5 mM Mg(OAc)₂, 25 mM KOH, 2.5× M-MLV reverse transcriptase H (−), point mutant (Promega, M3681, supplied at 40×), and MQ-H2O (to reach final volume). This solution was incubated at 42° C. for 1 h. After incubation an analytical sample (0.5 uL of translation/reverse transcription product diluted into 250 mL MQ-H₂O) was taken for qPCR analysis of initial translation.

Ligation analysis Lib1. mTG-NNK₆ ligation library. Plinked mTG-NNK₆ Ligation mRNA (Lib1.) was translated using a full complement amino acid mix (20/20AA). A 10 μL reaction (set up in triplicate) was prepared, translated, and transcribed as stated above. Once complete the fusions were purified using anti-HA magnetic beads at a concentration of 4 mL bead slurry to 1 mL of IVT. This mixture was then diluted 10× from the original translation volume (e.g., 100 mL) and incubated, while rotating, at RT for 0.5 h. Once complete, the beads were washed 3× with 1×TBST (50 mM Tris-HCl pH 8.0, 200 mM NaCl, 0.05% TWEEN® 20). The fusion bound bead solution was then concentrated to 40 μL and then split into two equal portions. Each portion was treated (+mTG) or left untreated (−mTG). To carry out a 100 μL reaction was set up containing 4 μM mTG, 20 μL of fusion-bead sample, 1 mM pentylamine biotin (ThermoFisher Scientific, 21345—prepared in MQ-H₂O as a 10 mM stock solution), and diluted to the final volume with 1×TBST. The untreated sample was diluted to the sample volume as the (+) mTG sample (100 μL) with 1×TBST. Both samples were then incubated on a 37° C. benchtop shaker (to prevent the anti HA beads from clumping) for 1 h. After incubation each solution was washed 3× with 1×TBST. The samples were eluted from the anti-HA beads in a 50 μL solution containing 25 μL of 4 mg/mL HA synthetic peptide (prepared in MQ-H₂O) and 2×TBST. Elution was carried out for 1 h at RT. Once complete magnetized Streptavidin beads (4 mL of streptavidin beads/1 mL original translation reaction in each sample—e.g., 20 μL of SA beads to 5 μL of IVT) were added to each sample to capture any biotinylated material. Samples were incubated while rotating at 4° C. for 0.5 h then washed 3× with 1×TBST. After the final wash each sample was brought up in 1×PCR buffer (10 mM Tris-HCl pH 9.0, 50 mM KCl, and 0.1% TRITON X-100) and incubated at 95° C. for 5 mins to dissociate cDNA from each fusion. The supernatant was collected and the amount of recovered cDNA was determined by qPCR (see below). Recoveries were analyzed as a function of the HA purified input amount. Isolated cDNA was PCR amplified based on the CT value from the qPCR results using standard Q5 polymerase protocols and primers (P3, P9—annealing temperature of 57° C.). Once sufficient amplification was observed then the DNA was crashed out using a 0.1× (v/v) of 3M NaCl and 2× (v/v) of 100% ethanol. This was spun down at 15000 rpm for 10 mins, supernatant removed, washed with 70% EtOH and then allowed to dry.

Trypsin titration selection assays—control sequence. Plinked mTG-Control mRNA was translated with a 19AA amino acid mix (0.5 mM in solution) lacking methionine. The translation was supplemented with 50 mM Bio-Phe-itRNA (prepared as stated above and solubilized in 1 mM NaOAc pH5.2). A 16 mL reaction was prepared, translated, and transcribed as stated above. Once complete the fusions were purified using anti-HA magnetic beads at a concentration of 4 mL bead slurry to 1 mL of IVT. This mixture was then diluted 10× from the original translation volume (e.g., 160 mL) and incubated while rotating at RT for 0.5 h. Once complete, the beads were washed 3× with 1×TBST (50 mM Tris-HCl pH 8.0, 200 mM NaCl, 0.05% TWEEN® 20) and brought up in 0.5× of original binding volume (80 mL). This was then split into two equal portions. To one portion mTG was added to a final concentration of 4 mM (saturating equivalence of mTG to fusion) and then diluted to 80 mL with 1×TBST. The other portion was left untreated and diluted to a final volume of 80 μL. Both samples were incubated on a 37° C. stand mixer (to ensure the beads remain suspended) for 1 h, After incubation, both samples were washed with 1×TBST, to remove mTG from solution, then resuspended with 40 mL (0.5× volume) of 2×TBST and 40 μL of 4 mg/mL HA peptide (to elute the fusion from the anti-HA bead). This was carried out at RT for 1 h. The final elution volume for each portion was 80 mL. Both samples were split out into 10 mL aliquots (~1 mL of original translation volume). Trypsin, prepared at 10 mM stock in 50 mM acetic acid, (MS/MS Pearce, 90057) was added at concentrations ranging from 0 to 800 nM. Samples were rotated at 4° C. for 0.5 h. Once complete magnetized Streptavidin beads (4 mL of streptavidin beads/1 mL original translation reaction) were added to each sample followed by 2× volume of 1×TBST (to dilute trypsin). Samples were incubated while rotating at 4° C. for 0.5 h then washed 3× with 1×TBST. After the final wash each sample was brought up in 1×PCR buffer (10 mM Tris-HCl pH 9.0, 50 mM KCl, and 0.1% TRITON X-100) and incubated at 95° C. for 5 mins to dissociate cDNA from each fusion. The supernatant was collected and the amount of recovered cDNA was determined by qPCR (see below). Recoveries were analyzed as a function of the non-trypsin digested sample (0 nM).

mTG time course assay Lib2. NNK₄ cyclization library. Plinked mTG-NNK₄ mRNA (Lib2.) was translated with a 19AA amino acid mix lacking methionine. The translation was supplemented with 50 mM biotin-Phe-initiator tRNA (solubilized in 1 mM NaOAc pH5 2). A 25 mL reaction was prepared, translated, and transcribed as stated above. Once complete the fusions were purified using anti-HA magnetic beads at a concentration of 4 mL bead slurry to 1 mL of IVT. This mixture was then diluted 10× from the original translation volume (e.g., 250 mL) and incubated while rotating at RT for 0.5 h. Once complete, the beads were washed 3× with 1×TBST (50 mM Tris-HCl pH 8.0, 200 mM NaCl, 0.05% TWEEN® 20) and brought up in 250 mL of 1×TBST. Then 40 mL (~4 mL original IVT) was taken out washed 3× with 1×TBST and stored on ice in 1×TBST—this was the 0 min timepoint sample. The remaining 210 mL was then concentrated and resuspended in 1×TBST supplemented with 4 μM of mTG. This was added to a 37° C. stand mixer and 40 μL samples taken at 3.75, 7.5, 15, 30, and 60 mins. Each sample was washed in a similar fashion to the 0 min timepoint sample. After the time course incubation each aliquot was concentrated and resuspended in % volume (eq 20 μL) 2×TBST and % volume solution of 4 mg/mL HA synthetic peptide (prepared in MQ-H₂O). This was mixed on a rotator at RT for 1 h. Following incubation, the supernatant was collected (contains eluted fusion) and each sample split into equal portions (~2 μL IVT, 20 μL total). To one of each sample a solution of 20 μL of 0.2 nM trypsin (MS/MS Pearce, 90057) was added. The trypsin was prepared as a 10 μM stock in 50 mM acetic acid. To the other sample 20 μL of 1×TBST was added. Both sample types were incubated on a rotator at 4° C. for 0.5 h. Once complete magnetized Streptavidin beads (4 mL of streptavidin beads/1 mL original translation reaction) were added to each sample followed by 2× volume of 1×TBST (to dilute trypsin). Samples were incubated while rotating at 4° C. for 0.5 h then washed 3× with 1×TBST. After the final was each sample was brought up in 1×PCR buffer (10 mM Tris-HCl pH 9.0, 50 mM KCl, and 0.1% TRITON X-100) and incubated at 95° C. for 5 mins to dissociate cDNA from each fusion. The supernatant was collected and stored at 4° C. until ready for qPCR analysis (see qPCR analysis section for sample preparation). Recoveries were analyzed as a function the corresponding non-trypsin digested sample (e.g., 1 h mTG [+trypsin]/1 h mTG [−Trypsin]). This analysis was carried out in triplicate.

Cyclization trypsin/NGS assay—Lib2. mTG-NNK₄ & Lib3. mTG-NNK₆ cyclization libraries. mTG-NNK₄ and mTG-NNK₆ plinked RNA were carried out in similar fashion. Translation occurred (in triplicate) with a 19AA amino acid mix lacking methionine. The translation was supplemented with 50 mM biotin-Phe-initiator tRNA (solubilized in 1 mM NaOAc pH5.2). An 8.0 mL reaction was prepared, translated, and transcribed as stated above. Once complete the fusions were purified using anti-HA magnetic beads at a concentration of 4 mL bead slurry to 1 mL of IVT. This mixture was then diluted 10× from the original translation volume (e.g., 80 mL) and incubated while rotating at RT for 0.5 h. Once complete, the beads were washed 3× with 1×TBST (50 mM Tris-HCl pH 8.0, 200 mM NaCl, 0.1% TWEEN® 20) and brought up 80 mL of 1×TBST. The sample was then split into equal portions (40 μL), concentrated, and then brought back up in 1×TBST supplemented with or without 4 μM mTG (40 μL total solution). This slurry was mixed at 37° C. for 1 h on a benchtop shaker. After incubation, both samples were washed with 1×TBST, to remove mTG from solution, then resuspended with 20 mL (0.5× volume) of 2×TBST and 20 μL of 4 mg/mL HA peptide (to elute the fusion from the anti-HA bead). This was carried out at RT for 1 h. The final elution volume for each portion was 40 mL. Each sample (both treated or untreated with mTG) was then further split into equal portions (20 μL). One sample was digested with 100 nM trypsin (prepared as reported above) in a final reaction volume of 50 μL (1×TBST). The other sample was left untreated but diluted to 50 μL in 1×TBST. All samples were incubated at 4° C. for 0.5 h while rotating. Once complete magnetized Streptavidin beads (4 mL of streptavidin beads/1 mL original translation reaction) were added to each sample followed by 2× volume of 1×TBST (to dilute trypsin). Samples were incubated while rotating at 4° C. for 0.5 h then washed 3× with 1×TBST. After the final wash each sample was brought up in 1×PCR buffer (10 mM Tris-HCl pH 9.0, 50 mM KCl, and 0.1% TRITON X-100) and incubated at 95° C. for 5 mins to dissociate cDNA from each fusion. The supernatant was collected and the amount of recovered cDNA was determined by qPCR (see below). Recoveries were analyzed as a function of their corresponding non-digested sample. Isolated cDNA was PCR amplified based on the CT value from the qPCR results using standard Q5 polymerase and primers (P3, P9—annealing temperature of 57° C.). Once sufficient amplification was observed then the DNA was crashed out using a 0.1× (v/v) of 3M NaCl and 2× (v/v) of 100% ethanol. This was spun down at 15000 rpm for 10 mins, supernatant removed, washed with 70% EtOH and then allowed to dry. The (+) mTG and (+) trypsin samples were sent for NGS analysis (below)

B7-H3 selection. A 70 μL in vitro translation (IVT) reaction which contained plinked RNA for both mTG-NNK₆ and mTG-NNK₉ was prepared, translated, and transcribed as above. Once complete the fusions were purified using anti-HA magnetic beads at a concentration of 4 iL bead slurry to 1 uL of IVT. This mixture was then diluted 10× from the original translation volume (e.g., 700 mL), incubated while rotating at RT for 0.5 h, then washed 3× with 1× B7-H3 selection buffer (50 mM Tris-HCl pH 8.0, 150 mM NaCl, 0.2 mM CaCl₂, 0.05% TWEEN® 20). The fusion bound-HA beads were brought up in 200 μL of 2× selection buffer and then 200 μL of HA synthetic peptide (prepared as a 4 mg/mL solution in MQ-H₂O) was added. This mixture was rotated at RT for 1 h and upon completion the supernatant, containing purified fusion (400 μL), was collected. To this solution mTG (50 μM) and 1× selection buffer was added to a final concentration of 4 μM of mTG in solution (final volume 450 μL). This was incubated at 37° C. for 1 h. During incubation B7-H3 was loaded onto SA beads at a concentration of 2 μL of SA beads/1 pmol of B7-H3. This conversion was used to set up the selection against 0.2 μM B7-H3 in 450 μL total solution. To prepare, the requisite amount SA beads (180 μL) were aliquoted and washed with 1×TBST+CaCl₂). Then biotinylated B7-H3 was added for a final concentration of 0.2 μM in 450 μL. This was rotated at 4° C. for 15 mins. Then excess biotin (25 μL of 0.5M in MQ-H₂O) was added and the solution was incubated for an additional 15 mins—this step was to block any unreacted SA. Once complete the solution was removed from the beads and the fusion solution from the mTG reaction was added to the B7-H3-SA mixture. This was incubated while rotating at 4° C. for 0.5 h. After incubation the SA beads were washed 3× with 1× selection buffer. On the final wash the SA beads were resuspended into 10 mM Tris-Cl pH8.3, 50 mM KCl, 0.1% TRITON X-100 (1×PCR Buffer). To isolate the cDNA the SA bead solution was heated at 95° C. for 5 mins and the supernatant collected. The amount of recovered cDNA was determined by qPCR and analyzed as a function of the HA purified input amount. The cDNA was amplified based on the CT value from the qPCR results using standard Q5 polymerase protocol and primers (P3, P9—annealing temperature of 57° C.). Once sufficient amplification was observed then the DNA was crashed out using a 0.1× (v/v) of 3M NaCl and 2× (v/v) of 100% ethanol. This was spun down at 16000×g for 10 mins, supernatant removed, washed with 70% EtOH and then allowed to dry. A 20 μL transcription was set up using standard T7 RNA polymerase (NEB) to prepare material for the second and subsequent rounds.

isolated RNA from the first round of selections was then Plinked (as stated above) and carried forth into subsequent rounds. A 5 μL IVT reaction was prepared, translated, and transcribed as above. This was then HA purified at 4 μL of HA bead slurry to 1 μL of original IVT input (e.g., 20 μL of anti-HA beads to 5 μL of IVT input). This mixture was then diluted 10× from the original translation volume (e.g., 50 mL) and incubated while rotating at RT for 0.5 h. Once complete, the beads were washed 3× with 1× B7-H3 selection buffer (50 mM Tris-HCl pH 8.0, 150 mM NaCl, 0.2 mM CaCl₂, 0.05% TWEEN® 20). The fusion bound-HA beads were brought up in 25 μL of 2× B7-H3 selection buffer and then 25 μL of HA synthetic peptide (prepared as a 4 mg/ml solution in MQ-H₂O) was added. This mixture was rotated at RT for 1 h and upon completion the supernatant was collected, containing purified fusion (50 μL). To this solution mTG (50 μM) and 1× TBST+CaCl₂ was added to a final concentration of 4 μM of mTG in solution. This was incubated at 37° C. for 1 h. Next, negative selections against SA beads were performed to remove background binders—this was carried out for all rounds after the initial round. Three background selections were carried out at an equal volume of SA beads to input IVT (e.g., 5 μL of IVT to 5 μL of SA bead). To prepare 15 μL of SA beads were aliquoted, washed with 1× B7-H3 selection buffer, and then split into equal portions. To one portion biotin was added to a final concentration of 10 mM. Both portions were rotated at 4° C. for 0.5 h, then washed, resuspended in 1× B7-H3 selection buffer, and combined (final volume of 15 μL). This sample was then aliquoted into 3×5 μL portions. The HA purified and mTG treated fusion was then placed over these beads, incubated while rotating at 4° C. for 0.5 h, then removed. This process was repeated for every background selection. On the final background selection, after the fusion solution was removed, the beads were washed 3× with 1× B7-H3 selection buffer and then suspended in 1×PCR buffer. The sample was heated at 95° C. for 5 mins and the supernatant (containing bead binding cDNA) was collected for qPCR analysis. After screening through background selections, the library was incubated against 0.2 mM B7-H3 bound to SA (prepared as above but scaled down for this selection). This was carried out at 4° C. for 0.5 h, washed 3× with 1× B7-H3 selection buffer, and then suspended in 1×PCR. The cDNA was eluted by heating at 95° C. and collecting the supernatant. qPCR analysis, PCR amplification, and subsequent round RNA transcription was carried out as described in round 1. This process was repeated until sufficient enrichment was observed.

Selection buffer for B7-H3 comprised of 1×PBS (prepared from 10× stock, Gibco 14200-075) supplemented with 0.1% TWEEN® 20. Selections were carried out until enrichment was observed. For this selection (B7-H3) we observed a spike in round 6 to 1.3% of the HA purified input. The material from round 6 was sent for NGS analysis.

qPCR analysis. The amount of recovered cDNA was quantified by qPCR analysis using an Applied Biosystems Viia7 qPCR Real-Time qPCR Instrument. Each sample was analyzed in a 10 μL solution of 1× SsoAdvanced Universal SYBR Green Supermix (Bio-Rad, 172-5271), 0.125 μM Forward and Reverse primers (P10, P11), 2 μL of respective sample, and diluted to the final volume with MQ-H₂O. qPCR standards were prepared by reverse transcription of a known quantity of RNA into cDNA, assumed 100% yield, and dilutions to 2e⁹, 2e⁸, 2e⁷, 2e⁶, 2e⁵, 2e⁴ molecules were carried out. Standards were prepared fresh for every experiment. The qPCR method was carried out by first heating at 50° C. for 2 mins followed by 10 min 95° C. incubation. Cycling was then carried out between 95° C. for 15 sec and 60° C. for 1 min, with cycler heating acceleration held at 1.6° C. between each step. A total of 40 cycles was carried out in each qPCR run. During each elongation step, SYBR green fluorescence was measured with ROX as a passive reference. Following the run, standard curves were generated and used to calculate cDNA quantities.

NGS sample preparation. Recovered DNA from the end of the mRNA display round was prepared for Azenta Amplicon EZ NGS analysis. A standard Q5 polymerase reaction was carried out for 15 cycles (using primers P12-13). After sufficient amplification was observed the sample was cleaned up using a standard column purification kit and the concentration determined by nanodrop. Then 500 μg were sent (in MQ-H₂O) for NGS. In house python scripts were then used to analyze the NGS data.

Microwave assisted solid phase peptide synthesis. All peptides were prepared using 50-100 mg of Rink Amide ChemMatrix resin (0.48 mmol/g) following standard fluorenylmethoxycarbonyl (Fmoc) protocols. Prior to synthesis the resin was swelled at RT on a miniblock, shaking at 550 rpm, for 1 h in a mixture of 1:1 dichloromethane (DCM) and dimethylformamide (DMF). The reaction vessel was then transferred to a Biotage Initiator+ALSTRA™ to facilitate linear peptide synthesis.

Synthesis was carried out by (i) Fmoc removal using excess 20% piperidine (in DMF) which is added to the vessel for 3 mins while stirring at RT. The vessel is drained, washed with DMF, and excess 20% piperidine is added and allowed to stir at RT for 10 mins. The reaction is drained and washed again with DMF four times. (ii) Amino acid coupling is carried out on the deprotected resin by addition of the Fmoc-AA-OH (5.0 eq, 0.05M in DMF), O-(1H-6-Chlorobenzotriazole-1-yl)-1,1,3,3-tetramethyluronium hexafluorophosphate (HCTU) (5.0 eq, 0.05M in DMF), and n,n-diisoproplyethylamine (DIPEA) (10 eq, 1.0M in DMF). The suspension is mixed while heating at 75° C. for 5 mins. After coupling complete the vessel is then washed with DMF four times. This process is repeated until the full linear peptide is prepared. All amino acids were protected with standard acid labile protecting groups. For selective lactam bridge formation on B7-H3 peptides fmoc-glutamic acid 5-(2-phenylisopropyl) ester and fmoc-lysine(mTT)-OH were incorporated into the linear synthesis.

Selective lactam bridge formation B7-H3 peptides synthesis. The formation of both disulfide and lactam bridge was carried out on resin. The resin bound peptide was swelled in 1:1 DCM/DMF for 0.5 h prior to modification. To the resin 1 mL of deprotection solution (0.1N HCl and 1% TIPS in 100% hexafluoroisopropanol (HFIP)) was added to the resin and mixed for 1 min at RT (Vinogradov, et al., J. Am. Chem. Soc. 2019, 141:4167-4181; Huang, et al., Chem. Rev. 2019, 119:10360-10391). The solution was drained and an additional 1 mL of the deprotection solution was added and incubated 1 min. Then the mixture was drained and washed with DMF/DCM. Next, to induce disulfide formation, a solution containing 3 eq of I₂ was added in 100% methanol and incubated at RT for 10 mins. Following incubation the resin was washed briefly with 100% methanol and then DMF/DCM. To form the lactam bridge a solution of 10/10 eq PyBOP/HOBt/DIPEA was added in a 10/10/20 eq. This was mixed at RT for 20 mins, washed, and repeated 1×. Finally, 20% Piperidine in DMF was added to remove the final fmoc group. Then 10/10 eq of Acetic Anhydride and DIPEA in DMF was added and mixed at RT for 10 mins.

Global deprotection and cleavage from resin. After synthesis was complete the resin was dried and added to a clean 15 mL conical tube. To this, a solution of 95/2.5/2.5 eq Trifluoracetic acid/Triisopropylsilane/H₂O was added. This was mixed thoroughly at 37° C. for 1 h. If methionine was present in the peptide the resin was incubated at −80° C. until frozen. Then 15 eq of ammonium iodide was added and the solution was rotated at 4° C. until thawed. Once thawed the solution was filtered and the flow thorough evaporated with N₂. Then 100% diethyl ether was added to the crude peptide and the solution was spun down at 16,000×g to pellet the crashed-out peptide. The peptide was dried and stored at −80° C. until purification.

mTG in vitro reaction—B7-H3 peptide B1.1 analysis. A solution containing 60 μM linear B1.1 (from DMSO stock) was prepared in 50 mM Tris-Cl pH 8.0, 0.2M NaCl. To this solution mTG was added to a final concentration of 30 uM. This was incubated at 37° C. for 1 h. Then 1× volume of cold methanol was added, incubated on ice for 10 mins, followed by centrifugation at 16,000×g for 10 mins to crashout the enzyme, The supernatant was isolated and analyzed by LCMS. This was compared by retention time against synthetic B1.1.

Circular dichroism (CD) analysis. Lyophilized peptides (powder) were initially dissolved in 1:1 ACN/H₂O. The concentration was determined by nanodrop using the molecular weight and molar absorptivity for each peptide. Then each solution was diluted down to 120 μM using 10 mM Tris-H₂SO₄ pH 8.0. The final ACN concentration was 4%. CD spectra was acquired using an (Jasco J816 CD spectrometer) Measurements were acquired with taken every 1.0 nm at scanning speed of 20 nm/min from wavelengths of 250 nm to 200 nm. A high precision cell with 1 mm light path (Hellma Analytics, Art. No. 1101-1-40) was used to house the samples. Three spectra acquisitions were taken for each sample. A blank comprised of 4% ACN in 10 mM Tris-H₂SO₄ pH8.0 was first run followed by each peptide (linear, i,i+4, and i,i+7). The blank values were subtracted from each sample. The degree of ellipticity, [θ] in deg cm² dmol⁻, was calculated as follows:

$$[ θ ] = \frac{( 100 \times CD signal ( nm ) )}{( Concentration mM ) ( \# Amino Acids ) ( path length ( cm )}$$

Surface Plasmon Resonance (SPR) analysis. Binding kinetics between our selected peptide (and variants) and biotinylated B7-H3 (B7-H3b) were assessed through SPR analysis using a BIACORE 8K SPR system (GE Healthcare). The running buffer was 1×PBS pH7.5, 0.05% TWEEN® 20 and all analysis was carried out at 25° C. B7-H3b was loaded onto a NeutrAvidin SPR Chip (Series S Sensor Chip NA, 29407997, Cytiva) at a flow rate of 30 ug/min to a surface density of 1000-1500 response units. Analyte was then injected over a series of five concentrations and quantified by BIACORE single-cycle kinetics method. For our selected peptide (B1.1) and the Met9Nle variant a range of 7.5, 15, 30, 60, 120 nM were run. For the linear variant concentrations of 50, 100, 200, 400, 800 nM were run. Binding sonograms were generated using a standard 1:1 binding kinetics model and kinetics constants determined using standard BIACORE evaluation software.

Modeling—Helicity analysis. Propensities to form an a-helix were calculated using the Agadir model (Lacroix, et al. Journal of Molecular Biology, 1998, 284(1):173-191) for 5,000 randomly selected cyclized peptides and for 5,000 random peptide sequences from our NNK₆ lysine cyclization NGS dataset (Lib. 3). The two parameters of the model, temperature and pH, were set to respectively 298 K and 7.4. Probability density functions (PDF) of the calculated propensities were calculated separately for both data sets. Both PDF are identical, suggesting that the calculated propensity to form a helix is not predictive of the peptide cyclization by mTG.

### Example 2. Development of B7-H3 Ligands for Drug Delivery

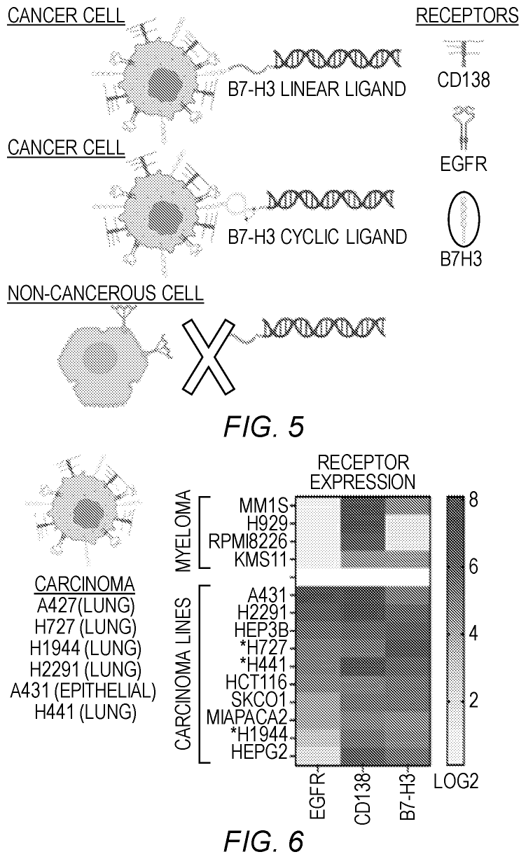

FIG. 5 shows a schematic of B7-H3-targeting ligand comprising an exemplary oligonucleotide therapeutic, which can include B7-H3 linear and cyclic targeting ligands. Additional cargo payloads can be conjugated to the target-ligand as therapeutics for delivery to solid tumors. Example linear and cyclic ligands described herein include B2.1, JP3, JP5 and B1.1. The ligands can be labeled with TAMRA fluorophore for visualization, making the targeting ligands useful in imaging modalities. Receptor distribution of B7-H3 receptor and heatmap of receptor expression in myeloma and carcinoma cell lines shows the broad expression of B7-H3 in cell lines. (FIG. 6).

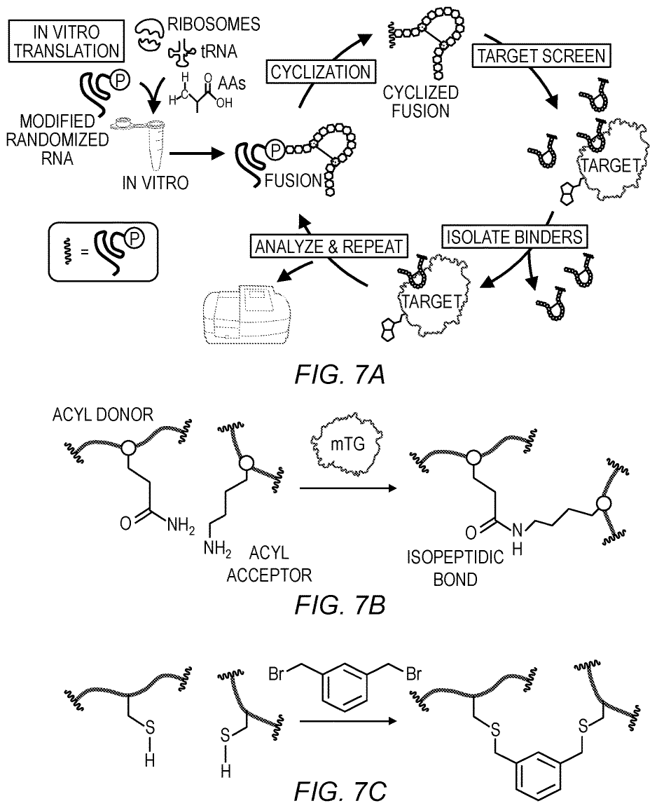

FIG. 7A provides the general mRNA display schematic. Randomized mRNA (modified on the 3′ end by attaching a bulky puromycin compound) is translated into its corresponding peptide using in vitro translation (IVT) components. When the ribosome comes into contact with the puromycin on the RNA strand it stalls translation. During this time, the puromycin reacts with the C-terminus of the corresponding peptide to create a physical linkage between the mRNA and peptide (also referred to as a peptide-RNA fusion or just fusion). This links together the phenotype (peptide) to the genotype (randomized RNA sequence) and enables identification through sequencing of the genetic tag. These fusions can then be modified (in this case to form macrocyclic peptides—see FIG. 7B or FIG. 7C) to increase the interaction capabilities of the randomized peptide libraries. Once modified, the fusions are then panned against an immobilized target of interest. After incubation, the target is washed (to remove non-binders) and any fusions that stick to the target are isolated. The genetic tag can be recovered and analyzed by qPCR and sequencing. Further rounds of mRNA display can be carried out by using the isolated genetic material as the starting point for generating new peptide-mRNA libraries (filtered to specifically interact with the target of interest). This can be carried out until enrichment is observed (defined as a high recovery of material binding to the target over what goes into the selection).

Microbial transglutaminase (mTG)-mediated cyclization is depicted in FIG. 7B. mTG forms a lactam bridge (isopeptidic bond) between glutamine and lysine residues. If these residues are present in the same peptide mTG can then be used to form lactam bridged macrocyclic peptides. The fusions were design to include a fixed glutamine and lysine bonds flanking the randomized amino acid sequences. Incubation of these fusions with mTG will induce lactam bridge formation—thus installation of a macrocycle around the randomized amino acid sequence. This process was used for the enzymatic/mTG cyclization and the B1.1 hits.

Dibromoxylene-mediated thioether cyclization is depicted in FIG. 7C. Free thiols (found in cysteine amino acid residues) will react with alkyl-halides to form a thioether bond. Crosslinking reagents (such as dibromoxylene) can react with cysteine residues to form thioether linked macrocyclic peptides. Libraries were designed to have two cysteine residues flanking the randomized amino acid sequences. Once the library is generated the fusions are incubated with dibromoxylene to crosslink the two fixed cysteine residues and form the macrocycle. This process was used for the thioether linked macrocycles and JP3 and JP5 hits.

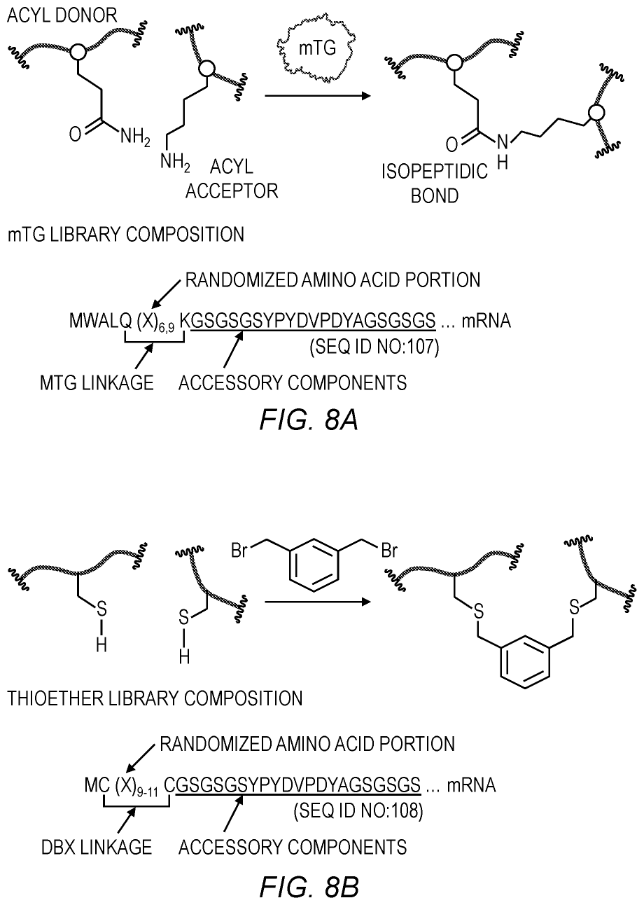

The library design for mRNA display selection is depicted in FIGS. 8A-8B. The library is a collection of randomized peptide sequences to be screened against the target of interest, including libraries of 6 and 9 randomized amino acid residues for mTG libraries (FIG. 8A) and libraries of 9, 10, and 11 randomized amino acid sequences for DBX libraries (FIG. 8B).

### Example 3. MTG-Mediated B7-H3 Selection

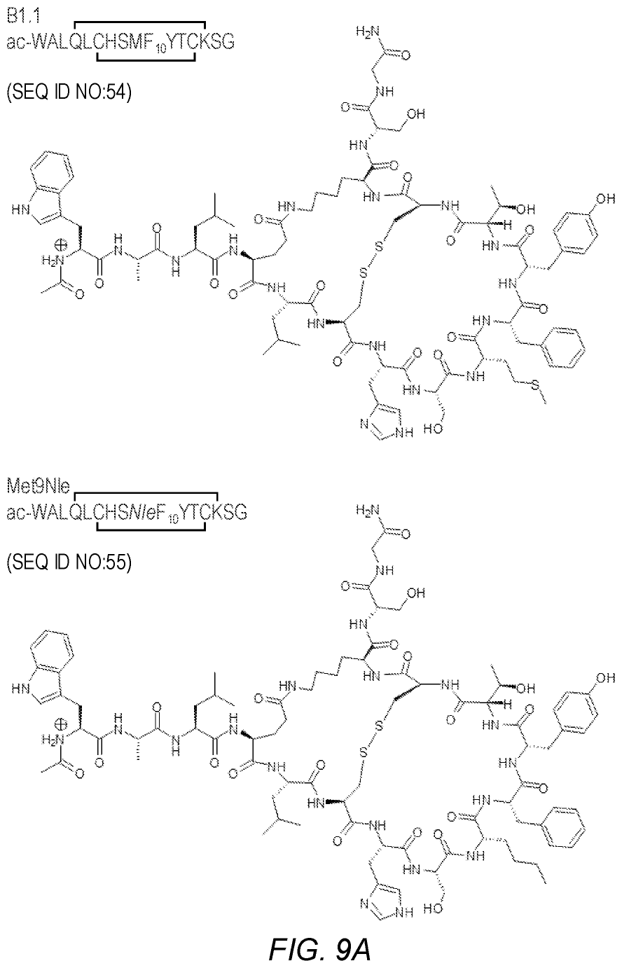

The selection process for identifying binders against B7-H3 was undertaken with mTG mediated B7-H3 selection. Peptide-mRNA fusions (in complex with cDNA) are generated through in vitro translation (mRNA display), as shown in FIG. 4A. These fusions are incubated with mTG to form a glutamine to lysine side chain macrocycle (lactam bridged macrocycle). These fusions are then incubated w/streptavidin immobilized B7-H3. The immobilized B7-H3 is then washed to remove any non-binders. Next the cDNA is isolated through heat elution of the immobilized B7-H3. This is analyzed as a function of cDNA going into the selection—through qPCR. The cDNA is used to regenerate the mRNA and repeat the process. The selections are carried out until enrichment is observed by qPCR (a high recovery of material that entered the selection is retained after incubation with immobilized B7-H3). Next generation sequencing (NGS) is used to identify the composition of peptide hits. Recovery is a measure of the material retained after the selection versus what goes into the selection. A background screen against selection components is carried out prior to the selection against B7-H3 to remove non-specific binders. Enrichment for the positive selection was observed in round 6, with qPCR results of the selection shown in FIG. 4B. NGS data of the selection hits shows one family type observed for this selection, with B1.1 ligand pursued for binding validation (FIG. 4D). An orthogonal validation confirms mTG is able to modify the linear precursor peptide into the cyclic hit as shown in FIG. 4E. Three peptides were prepared for Surface Plasmon Resonance analysis: the selected cyclic, disulfide peptide (B1.1), the linear variant, and a methionine to norleucine (Met9Nle) variant of the selected cyclic, disulfide peptide. Both cyclic peptides displayed low nM affinities against B7-H3 (Selected 43.5 nM, Met9Nle 46.9 nM) (FIG. 4F). The linear precursor peptide did not bind, detailing the need for the cyclic modifications. Structures of exemplary mTG mediated B7-H3 hit structures are shown in FIG. 9A, with conjugation sites at the N-terminal and C-terminal ends of the molecules identified in FIG. 9B. A general sequence of ac-WALQLCXXXF₁₀YTCKSG (SEQ ID NO:25) is identified (Table 5), with X amino acid residues X₇₋₉ variable amino acids. Q₄-K₁₄ form a lactam bridge and C₆-C₁₃ a disulfide bond. N-terminal capping, for example, acetylation, provides resistance to degradation. It may be the C-terminus is an optimal conjugation location because that targeting ligand peptide was selected with a C-terminal mRNA tag.

TABLE 5
Sequences for B1 family ligands cyclized by mTGase

| Hit | Full sequence | (−)N-term Methionine | Nle variant |
|---|---|---|---|
| B1.1 | acMWALQLCHSMFYTCKSG | acWALQLCHSMFYTCKSG | acWALQLCHSNleFYTCKSG |
| | (SEQ ID NO: 53) | (SEQ ID NO: 54) | (SEQ ID NO: 55) |
| B1.2 | acMWALQLCVSKYYTCKSG | acWALQLCVSKYYTCKSG | Na |
| small | (SEQ ID NO: 56) | (SEQ ID NO: 57) | |
| B1.2 | acMWALQLCVSKYYTCKSG | acWALQLCVSKYYTCKSG | Na |
| large | (SEQ ID NO: 58) | (SEQ ID NO: 59) | |
| B1.3 | acMWALQLCTSMFYTCKSG | acWALQLCTSMFYTCKSG | acWALQLCTSNleFYTCKSG |
| | (SEQ ID NO: 60) | (SEQ ID NO: 61) | (SEQ ID NO: 166) |
| B1.4 | acMWALQLCSSRFYTCKSG | acWALQLCSSRFYTCKSG | Na |
| | (SEQ ID NO: 62) | (SEQ ID NO: 63) | |
| B1.5 | acMWALQLCISRYYTCKSG | acWALQLCISRYYTCKSG | Na |
| | (SEQ ID NO: 64) | (SEQ ID NO: 65) | |
| B1.6 | acMWALQLCTSYWYTCKSG | acWALQLCTSYWYTCKSG | Na |
| | (SEQ ID NO: 66) | (SEQ ID NO: 67) | |

Bold = lactam bridge
Underline = disulfide
Ac = acetylation

### Example 4. Linear B7-H13 Ligands

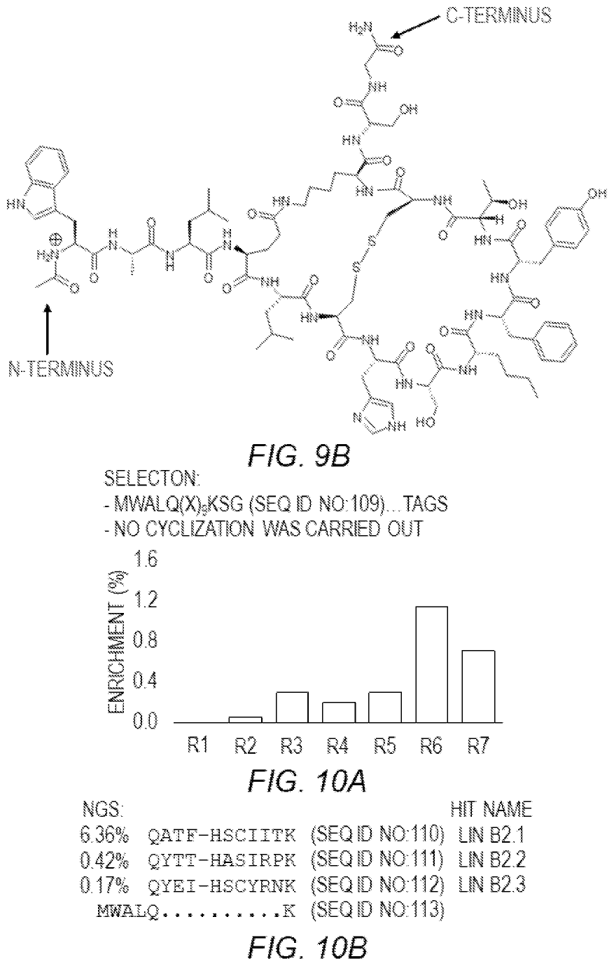

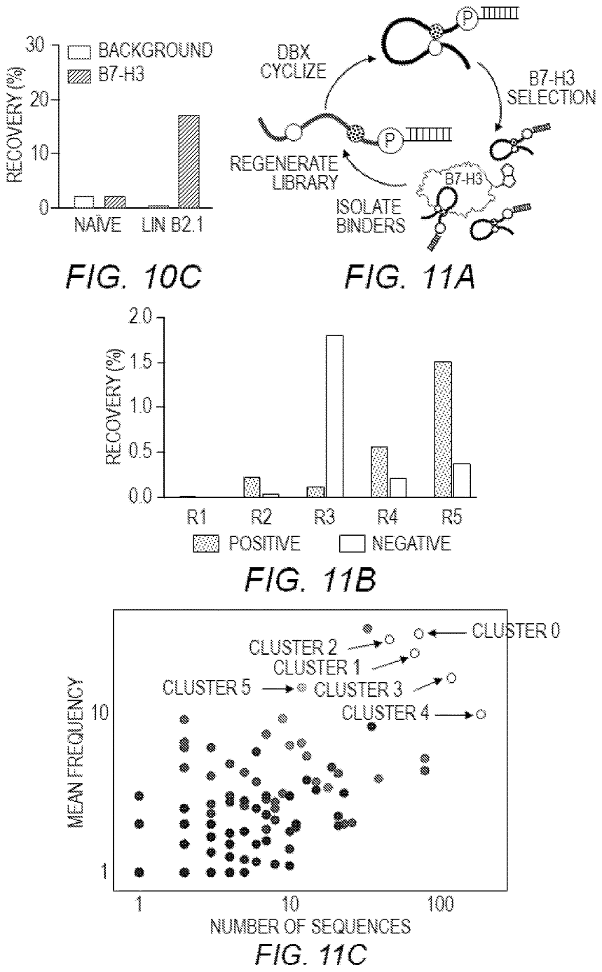

Results of a linear selection using the mTG-NNK₉ library are shown in FIGS. 10A-10C. No modifications, i.e., cyclization, were installed. FIG. 10A shows the selection results for the linear library against 137-13 over 7 rounds. Relevant NGS hits from the selection of linear sequences are shown in FIG. 10B. FIG. 10C shows a sequence validation assay for Lin B2.1, which monitors the recovery of a single sequence against B7-H3. High recoveries compared to background indicate specificity for the target (B7-H3). A naïve NNK₉ (no selection conducted) was run in parallel and did not enrich beyond background in this selection. Linear hit sequences are shown in Table 6.

TABLE 6
Sequences for B1 family ligands cyclized by mTGase

| Hit | Full sequence | (−)N-term Methionine |
|---|---|---|
| Lin | acMWALQAFTHSCIITKSG | acWALQAFTHSCIITKSG |
| B2.1 | (SEQ ID NO: 68) | (SEQ ID NO: 69) |
| Lin | acMWALQYTTHSCIRPKSG | acWALQYTTHSCIRPKSG |
| B2.2 | (SEQ ID NO: 70) | (SEQ ID NO: 71) |
| Lin | acMWALQYEIHSCYRNKSG | acWALQYEIHSCYRNKSG |
| B2.3 | (SEQ ID NO: 72) | (SEQ ID NO: 73) |

### Example 5. Dibromoxylene (DBX)-Mediated B7-H3 Selection

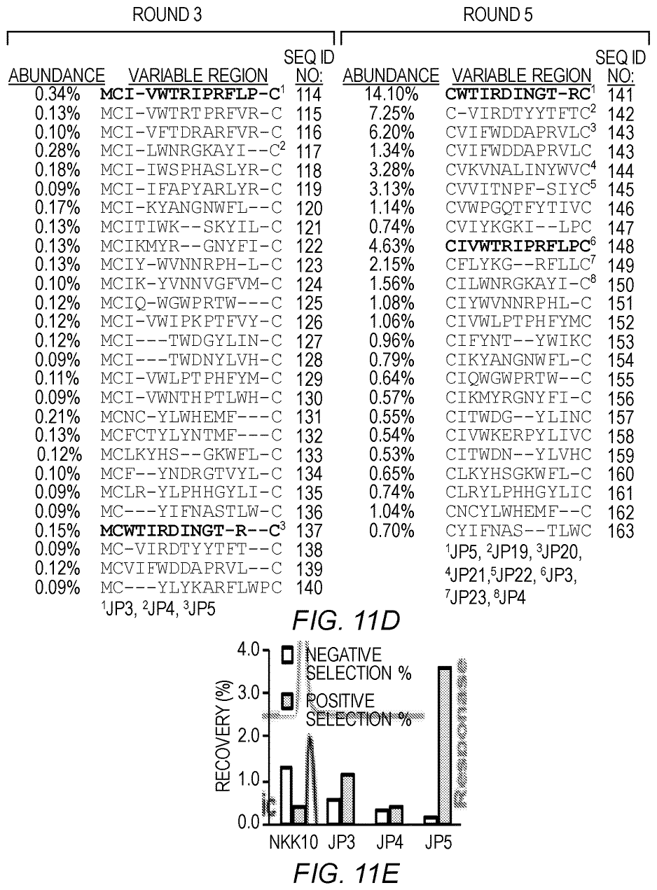

Selection studies with DBX identifies binders against B7-H3 by generating peptide-mRNA fusions (in complex with cDNA) through in vitro translation (mRNA display)(FIG. 11A). These fusions are incubated with DBX (dibromoxylene) to form a thioether macrocycle. These fusions are then incubated w/streptavidin immobilized B7-H3. The immobilized B7-H3 is then washed to remove any non-binders. Next, the cDNA is isolated through heat elution of the immobilized B7-H3. This is analyzed as a function of cDNA going into the selection—through qPCR. The cDNA is used to regenerate the mRNA and repeat the process. Selections are carried out until enrichment is observed by qPCR (a high recovery of material that entered the selection is retained after incubation with immobilized B7-H3). Next generation sequencing (NGS) is used to identify the composition of peptide hits. qPCR results are shown in FIG. 11B, with recovery as a measure of the material retained after the selection versus what goes into the selection. The analysis identified six clusters (0-5) of particular interest (FIG. 11C; Table 7). Linear hit sequences are shown in Table 8.

TABLE 7
DBX selection family analysis

| | Number of | | |
|---|---|---|---|
| Cluster Number | Sequences | Mean Frequency | Seed Frequency |
| 0 | 71 | 31.1 | 1887 |
| 1 | 67 | 23.7 | 1195 |
| 2 | 46 | 28.7 | 1188 |
| 3 | 117 | 16.5 | 1004 |
| 4 | 189 | 9.7 | 708 |
| 5 | 12 | 14.3 | 148 |

TABLE 8
DBX selection family sequences

| | | | | | |
|---|---|---|---|---|---|
| | | SEQ | Round 1 | | SEQ |
| | | ID | Occurrence | Variable | ID |
| ID | Sequence | NO: | (%) | Sequence | NO: |

Cluster 0

| | | | | | |
|---|---|---|---|---|---|
| 1 | MCIVWTRIPRFLPCGSGSGSYPY | 167 | 13.05 | IVWTRIPRFLP | 168 |
| | DVPDYAGSGSGS | | | | |
| 28 | MCIVWTRIPRFLPCGSGSGSYPY | 169 | 0.28 | IVWTRIPRFLP | 168 |
| | DVPDYVGSGSGS | | | | |
| 31 | MCIVWTRIPRFLPCGSGSGSYPY | 170 | 0.25 | IVWTRIPRFLP | 168 |
| | DVPDYAGSRSGS | | | | |
| 36 | MCIVWTYPPRLVPCGSGSGSYP | 171 | 0.22 | IVWTYPPRLVP | 172 |
| | YDVPDYAGSGSGS | | | | |
| 50 | MCIVWTRIPRFLPCGSGSGSYPY | 173 | 0.17 | IVWTRIPRFLP | 168 |
| | DVPDYSGSGSGS | | | | |
| 66 | MCIVWTRIPRFLPCGSGLGSYPY | 174 | 0.12 | IVWTRIPRFLP | 168 |
| | DVPDYAGSGSGS | | | | |
| 85 | MCIVWTRIPRFLPCGSGSGSYPY | 175 | 0.10 | IVWTRIPRFLP | 168 |
| | DVPDYARSGSGS | | | | |
| 89 | MCIVWTRIPRFLPCGSGSVSYPY | 176 | 0.10 | IVWTRIPRFLP | 168 |
| | DVPDYAGSGSGS | | | | |
| 97 | MCIVWTRIPRFLPCGSGSGLYPY | 177 | 0.08 | IVWTRIPRFLP | 168 |
| | DVPDYAGSGSGS | | | | |
| 110 | MCIVWTRIPRFLPCGSGSGSYPY | 178 | 0.08 | IVWTRIPRFLP | 168 |
| | DVPDYAWSGSGS | | | | |

Cluster 1

| | | | | | |
|---|---|---|---|---|---|
| 2 | MCIYWVNNRPHLCGSGSGSYPY | 179 | 8.27 | IYWVNNRPHL | 180 |
| | DVPDYAGSGSGS | | | | |
| 9 | MCIYWTGDVPRLCGSGSGWYP | 181 | 1.58 | IYWTGDVPRL | 182 |
| | YDVPDYAGSGSGS | | | | |
| 68 | MCIYWTGNRARICGSGSGSYPY | 183 | 0.12 | IYWTGNRARI | 184 |
| | DVPDYAGSGSGS | | | | |
| 82 | MCIYWTSGRARICGSGSGSYPY | 185 | 0.10 | IYWTSGRARI | 186 |
| | DVPDYAGSGSGS | | | | |
| 101 | MCIYWTGDVPRLCGSGSGSYPY | 187 | 0.08 | IYWTGDVPRL | 182 |
| | DVPDYAGSGSGS | | | | |
| 114 | MCIYWVNNRPHLCSSGSGSYPY | 188 | 0.07 | IYWVNNRPHL | 180 |
| | DVPDYAGSGSGS | | | | |
| 136 | MCIYWVNNRPHLCGSGSGSYPY | 189 | 0.06 | IYWVNNRPHL | 180 |
| | DVPDYVGSGSGS | | | | |
| 157 | MCIYWVNNRPHLCGSGSGSYPY | 190 | 0.06 | IYWVNNRPHL | 180 |
| | DVPDYARSGSGS | | | | |
| 207 | MCIYWVTNRPFLCGSGSGSYPY | 191 | 0.04 | IYWVTNRPFL | 192 |
| | DVPDYAGSGSGS | | | | |
| 215 | MCIYWVNNRPHLCGSGSGSYPY | 193 | 0.04 | IYWVNNRPHL | 180 |
| | DVPDYAWSGSGS | | | | |

Cluster 2

| | | | | | |
|---|---|---|---|---|---|
| 3 | MCIFIDDLYGMRCGSGSGSYPY | 194 | 8.22 | IFIDDLYGMR | 195 |
| | DVPDYAGSGSGS | | | | |
| 26 | MCWTIRDINGTRCGSGSGSYPY | 196 | 0.30 | WTIRDINGTR | 197 |
| | DVPDYAGSGSGS | | | | |
| 64 | MCVEIWDISGYRCGSGSGSYPY | 198 | 0.14 | VEIWDISGYR | 199 |
| | DVPDYAGSGSGS | | | | |
| 162 | MCIFIDDLYGMRCGSGSGSYPY | 200 | 0.05 | IFIDDLYGMR | 195 |
| | DVPDYVGSGSGS | | | | |
| 189 | MCIFIDDLYGMRCGSGSWSYPY | 201 | 0.04 | IFIDDLYGMR | 195 |
| | DVPDYAGSGSGS | | | | |
| 241 | MCIFIDDLYWMRCGSGSGSYPY | 202 | 0.04 | IFIDDLYWMR | 203 |
| | DVPDYAGSGSGS | | | | |
| 373 | MCIFIDDLYGMRCGSGSGSYPY | 204 | 0.02 | IFIDDLYGMR | 195 |
| | DVPDYAGSVSGS | | | | |
| 386 | MCIFIDDLYGMRCGSGSGSYPY | 205 | 0.02 | IFIDDLYGMR | 195 |
| | YVPDYAGSGSGS | | | | |
| 392 | MCIFIDDLYGMRCGSGSVSYPY | 206 | 0.02 | IFIDDLYGMR | 195 |
| | DVPDYAGSGSGS | | | | |
| 451 | MCIFIDDLYGMRCGSGSGSYPY | 207 | 0.01 | IFIDDLYGMR | 195 |
| | DVPDYAWSGSGS | | | | |

Cluster 3

| | | | | | |
|---|---|---|---|---|---|
| 4 | MCILPWHGQFLICGSGSGSYPYD | 208 | 6.94 | ILPWHGQFLI | 209 |
| | VPDYAGSGSGS | | | | |
| 7 | MCITFYRGNFVICGSGSGSYPYD | 210 | 3.47 | ITFYRGNFVI | 21 |
| | VPDYAGSGSGS | | | | |
| 12 | MCIYPYRGAFLLCGSGSGSYPYD | 212 | 0.81 | IYPYRGAFLL | 213 |
| | VPDYAGSGSGS | | | | |
| 20 | MCILPTTGGFYLCGSGSGSYPYD | 214 | 0.44 | ILPTTGGFYL | 215 |
| | VPDYARSGSGS | | | | |
| 54 | MCVFPYRGKFFICGSGSGSYPYD | 216 | 0.17 | VFPYRGKFFI | 217 |
| | VPDYAGSGSGS | | | | |
| 100 | MCIRTYYGQWLLCGSGSGSYPY | 218 | 0.08 | IRTYYGQWLL | 219 |
| | DVPDYAGSGSGS | | | | |
| 121 | MCIVPYLGAFLICGSGSGSYPYD | 220 | 0.07 | IVPYLGAFLI | 221 |
| | VPDYAGSGSGS | | | | |
| 139 | MCIKPYRNNFLICGSGSGSYPYD | 222 | 0.06 | IKPYRNNFLI | 223 |
| | VPDYAGSGSGS | | | | |
| 165 | MCILPFNGLYFLCGSGSGSYPYD | 224 | 0.05 | ILPFNGLYFL | 225 |
| | VPDYAGSGSGS | | | | |
| 166 | MCLLQWHGGFYLCGSGSGSYPY | 226 | 0.05 | LLQWHGGFYL | 227 |
| | DVPDYAGSGSGS | | | | |

Cluster 4

| | | | | | |
|---|---|---|---|---|---|
| 6 | MCIKVIGSRFYLCGSGSGSYPYD | 228 | 4.90 | IKVIGSRFYL | 229 |
| | VPDYAGSGSGS | | | | |
| 8 | MCIRYVGGHFYLCGSGSGSYPY | 230 | 2.31 | IRYVGGHFYL | 231 |
| | DVPDYAGSGSGS | | | | |
| 13 | MCIRLESGKFYLCGSGSGSYPYD | 232 | 0.70 | IRLESGKFYL | 233 |
| | VPDYAGSGSGS | | | | |
| 15 | MCIKYILGSWYLCGSGSGSYPY | 234 | 0.58 | IKYILGSWYL | 235 |
| | DVPDYAGSGSGS | | | | |
| 19 | MCIKVNGPHFYLCGSGLGSYPY | 236 | 0.45 | IKVNGPHFYL | 237 |
| | DVPDYAGSGSGS | | | | |
| 35 | MCIRFTRAGFLLCGSGSGSYPYD | 238 | 0.23 | IRFTRAGFLL | 239 |
| | VPDYAGSGSGS | | | | |
| 39 | MCIRYVLNNWYLCGSGSGSYPY | 240 | 0.21 | IRYVLNNWYL | 241 |
| | DVPDYAGSGSGS | | | | |
| 46 | MCIRFVSGYYYLCGSGSGSYPY | 242 | 0.19 | IRFVSGYYYL | 243 |
| | DVPDYAGSGSGS | | | | |
| 58 | MCIRLMQGHFYLCGSGSGSYPY | 244 | 0.16 | IRLMQGHFYL | 245 |
| | DVPDYAGSGSGS | | | | |
| 70 | MCIALLKGRFYLCGSGSGSYPY | 246 | 0.12 | IALLKGRFYL | 247 |
| | DVPDYAGSGSGS | | | | |

Cluster 5

| | | | | | |
|---|---|---|---|---|---|
| 11 | MCWKITDVYYVICGSGSGSYPY | 248 | 1.02 | WKITDVYYVI | 249 |
| | DVPDYAGSGSGS | | | | |
| 178 | MCWSIWDIHGVRCGSGSGSYPY | 250 | 0.05 | WSIWDIHGVR | 251 |
| | DVPDYAGSGSGS | | | | |
| 270 | MCIKLLNGYFIICGSGSGSYPYD | 252 | 0.03 | IKLLNGYFII | 253 |
| | VPDYAGSGSGS | | | | |
| 435 | MCWKITDVYYVICGSGSGSYPY | 254 | 0.01 | WKITDVYYVI | 249 |
| | DVPDYVGSGSGS | | | | |
| 535 | MCWKITEVYYVICGSGSGSYPY | 255 | 0.01 | WKITEVYYVI | 256 |
| | DVPDYAGSGSGS | | | | |
| 634 | MCWKITDVYYVICGSGSGSYPY | 257 | 0.01 | WKITDVYYVI | 249 |
| | DVPDYARSGSGS | | | | |
| 1182 | MCWKITDVYYVICGSGSGSYPY | 258 | 0.01 | WKITDVYYVI | 249 |
| | DVPDYAGRGSGS | | | | |
| 1186 | MCIRFVSVYYYLCGSGSGSYPY | 259 | 0.01 | IRFVSVYYYL | 260 |
| | DVPDYAGSGSGS | | | | |
| 1202 | MCWKITDVYYVICGSGSGSYPY | 261 | 0.01 | WKITDVYYVI | 249 |
| | DVPDYAGSGSGS | | | | |
| 1733 | MCWKITDVYYVICGNGSGSYPY | 262 | 0.01 | WKITDVYYVI | 249 |
| | DVPDYAGSGSGS | | | | |

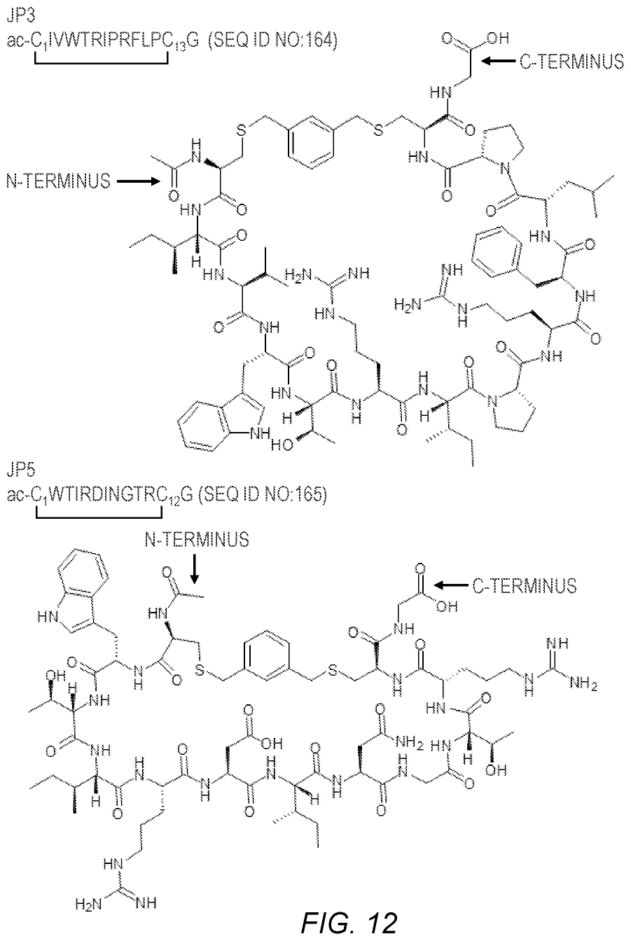

A background screen against selection components is carried out prior to the selection against B7-H3 to remove non-specific binders. FIG. 11D includes sequences from NGS data with named sequences evaluated for binding validation. The validation assay in FIG. 11E identified JP3 and JP5 as strong B7-H3 binders. Exemplary DBX hit structures are shown in FIG. 12, with points of cargo attachment designated with arrows.

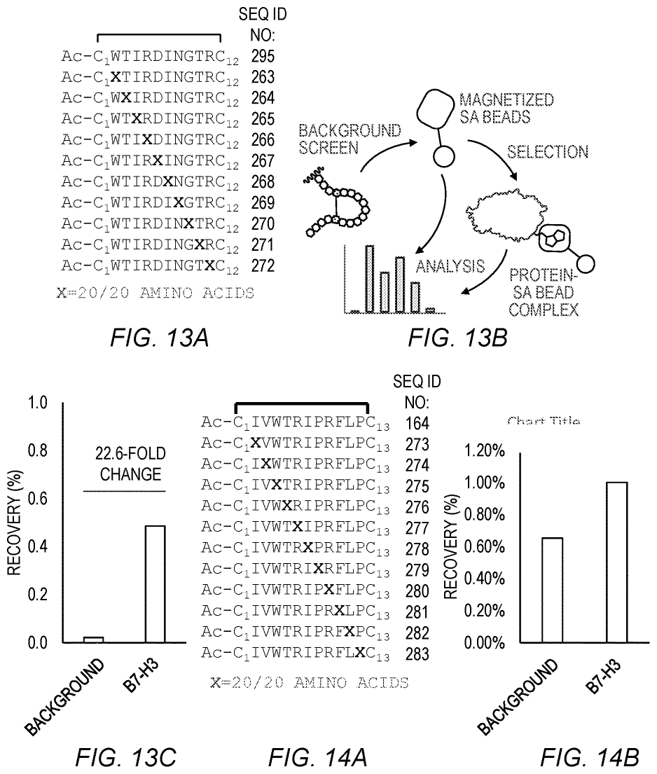

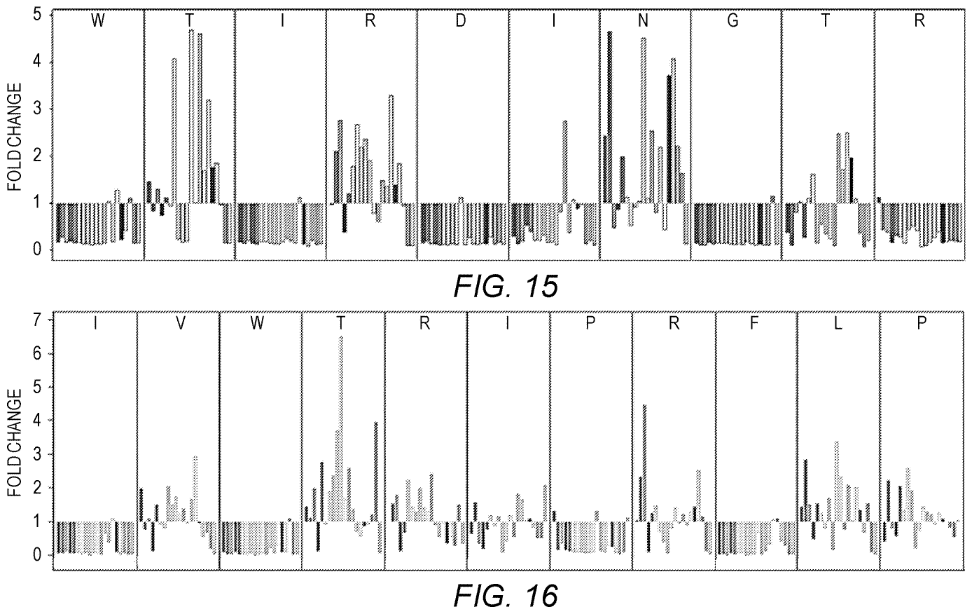

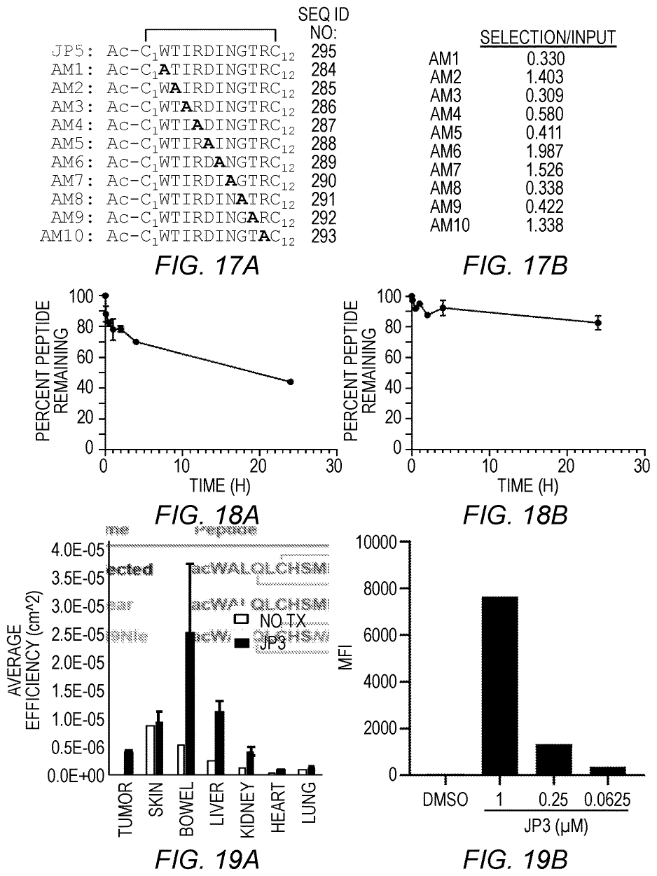

A single variant library of JP5 (FIG. 13A) and JP3 (FIG. 14A) was carried out. Each residue was mutated to include potentially 20/20 canonical amino acids. All library members were screened first against background components (magnetic beads) to remove nonspecific binders and then against biotinylated B7-H3 (FIG. 13B). The results were analyzed by qPCR which showed much higher recovery when the library was incubated with B7-H3 (FIG. 13C and FIG. 14B). The recovered material from the B7-H3 selection of JP5 and JP3 was sent for NGS (FIG. 15 and FIG. 16, respectively). In addition, JP5 was subjected to an alanine mutant scan (FIG. 17A). All library members were screened first against background components (magnetic beads) to remove nonspecific binders and then against biotinylated B7-H3 (see FIG. 13B). The results were analyzed by NGS which showed relative enrichment values for each Alanine Mutant (AM; FIG. 17B). AMs that were less enriched in the selection were chosen for synthesis as JP5 negative controls that exhibit similar molecular weight and physical properties as JP5. The selected AMs will be tested with surface plasmon resonance (SPR) to assess binding affinity.

Serum stability of JP3 and JP5 was assessed. Peptides (JP3 or JP5) were incubated in 50% fetal bovine serum (diluted in media) at 37° C. and samples were taken over a time course spanning 24 h. Samples were mixed with cold methanol to crash out serum protein, and the supernatant containing the peptides was collected and run on MS to determine the amount of peptide present at each time point. Both JP3 and JP5 showed high levels of serum stability, with ~40% of JP3, and 80% of JP5 remaining intact following incubation with serum at 37° C. for 24 h (FIGS. 18A-18B).

Results of in vivo evaluation of JP3 in mice inoculated with subcutaneous A427 (lung cancer) tumors are shown in FIG. 19A. Mice were dosed with JP3-TAMRA (16 nmol/mouse, 200 μL IV). Mice were sacrificed 4 h post injection, and tissues resected for IVIS imaging (to measure fluorescence). The tissue distribution displayed tumor accumulation, as well as accumulation in bowel, liver, and kidney. B7-H3 expressing lung cancer cells (H727) were treated with DMSO or varying concentrations (1 μM, 0.25 μM, 0.0625 μM) of JP3 conjugated with TAMRA via a PEG3 linker. Cells were treated for 2 hours, followed by washing and then prepared for FACS analysis. Cells were quantified for TAMRA wavelength signal to indicate binding of the ligand. FIG. 19B shows a dose-response by FACS for TAM/RA signal.

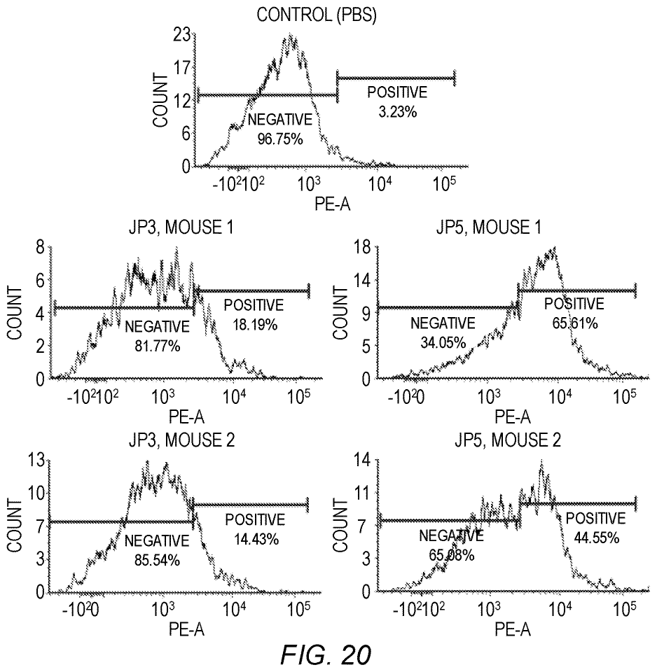

Uptake of JP3 and JP5 is shown in FIG. 20. Mice with subcutaneous H727 tumors (B7-H3 expressing tumor model) were dosed with 15 nm of JP3-TAMRA of JP5-TAMRA subcutaneously. Mice were sacrificed 3 h post injection, tumors resected, homogenized, and evaluated for peptide uptake in tumor cells (TAMRA signal). Mice treated with JP3 displayed an average of 16% of tumor cells positive for JP3 ligand, mice treated with JP5 displayed an average of 55% of tumor cells positive for JP5 ligand.

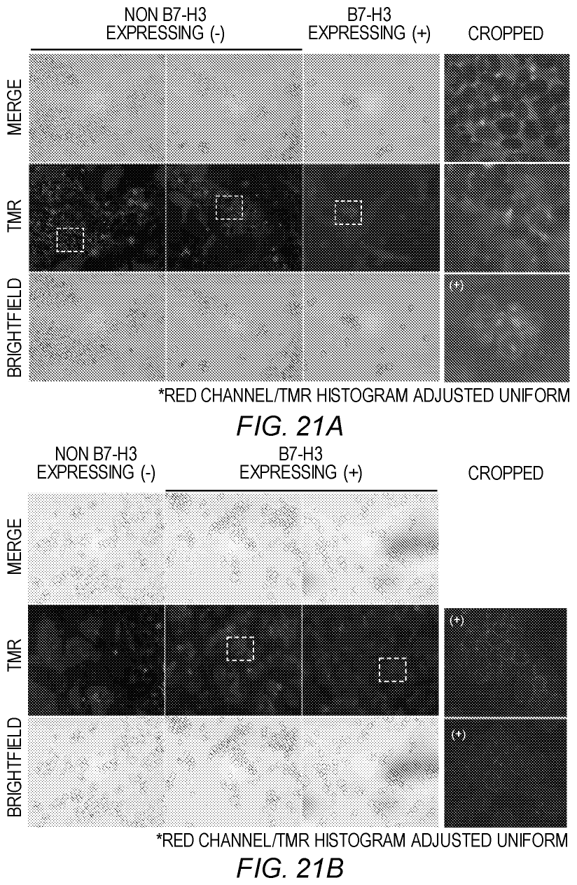

JP3 and JP5 uptake was measured in B7-H3 expressing Raji cells. Raji cells (which are negative for B7-H3 at baseline) were engineered to over-express murine B7-H3. Cells were incubated with JP3-PEG-TAMRA (FIG. 21A) or JP5-PEG-TAMRA (FIG. 21B) for 2 hours at 1 μM in concentration. Cells were then washed, cytospun and live imaging was conducted. Compared with Raji negative controls (left and middle columns), the Raji-murine B7-H3 expressing cells (right column) were observed to silhouette the cell surface, consistent with surface binding of the targeting ligand and to show uptake signal in endosomal like structures.

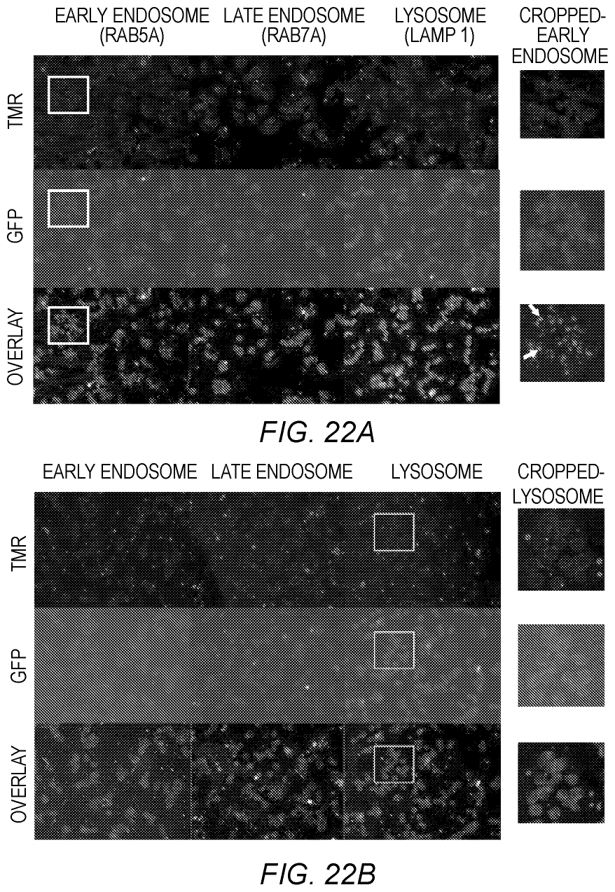

Live imaging of an example targeting ligand uptake in Raji cells expressing murine B7-H3 pre-incubated with GFP-tagged reporters for early (Rab5a) and late (Rab7a) endosomes or early (Lamp1) lysosomes is shown in FIG. 22A (JP3) and FIG. 22B (JP5). Co-localization of JP3 and Rab5a (FIG. 22A, far right column) was observed, indicative of receptor-mediated endocytosis. In FIG. 22B, predominantly cell surface localization of JP5 was observed with some rare evidence of endosomal uptake.

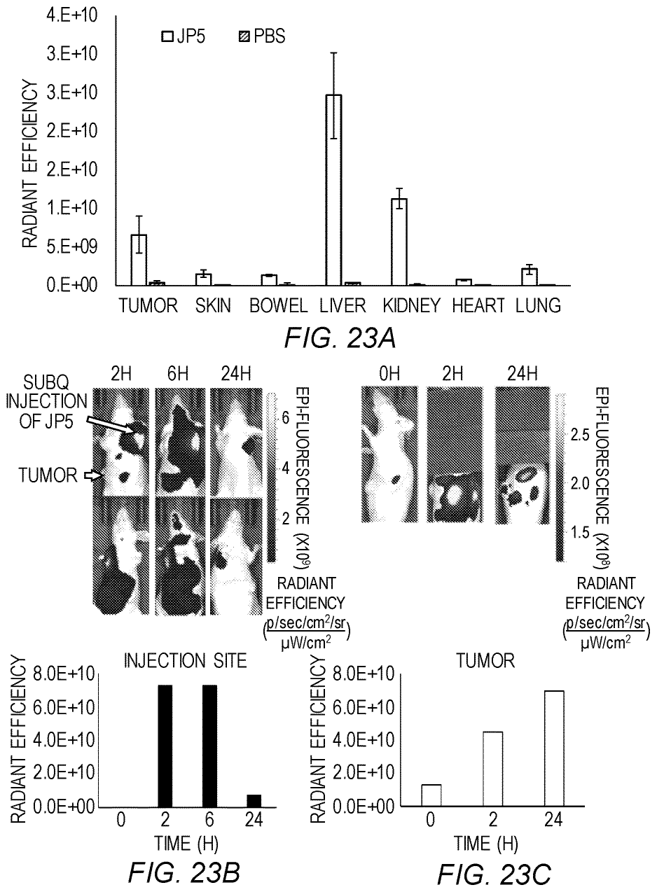

JP5 tissue distribution was determined. Mice were inoculated with subcutaneous A431 tumors (overexpressing B7-H3) and subsequently dosed with JP5-Cy5 (15 nmol/mouse, 200 μL subcutaneously). Mice were sacrificed 2 h post injection, and tissues resected for IVIS imaging (to measure fluorescence). The tissue distribution displayed tumor accumulation, as well as accumulation in liver and kidney (FIG. 23A). Tracking of JP5 distribution in vivo over time is shown in FIG. 23B. Mice were dosed subcutaneously with JP5-Cy5 and imaged at 2 h, 6 h, and 24 h post injection. We observed minimal distribution of JP5 from injection site at 2 h and 6 h. By 24 h post injection, we did observe a decrease in signal for JP5 at the injection site. Tracking of JP5 tumor accumulation in vivo over time is shown in FIG. 23C. In mice dosed subcutaneously with JP5-Cy5 and imaged at 2 h and 24 h post injection, we observed increasing levels of tumor accumulation from 21 h to 24 h post injection. For color scale in FIG. 23B, Min=3.00E8, Max=7.00E9. For color scale in FIG. 23C, Min=1.20E8, Max=2.90E8.

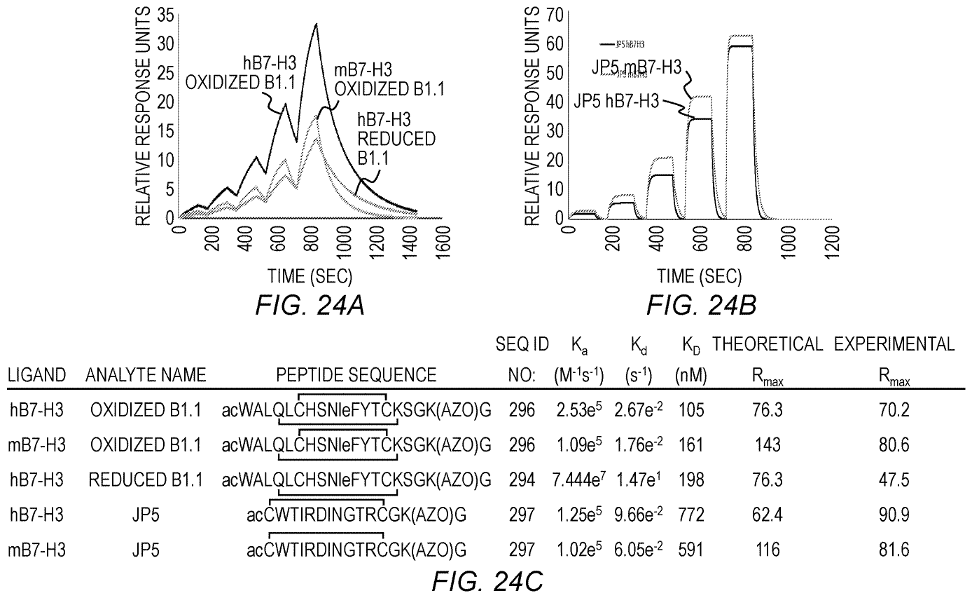

Surface plasmon resonance was used to evaluate binding of B1.1 and JP5 to both human and murine B7-H3 (FIGS. 24A-24C). We evaluated binding of both the oxidized (with disulfide bridge) and reduced (no disulfide bridge) B1.1 against human B7-H3 (hB7-H3)—binding isotherms confirm binding for both species, however the oxidized form of B1.1 displays a stronger binding with a K_{D} of 105 nM vs 198 nM for the reduced form (FIG. 24C). Further, the oxidized B1.1 molecule also binds to murine B7-H3. JP5 binds hB7-H3 with a K_{D} of 772 nM, and mB7-H3 with a K_{D} of 591 nM (FIG. 24C).

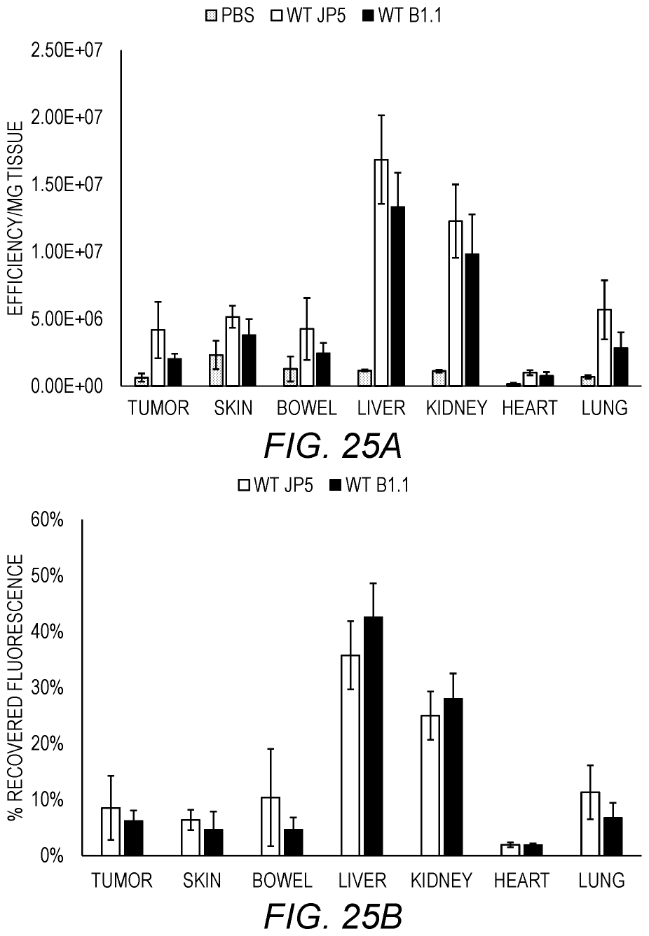

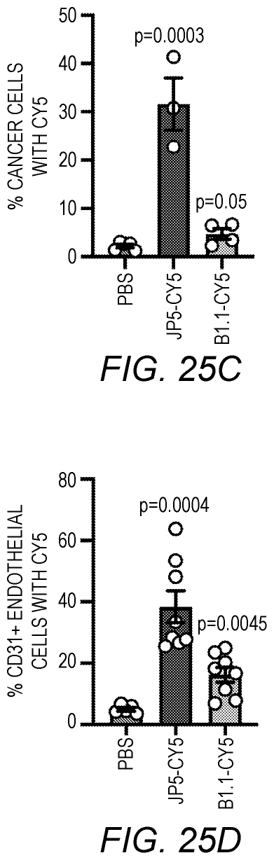

Further analysis demonstrated that both the B1.1 and JP5 ligands (Cy5 labeled) is delivered into tumors. Athymic nude mice bearing A431 wild-type (overexpressing B7H3) flank tumors were treated with PBS, JP5-Cy5, or B1.1-Cy5 (15 nmol, via subcutaneous injection). 24 h post injection, mice were euthanized and tissues harvested and analyzed for fluorescent signal using the IVIS Lumina. Signal for JP5 and B1.1 was observed in the flank tumors, as well as the liver, kidney, and lungs (FIGS. 25A-25B). Tumors were also dissociated into single cells and stained for anti-human HLA to identify tumor cells, and anti-murine CD31 to identify tumor endothelial cells. Both JP5-Cy5 and B1.1-Cy5 was found to significant enrich in cancer cells and tumor endothelial cells, both of which are known to express B7H3 (FIGS. 25C-25D).

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A B7-H3 targeting ligand peptide comprising an amino acid sequence selected from:
a sequence of formula I: 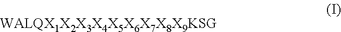
wherein each of X₁-X₉ is any amino acid;
or a sequence of formula II: 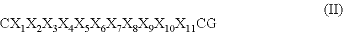
wherein each of X₁-X₉ is any amino acid and X₁₀ and X₁₁ are each independently any amino acid or absent;
wherein each specified amino acid encompasses an amino acid analog or mimetic thereof.

2. The targeting ligand of claim 1, according to formula I, wherein
X₁ is L or an amino acid analog or mimetic thereof,
X₂ is C or an amino acid analog or mimetic thereof,
X₃, X₄ and X₅ are any amino acid,
X₆ is F or an amino acid analog or mimetic thereof,
X₇ is Y or an amino acid analog or mimetic thereof,
X₈ is T or an amino acid analog or mimetic thereof, and
X₉ is C or an amino acid analog or mimetic thereof.

3. The targeting ligand of claim 1, according to formula I, wherein
X₁ is L or an amino acid analog or mimetic thereof,
X₂ is C or an amino acid analog or mimetic thereof,
X₃ is selected from H, V, T, S, and I or an amino acid analog or mimetic of any of these amino acids,
X₄ is S or an amino acid analog or mimetic thereof,
X₅ is selected from M, K, R, Nle, and Y or an amino acid analog or mimetic of any of these amino acids,
X₆ is selected from F, Y, and W or an amino acid analog or mimetic of any of these amino acids,
X₇ is Y or an amino acid analog or mimetic thereof,
X₈ is T or an amino acid analog or mimetic thereof, and
X₉ is C or an amino acid analog or mimetic thereof.

4. The targeting ligand of claim 3, comprising an amino acid sequence selected from:
WALQLCHSMFYTCKSG (SEQ ID NO:2), WALQLCHSNleFYTCKSG (SEQ ID NO:3), WALQLCVSKYYTCKSG (SEQ ID NO:4), WALQLCTSMFYTCKSG (SEQ ID NO:5), WALQLCSSRFYTCKSG (SEQ ID NO:6), WALQLCISRYYTCKSG (SEQ ID NO:7), WALQLCTSYWYTCKSG (SEQ ID NO:8), and WALQLCTSNleFYTCKSG (SEQ ID NO:9), or a sequence at least 80% identical thereto.

5. The targeting ligand according to claim 1, wherein the targeting ligand is according to formula I, comprising an isopeptide bond between a lysine and a glutamine and/or a disulfide bridge between two cysteines in the targeting ligand.

6. The targeting ligand of claim 5, wherein the targeting ligand is N-terminal acetylated WALQLCHSMFYTCKSG (SEQ ID NO:10) or N-terminal acetylated WALQLCHSNleFYTCKSG (SEQ ID NO:11), or a sequence at least 80% identical thereto.

7. The targeting ligand of claim 6, selected from compounds 1 and 2

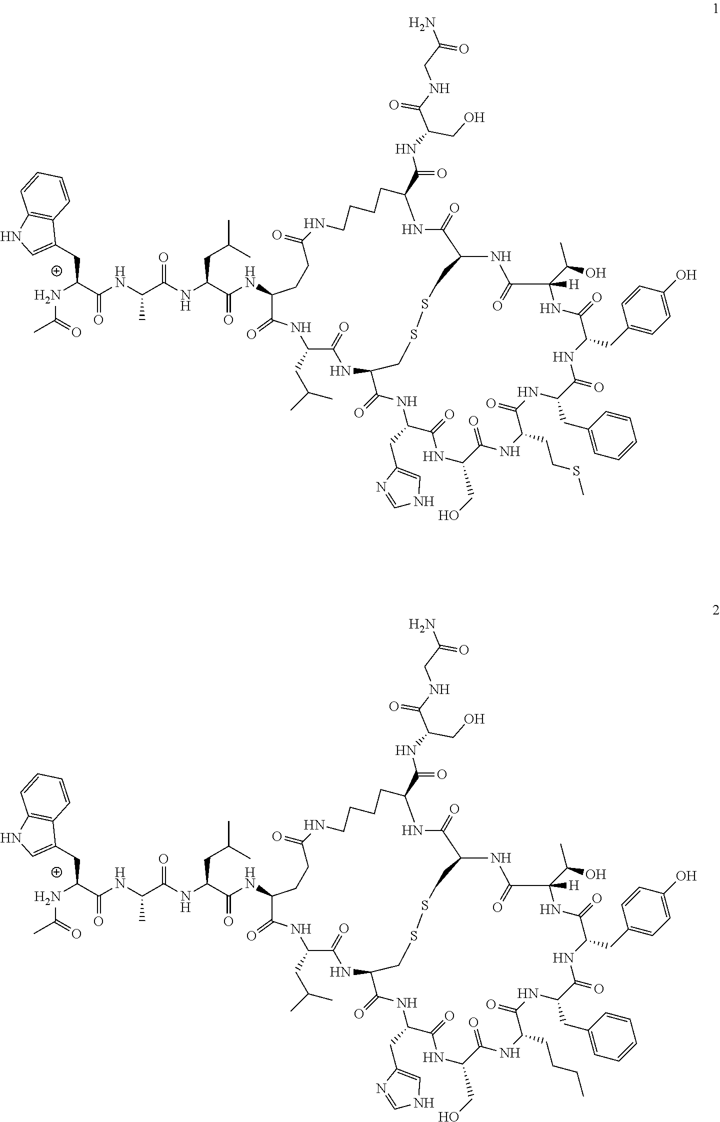

8. The targeting ligand of claim 1, selected from WALQAFTHSCIITKSG (SEQ ID NO:12), WALQYTTHSCIRPKSG (SEQ ID NO:13), and WALQYEIHSCYRNKSG (SEQ ID NO:14), or a sequence at least 80% identical thereto.

9. The targeting ligand of claim 1, according to formula II, wherein
X₁ is selected from V, I, F, and W or an amino acid analog or mimetic of any of these amino acids,
X₂ is selected from V, T, I, K, V, and L or an amino acid analog or mimetic of any of these amino acids,
X₃ is selected from W, I, R, F, V, and Y or an amino acid analog or mimetic of any of these amino acids,
X₄ is selected from T, R, D, W, N, T, and K or an amino acid analog or mimetic of any of these amino acids,
X₅ is selected from R, D, T, D, A, N, R, and G or an amino acid analog or mimetic of any of these amino acids,
X₆ is selected from I, Y, D, L, P, I, R, and G or an amino acid analog or mimetic of any of these amino acids,
X₇ is selected from P, N, Y, A, I, F, P, F, and K or an amino acid analog or mimetic of any of these amino acids,
X₈ is selected from R, G, T, P, N, S, L, and A or an amino acid analog or mimetic of any of these amino acids,
X₉ is selected from F, T, R, Y, I, and L or an amino acid analog or mimetic of any of these amino acids,
X₁₀ is absent, L, R, T, V, W, Y, or I or an amino acid analog or mimetic of any of these amino acids, and
X₁₁ is absent, P, L, or V or an amino acid analog or mimetic of any of these amino acids, or a sequence at least 80% identical thereto.

10. The targeting ligand of claim 9, comprising an amino acid sequence selected from:
CIVWTRIPRFLPCG (SEQ ID NO:15), CWTTRDINGTRCG (SEQ ID NO:16), CVIRDTYYTFTCG (SEQ ID NO:17), CVIFWDDAPRVLCG (SEQ ID NO:18), CVKVNALINYWVCG (SEQ ID NO:19), CVVITNPFSIYCG (SEQ ID NO:20), CFLYKGRFLLCG (SEQ ID NO:21), and CILWNRGKAYICG (SEQ ID NO:22), or a sequence at least 80% identical thereto.

11. The targeting ligand of claim 10, selected from N-terminal acetylated CIVWTRIPRFLPCG (SEQ ID NO:23) and N-terminal acetylated CWTIRDINGTRCG (SEQ ID NO:24), or a sequence at least 80% identical thereto, comprising a thioether bond between the two cysteines of the targeting ligand.

12. The targeting ligand of claim 11, selected from compounds 3 and 4

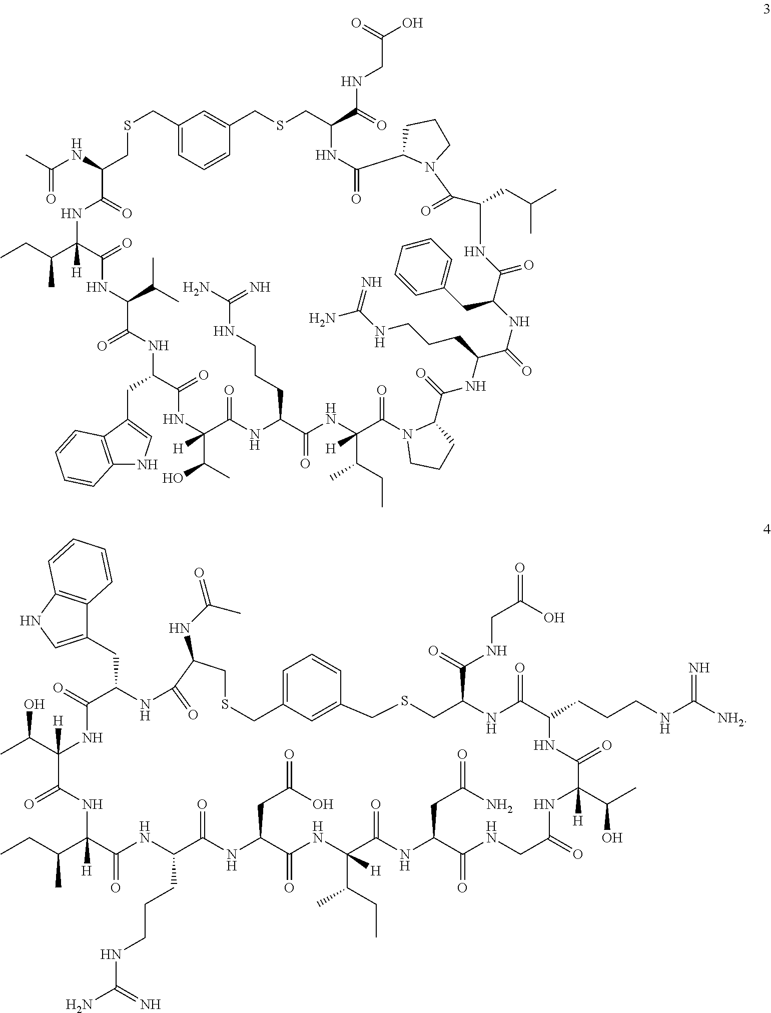

13. The targeting ligand of claim 1, wherein the N-terminus is capped, optionally acetylated.

14. The targeting ligand according to claim 1, further comprising a cargo associated with the ligand.

15. The targeting ligand of claim 14, wherein the cargo is associated by a covalent bond at the N terminus or C terminus of the targeting ligand and/or wherein the cargo is associated with the targeting ligand via a cleavable linker.

16. (canceled)

17. The targeting ligand of claim 14, wherein
the cargo comprises a detectable label, biologically active agent, imaging agent, and/or therapeutic agent, or
wherein the cargo comprises a nucleic acid, a protein, a complex of a nucleic acid and a protein, a carbohydrate, a lipid, or a small molecule.

18-19. (canceled)

20. A pharmaceutical composition comprising the ligand according to claim 1, and a pharmaceutically acceptable carrier.

21. A method of targeting a cargo to a cell that comprises one or more B7-H3 receptors, comprising
contacting the cell with the B7-H3 targeting ligand according to claim 14.

22-24. (canceled)

25. A method of treating a disease or disorder involving a cell comprising B7-H3 in a subject in need thereof, the method comprising administering to the subject a therapeutically effective amount of a targeting ligand according to claim 14.

26. (canceled)

27. A method of delivering a targeting ligand to a cell comprising B7-H3 in a subject, the method comprising administering to the subject the targeting ligand according to claim 1.

28-29. (canceled)


SEQUENCE LISTING (297 sequences)

SEQ ID NO: 1 - protein, 16 aa, synthetic construct
WALQXXXXXXXXXKSG

SEQ ID NO: 2 - protein, 16 aa, synthetic construct
WALQLCHSMFYTCKSG

SEQ ID NO: 3 - protein, 16 aa, synthetic construct
  SITE 9: note = Norleucine
WALQLCHSXFYTCKSG

SEQ ID NO: 4 - protein, 16 aa, synthetic construct
WALQLCVSKYYTCKSG

SEQ ID NO: 5 - protein, 16 aa, synthetic construct
WALQLCTSMFYTCKSG

SEQ ID NO: 6 - protein, 16 aa, synthetic construct
WALQLCSSRFYTCKSG

SEQ ID NO: 7 - protein, 16 aa, synthetic construct
WALQLCISRYYTCKSG

SEQ ID NO: 8 - protein, 16 aa, synthetic construct
WALQLCTSYWYTCKSG

SEQ ID NO: 9 - protein, 16 aa, synthetic construct
  SITE 9: note = Norleucine
WALQLCTSXFYTCKSG

SEQ ID NO: 10 - protein, 16 aa, synthetic construct
  SITE 1: note = Acetylation
WALQLCHSMFYTCKSG

SEQ ID NO: 11 - protein, 16 aa, synthetic construct
  SITE 1: note = Acetylation
  SITE 9: note = Norleucine
WALQLCHSXFYTCKSG

SEQ ID NO: 12 - protein, 16 aa, synthetic construct
WALQAFTHSCIITKSG

SEQ ID NO: 13 - protein, 16 aa, synthetic construct
WALQYTTHSCIRPKSG

SEQ ID NO: 14 - protein, 16 aa, synthetic construct
WALQYEIHSCYRNKSG

SEQ ID NO: 15 - protein, 14 aa, synthetic construct
CIVWTRIPRFLPCG

SEQ ID NO: 16 - protein, 13 aa, synthetic construct
CWTIRDINGTRCG

SEQ ID NO: 17 - protein, 13 aa, synthetic construct
CVIRDTYYTFTCG

SEQ ID NO: 18 - protein, 14 aa, synthetic construct
CVIFWDDAPRVLCG

SEQ ID NO: 19 - protein, 14 aa, synthetic construct
CVKVNALINYWVCG

SEQ ID NO: 20 - protein, 13 aa, synthetic construct
CVVITNPFSIYCG

SEQ ID NO: 21 - protein, 12 aa, synthetic construct
CFLYKGRFLLCG

SEQ ID NO: 22 - protein, 13 aa, synthetic construct
CILWNRGKAYICG

SEQ ID NO: 23 - protein, 14 aa, synthetic construct
  SITE 1: note = Acetylation
CIVWTRIPRFLPCG

SEQ ID NO: 24 - protein, 13 aa, synthetic construct
  SITE 1: note = Acetylation
CWTIRDINGTRCG

SEQ ID NO: 25 - protein, 16 aa, synthetic construct
  SITE 1: note = Acetylation
WALQLCXXXFYTCKSG

SEQ ID NO: 26 - protein, 14 aa, synthetic construct
MWALQRPHGGGKSG

SEQ ID NO: 27 - DNA, 310 bp, synthetic construct
  source 1..310: mol_type = other DNA
cacttcgggctcatgagcgcttgtttcggcgaaattaatacgactcactataggggaattgtgagcggataacaattcccctctagaaataattttgtttaactttaagaaggagatatacatatgtgggcgttgcaacgtccgcatggcggcggtaaaagttaatagcgcattggaagtggataacggatccgaattcgagctccgtcgacaagcttgcggccgcactcgagtgagatccggctgctaacaaagcccgaaaggaagctgagttggctgctgccaccgctgagcaataactagcataacc

SEQ ID NO: 28 - protein, 34 aa, synthetic construct
MWALQRPHGGGKSGSGSGSYPYDVPDYAGSGSGS

SEQ ID NO: 29 - DNA, 165 bp, synthetic construct
  source 1..165: mol_type = other DNA
taatacgactcactatagggttaactttaagaaggagatatacatatgtgggctttacagcgtccacacggaggaggaaaatcgggttcaggtagcggaagttacccgtacgatgttcctgactatgccggcagtgggtcgggatcctaggacggggggcggaaa

SEQ ID NO: 30 - protein, 29 aa, synthetic construct
MXXXQXXXGSGSGSYPYDVPDYAGSGSGS

SEQ ID NO: 31 - DNA, 153 bp, synthetic construct
  source 1..153: mol_type = other DNA
taatacgactcactatagggttaactttaagaaggagatataaatatgnnknnknnkcaannknnknnkagcgggagtggcagcggcagctacccatacgacgtgcccgactatgcaggttctggttctggttcttaggacggggggcggaaa

SEQ ID NO: 32 - protein, 32 aa, synthetic construct
MWALQXXXXKSGSGSGSYPYDVPDYAGSGSGS

SEQ ID NO: 33 - DNA, 159 bp, synthetic construct
  source 1..159: mol_type = other DNA
taatacgactcactatagggttaactttaagaaggagatataaatatgtgggctttacaannknnknnknnkaagagcgggagtggcagcggcagctacccatacgacgtgcccgactatgcaggttctggttctggttcttaggacggggggcggaaa

SEQ ID NO: 34 - protein, 33 aa, synthetic construct
MWLQXXXXXXKSGSGSGSYPYDVPDYAGSGSGS

SEQ ID NO: 35 - DNA, 164 bp, synthetic construct
  source 1..164: mol_type = other DNA
taaacgactcactatagggttaactttaagaaggagatataaatatgtgggctttacaannknnknnknnknnknnkaagagcgggagtggcagcggcagctacccatacgacgtgcccgactatgcaggttctggttctggttcttaggacggggggcggaaa

SEQ ID NO: 36 - protein, 37 aa, synthetic construct
MWALQXXXXXXXXXKSGSGSGSYPYDVPDYAGSGSGS

SEQ ID NO: 37 - DNA, 174 bp, synthetic construct
  source 1..174: mol_type = other DNA
taatacgactcactatagggttaactttaagaaggagatataaatatgtgggctttacaannknnknnknnknnknnknnknnknnkaagagcgggagtggcagcggcagctacccatacgacgtgcccgactatgcaggttctggttctggttcttaggacggggggcggaaa

SEQ ID NO: 38 - DNA, 26 bp, synthetic construct
  source 1..26: mol_type = other DNA
gaaattaatacgactcactatagggg

SEQ ID NO: 39 - DNA, 19 bp, synthetic construct
  source 1..19: mol_type = other DNA
gctagttattgctcagcgg

SEQ ID NO: 40 - DNA, 46 bp, synthetic construct
  source 1..46: mol_type = other DNA
taatacgactcactatagggttaactttaagaaggagatataaata

SEQ ID NO: 41 - DNA, 75 bp, synthetic construct
  source 1..75: mol_type = other DNA
tttccgccccccgtcctaagaaccagaaccagaacctgcatagtcgggcacgtcgtatgggtagctgccgctgcc

SEQ ID NO: 42 - DNA, 91 bp, synthetic construct
  source 1..91: mol_type = other DNA
gtcgtatgggtagctgccgctgccactcccgctcttmnnmnnmnnmnnttgtaaagcccacatatttatatctccttcttaaagttaaccc

SEQ ID NO: 43 - DNA, 97 bp, synthetic construct
  source 1..97: mol_type = other DNA
gtcgtatgggtagctgccgctgccactcccgctcttmnnmnnmnnmnnmnnmnnttgtaaagcccacatatttatatctccttcttaaagttaaccc

SEQ ID NO: 44 - DNA, 106 bp, synthetic construct
  source 1..106: mol_type = other DNA
gtcgtatgggtagctgccgctgccactcccgctcttmnnmnnmnnmnnmnnmnnmnnmnnmnnttgtaaagcccacatatttatatctccttcttaaagttaaccc

SEQ ID NO: 45 - DNA, 85 bp, synthetic construct
  source 1..85: mol_type = other DNA
gtcgtatgggtagctgccgctgccactcccgctmnnmnnmnnttgmnnmnnmnncatatttatatctccttcttaaagttaaccc

SEQ ID NO: 46 - DNA, 39 bp, synthetic construct
  source 1..39: mol_type = other DNA
tttccgccccccgtcctaagaaccagaaccagaacctgc

SEQ ID NO: 47 - DNA, 28 bp, synthetic construct
  source 1..28: mol_type = other DNA
gaaggagatataaatatgtgggctttac

SEQ ID NO: 48 - DNA, 22 bp, synthetic construct
  source 1..22: mol_type = other DNA
cctaagaaccagaaccagaacc

SEQ ID NO: 49 - DNA, 58 bp, synthetic construct
  source 1..58: mol_type = other DNA
acactctttccctacacgacgctcttccgatcttaatacgactcactatagggttaac

SEQ ID NO: 50 - DNA, 58 bp, synthetic construct
  source 1..58: mol_type = other DNA
gactggagttcagacgtgtgctcttccgatcttttccgccccccgtcctaagaaccag

SEQ ID NO: 51 - DNA, 22 bp, synthetic construct
  source 1..22: mol_type = other DNA
tttccgccccccgtcctagctg

SEQ ID NO: 52 - DNA, 16 bp, synthetic construct
  source 1..16: mol_type = other DNA
  misc_feature 16: note = Phosphoramidite 18 spacer
ctcccgccccccgtcc

SEQ ID NO: 53 - protein, 17 aa, synthetic construct
  DISULFID 7..14: note = Disulfide bond
  SITE 1: note = Acetylation
  CROSSLNK 5..15: note = Lactam bridge
MWALQLCHSMFYTCKSG

SEQ ID NO: 54 - protein, 16 aa, synthetic construct
  SITE 1: note = Acetylation
  DISULFID 6..13: note = Disulfide
  CROSSLNK 4..14: note = Lactam bridge
WALQLCHSMFYTCKSG

SEQ ID NO: 55 - protein, 16 aa, synthetic construct
  SITE 1: note = Acetylation
  SITE 9: note = Norleucine
  DISULFID 6..13: note = Disulfide
  CROSSLNK 4..14: note = Lactam bridge
WALQLCHSXFYTCKSG

SEQ ID NO: 56 - protein, 17 aa, synthetic construct
  SITE 1: note = Acetylation
  DISULFID 7..14: note = Disulfide
  CROSSLNK 5..10: note = Lactam bridge
MWALQLCVSKYYTCKSG

SEQ ID NO: 57 - protein, 16 aa, synthetic construct
  SITE 1: note = Acetylation
  DISULFID 6..13: note = Disulfide
  CROSSLNK 4..9: note = Lactam bridge
WALQLCVSKYYTCKSG

SEQ ID NO: 58 - protein, 17 aa, synthetic construct
  SITE 1: note = Acetylation
  DISULFID 7..14: note = Disulfide
  CROSSLNK 5..15: note = Lactam bridge
MWALQLCVSKYYTCKSG

SEQ ID NO: 59 - protein, 16 aa, synthetic construct
  SITE 1: note = Acetylation
  DISULFID 6..13: note = Disulfide
  CROSSLNK 4..14: note = Lactam bridge
WALQLCVSKYYTCKSG

SEQ ID NO: 60 - protein, 17 aa, synthetic construct
  SITE 1: note = Acetylation
  DISULFID 7..14: note = Disulfide
  CROSSLNK 5..15: note = Lactam bridge
MWALQLCTSMFYTCKSG

SEQ ID NO: 61 - protein, 16 aa, synthetic construct
  SITE 1: note = Acetylation
  DISULFID 6..13: note = Disulfide
  CROSSLNK 4..14: note = Lactam bridge
WALQLCTSMFYTCKSG

SEQ ID NO: 62 - protein, 17 aa, synthetic construct
  SITE 1: note = Acetylation
  DISULFID 7..14: note = Disulfide
  CROSSLNK 5..15: note = Lactam bridge
MWALQLCSSRFYTCKSG

SEQ ID NO: 63 - protein, 16 aa, synthetic construct
  SITE 1: note = Acetylation
  DISULFID 6..13: note = Disulfide
  CROSSLNK 4..14: note = Lactam bridge
WALQLCSSRFYTCKSG

SEQ ID NO: 64 - protein, 17 aa, synthetic construct
  SITE 1: note = Acetylation
  DISULFID 7..14: note = Disulfide
  CROSSLNK 5..15: note = Lactam bridge
MWALQLCISRYYTCKSG

SEQ ID NO: 65 - protein, 16 aa, synthetic construct
  SITE 1: note = Acetylation
  DISULFID 6..13: note = Disulfide
  CROSSLNK 4..14: note = Lactam bridge
WALQLCISRYYTCKSG

SEQ ID NO: 66 - protein, 17 aa, synthetic construct
  SITE 1: note = Acetylation
  DISULFID 7..14: note = Disulfide
  CROSSLNK 5..15: note = Lactam bridge
MWALQLCTSYWYTCKSG

SEQ ID NO: 67 - protein, 16 aa, synthetic construct
  SITE 1: note = Acetylation
  DISULFID 6..13: note = Disulfide
  CROSSLNK 4..14: note = Lactam bridge
WALQLCTSYWYTCKSG

SEQ ID NO: 68 - protein, 17 aa, synthetic construct
  SITE 1: note = Acetylation
MWALQAFTHSCIITKSG

SEQ ID NO: 69 - protein, 16 aa, synthetic construct
  SITE 1: note = Acetylation
WALQAFTHSCIITKSG

SEQ ID NO: 70 - protein, 17 aa, synthetic construct
  SITE 1: note = Acetylation
MWALQYTTHSCIRPKSG

SEQ ID NO: 71 - protein, 16 aa, synthetic construct
  SITE 1: note = Acetylation
WALQYTTHSCIRPKSG

SEQ ID NO: 72 - protein, 17 aa, synthetic construct
  SITE 1: note = Acetylation
MWALQYEIHSCYRNKSG

SEQ ID NO: 73 - protein, 16 aa, synthetic construct
  SITE 1: note = Acetylation
WALQYEIHSCYRNKSG

SEQ ID NO: 74 - protein, 8 aa, synthetic construct
MWALQXXX

SEQ ID NO: 75 - protein, 12 aa, synthetic construct
MWALQRPHGGGK

SEQ ID NO: 76 - protein, 10 aa, synthetic construct
MWALQXXXXK

SEQ ID NO: 77 - protein, 12 aa, synthetic construct
MWALQXXXXXXK

SEQ ID NO: 78 - protein, 13 aa, synthetic construct
  SITE 1: note = Acetylation
  CROSSLNK 5..9: note = Lactam bridge
MWVLQYAHKLIKS

SEQ ID NO: 79 - protein, 37 aa, synthetic construct
MWALQLCHSMFYTCKSGSGSGSYPYDVPDYAGSGSGS

SEQ ID NO: 80 - protein, 37 aa, synthetic construct
MWALQLCHSMFYTCKIGSGSGSYPYDVPDYAGSGSGS

SEQ ID NO: 81 - protein, 37 aa, synthetic construct
MWALQLCHSMFYTCKSGSGSGSYPYDVSDYAGSGSGS

SEQ ID NO: 82 - protein, 37 aa, synthetic construct
MWALQLCHSMFYTCKSGSGSGSYPYDVPDYAGSGSGF

SEQ ID NO: 83 - protein, 37 aa, synthetic construct
MWALQLCTSMFYTCKSGSGSGSYPYDVPDYAGSGSGS

SEQ ID NO: 84 - protein, 37 aa, synthetic construct
MWALQLCSSRFYTCKSGSGSGSYPYDVPDYAGSGSGS

SEQ ID NO: 85 - protein, 37 aa, synthetic construct
MWALQLCVSMFYTCKSGSGSGSYPYDVPDYAGSGSGS

SEQ ID NO: 86 - protein, 37 aa, synthetic construct
MWALQLCVSKFYTCKSGSGSGSYPYDVPDYAGSGSGS

SEQ ID NO: 87 - protein, 37 aa, synthetic construct
MWALQLCHSMFYTCKSGSGSGSYPYDVPDFAGSGSGS

SEQ ID NO: 88 - protein, 37 aa, synthetic construct
MWALQLCHSKYYTCKSGSGSGSYPYDVPDYAGSGSGS

SEQ ID NO: 89 - protein, 37 aa, synthetic construct
MWALQLCHSMYYTCKSGSGSGSYPYDVPDYAGSGSGS

SEQ ID NO: 90 - protein, 37 aa, synthetic construct
MWALQLCHSMFYTCNSGSGSGSYPYDVPDYAGSGSGS

SEQ ID NO: 91 - protein, 37 aa, synthetic construct
MWALQLCQSMFYTCKSGSGSGSYPYDVPDYAGSGSGS

SEQ ID NO: 92 - protein, 37 aa, synthetic construct
MWALQLCVSKYYTCKSGSGSGSYPYDVPDYAGSGSGS

SEQ ID NO: 93 - protein, 37 aa, synthetic construct
MWALQLCISRYYTCKSGSGSGSYPYDVPDYAGSGSGS

SEQ ID NO: 94 - protein, 37 aa, synthetic construct
MWALQLCVSKYYTCNSGSGSGSYPYDVPDYAGSGSGS

SEQ ID NO: 95 - protein, 37 aa, synthetic construct
MWALQLCTSYWYTCKSGSGSGSYPYDVPDYAGSGSGS

SEQ ID NO: 96 - protein, 29 aa, synthetic construct
MWALQLCHSMFYTCKSGSGSGSYPYDVPD

SEQ ID NO: 97 - protein, 29 aa, synthetic construct
MWALQLCVSKYYTCKSGSGSGSYPYDVPD

SEQ ID NO: 98 - protein, 37 aa, synthetic construct
MWALQDRPWIYNKNKSGSGSGSYPYDVPDYAGSGSGS

SEQ ID NO: 99 - protein, 16 aa, synthetic construct
MWALQLCHSMFYTCKS

SEQ ID NO: 100 - protein, 16 aa, synthetic construct
MWALQLCVSKYYTCKS

SEQ ID NO: 101 - protein, 16 aa, synthetic construct
MWALQLCTSMFYTCKS

SEQ ID NO: 102 - protein, 16 aa, synthetic construct
MWALQLCSSRFYTCKS

SEQ ID NO: 103 - protein, 16 aa, synthetic construct
MWALQLCISRYYTCKS

SEQ ID NO: 104 - protein, 16 aa, synthetic construct
MWALQLCTSYWYTCKS

SEQ ID NO: 105 - protein, 7 aa, synthetic construct
MWALQKS

SEQ ID NO: 106 - protein, 16 aa, synthetic construct
  SITE 1: note = Acetylation
WALQLCHSMFYTCKSG

SEQ ID NO: 107 - protein, 36 aa, synthetic construct
  CROSSLNK 5..15: note = Lactam bridge
  VARIANT 6..8: note = Amino acids are present or absent
MWALQXXXXXXXXXKGSGSGSYPYDVPDYAGSGSGS

SEQ ID NO: 108 - protein, 35 aa, synthetic construct
  DISULFID 2..14: note = Disulfide
  VARIANT 3..4: note = Amino acids are present or absent
MCXXXXXXXXXXXCGSGSGSYPYDVPDYAGSGSGS

SEQ ID NO: 109 - protein, 17 aa, synthetic construct
MWALQXXXXXXXXXKSG

SEQ ID NO: 110 - protein, 11 aa, synthetic construct
QATFHSCIITK

SEQ ID NO: 111 - protein, 11 aa, synthetic construct
QYTTHASIRPK

SEQ ID NO: 112 - protein, 11 aa, synthetic construct
QYEIHSCYRNK

SEQ ID NO: 113 - protein, 6 aa, synthetic construct
MWALQK

SEQ ID NO: 114 - protein, 14 aa, synthetic construct
MCIVWTRIPRFLPC

SEQ ID NO: 115 - protein, 14 aa, synthetic construct
MCIVWTRTPRFVRC

SEQ ID NO: 116 - protein, 14 aa, synthetic construct
MCIVFTDRARFVRC

SEQ ID NO: 117 - protein, 13 aa, synthetic construct
MCILWNRGKAYIC

SEQ ID NO: 118 - protein, 14 aa, synthetic construct
MCIIWSPHASLYRC

SEQ ID NO: 119 - protein, 14 aa, synthetic construct
MCIIFAPYARLYRC

SEQ ID NO: 120 - protein, 13 aa, synthetic construct
MCIKYANGNWFLC

SEQ ID NO: 121 - protein, 13 aa, synthetic construct
MCITIWKSKYILC

SEQ ID NO: 122 - protein, 13 aa, synthetic construct
MCIKMYRGNYFIC

SEQ ID NO: 123 - protein, 13 aa, synthetic construct
MCIYWVNNRPHLC

SEQ ID NO: 124 - protein, 14 aa, synthetic construct
MCIKYVNNVGFVMC

SEQ ID NO: 125 - protein, 12 aa, synthetic construct
MCIQWGWPRTWC

SEQ ID NO: 126 - protein, 14 aa, synthetic construct
MCIVWIPKPTFVYC

SEQ ID NO: 127 - protein, 12 aa, synthetic construct
MCITWDGYLINC

SEQ ID NO: 128 - protein, 12 aa, synthetic construct
MCITWDNYLVHC

SEQ ID NO: 129 - protein, 14 aa, synthetic construct
MCIVWLPTPHFYMC

SEQ ID NO: 130 - protein, 14 aa, synthetic construct
MCIVWNTHPTLWHC

SEQ ID NO: 131 - protein, 12 aa, synthetic construct
MCNCYLWHEMFC

SEQ ID NO: 132 - protein, 13 aa, synthetic construct
MCFCTYLYNTMFC

SEQ ID NO: 133 - protein, 13 aa, synthetic construct
MCLKYHSGKWFLC

SEQ ID NO: 134 - protein, 13 aa, synthetic construct
MCFYNDRGTVYLC

SEQ ID NO: 135 - protein, 14 aa, synthetic construct
MCLRYLPHHGYLIC

SEQ ID NO: 136 - protein, 12 aa, synthetic construct
MCYIFNASTLWC

SEQ ID NO: 137 - protein, 13 aa, synthetic construct
MCWTIRDINGTRC

SEQ ID NO: 138 - protein, 13 aa, synthetic construct
MCVIRDTYYTFTC

SEQ ID NO: 139 - protein, 14 aa, synthetic construct
MCVIFWDDAPRVLC

SEQ ID NO: 140 - protein, 13 aa, synthetic construct
MCYLYKARFLWPC

SEQ ID NO: 141 - protein, 12 aa, synthetic construct
CWTIRDINGTRC

SEQ ID NO: 142 - protein, 12 aa, synthetic construct
CVIRDTYYTFTC

SEQ ID NO: 143 - protein, 13 aa, synthetic construct
CVIFWDDAPRVLC

SEQ ID NO: 144 - protein, 13 aa, synthetic construct
CVKVNALINYWVC

SEQ ID NO: 145 - protein, 12 aa, synthetic construct
CVVITNPFSIYC

SEQ ID NO: 146 - protein, 13 aa, synthetic construct
CVWPGQTFYTIVC

SEQ ID NO: 147 - protein, 11 aa, synthetic construct
CVIYKGKILPC

SEQ ID NO: 148 - protein, 13 aa, synthetic construct
CIVWTRIPRFLPC

SEQ ID NO: 149 - protein, 11 aa, synthetic construct
CFLYKGRFLLC

SEQ ID NO: 150 - protein, 12 aa, synthetic construct
CILWNRGKAYIC

SEQ ID NO: 151 - protein, 12 aa, synthetic construct
CIYWVNNRPHLC

SEQ ID NO: 152 - protein, 13 aa, synthetic construct
CIVWLPTPHFYMC

SEQ ID NO: 153 - protein, 11 aa, synthetic construct
CIFYNTYWIKC

SEQ ID NO: 154 - protein, 12 aa, synthetic construct
CIKYANGNWFLC

SEQ ID NO: 155 - protein, 11 aa, synthetic construct
CIQWGWPRTWC

SEQ ID NO: 156 - protein, 12 aa, synthetic construct
CIKMYRGNYFIC

SEQ ID NO: 157 - protein, 11 aa, synthetic construct
CITWDGYLINC

SEQ ID NO: 158 - protein, 13 aa, synthetic construct
CIVWKERPYLIVC

SEQ ID NO: 159 - protein, 11 aa, synthetic construct
CITWDNYLVHC

SEQ ID NO: 160 - protein, 12 aa, synthetic construct
CLKYHSGKWFLC

SEQ ID NO: 161 - protein, 13 aa, synthetic construct
CLRYLPHHGYLIC

SEQ ID NO: 162 - protein, 11 aa, synthetic construct
CNCYLWHEMFC

SEQ ID NO: 163 - protein, 11 aa, synthetic construct
CYIFNASTLWC

SEQ ID NO: 164 - protein, 14 aa, synthetic construct
  SITE 1: note = Acetylation
  DISULFID 1..13: note = Disulfide
CIVWTRIPRFLPCG

SEQ ID NO: 165 - protein, 13 aa, synthetic construct
  SITE 1: note = Acetylation
  DISULFID 1..12: note = Disulfide
CWTIRDINGTRCG

SEQ ID NO: 166 - protein, 16 aa, synthetic construct
  SITE 1: note = Acetylation
  SITE 9: note = Norleucine
  DISULFID 6..13: note = Disulfide
  CROSSLNK 4..14: note = Lactam bridge
WALQLCTSXFYTCKSG

SEQ ID NO: 167 - protein, 35 aa, synthetic construct
MCIVWTRIPRFLPCGSGSGSYPYDVPDYAGSGSGS

SEQ ID NO: 168 - protein, 11 aa, synthetic construct
IVWTRIPRFLP

SEQ ID NO: 169 - protein, 35 aa, synthetic construct
MCIVWTRIPRFLPCGSGSGSYPYDVPDYVGSGSGS

SEQ ID NO: 170 - protein, 35 aa, synthetic construct
MCIVWTRIPRFLPCGSGSGSYPYDVPDYAGSRSGS

SEQ ID NO: 171 - protein, 35 aa, synthetic construct
MCIVWTYPPRLVPCGSGSGSYPYDVPDYAGSGSGS

SEQ ID NO: 172 - protein, 11 aa, synthetic construct
IVWTYPPRLVP

SEQ ID NO: 173 - protein, 35 aa, synthetic construct
MCIVWTRIPRFLPCGSGSGSYPYDVPDYSGSGSGS

SEQ ID NO: 174 - protein, 35 aa, synthetic construct
MCIVWTRIPRFLPCGSGLGSYPYDVPDYAGSGSGS

SEQ ID NO: 175 - protein, 35 aa, synthetic construct
MCIVWTRIPRFLPCGSGSGSYPYDVPDYARSGSGS

SEQ ID NO: 176 - protein, 35 aa, synthetic construct
MCIVWTRIPRFLPCGSGSVSYPYDVPDYAGSGSGS

SEQ ID NO: 177 - protein, 35 aa, synthetic construct
MCIVWTRIPRFLPCGSGSGLYPYDVPDYAGSGSGS

SEQ ID NO: 178 - protein, 35 aa, synthetic construct
MCIVWTRIPRFLPCGSGSGSYPYDVPDYAWSGSGS

SEQ ID NO: 179 - protein, 34 aa, synthetic construct
MCIYWVNNRPHLCGSGSGSYPYDVPDYAGSGSGS

SEQ ID NO: 180 - protein, 10 aa, synthetic construct
IYWVNNRPHL

SEQ ID NO: 181 - protein, 34 aa, synthetic construct
MCIYWTGDVPRLCGSGSGWYPYDVPDYAGSGSGS

SEQ ID NO: 182 - protein, 10 aa, synthetic construct
IYWTGDVPRL

SEQ ID NO: 183 - protein, 34 aa, synthetic construct
MCIYWTGNRARICGSGSGSYPYDVPDYAGSGSGS

SEQ ID NO: 184 - protein, 10 aa, synthetic construct
IYWTGNRARI

SEQ ID NO: 185 - protein, 34 aa, synthetic construct
MCIYWTSGRARICGSGSGSYPYDVPDYAGSGSGS

SEQ ID NO: 186 - protein, 10 aa, synthetic construct
IYWTSGRARI

SEQ ID NO: 187 - protein, 34 aa, synthetic construct
MCIYWTGDVPRLCGSGSGSYPYDVPDYAGSGSGS

SEQ ID NO: 188 - protein, 34 aa, synthetic construct
MCIYWVNNRPHLCSSGSGSYPYDVPDYAGSGSGS

SEQ ID NO: 189 - protein, 34 aa, synthetic construct
MCIYWVNNRPHLCGSGSGSYPYDVPDYVGSGSGS

SEQ ID NO: 190 - protein, 34 aa, synthetic construct
MCIYWVNNRPHLCGSGSGSYPYDVPDYARSGSGS

SEQ ID NO: 191 - protein, 34 aa, synthetic construct
MCIYWVTNRPFLCGSGSGSYPYDVPDYAGSGSGS

SEQ ID NO: 192 - protein, 10 aa, synthetic construct
IYWVTNRPFL

SEQ ID NO: 193 - protein, 34 aa, synthetic construct
MCIYWVNNRPHLCGSGSGSYPYDVPDYAWSGSGS

SEQ ID NO: 194 - protein, 34 aa, synthetic construct
MCIFIDDLYGMRCGSGSGSYPYDVPDYAGSGSGS

SEQ ID NO: 195 - protein, 10 aa, synthetic construct
IFIDDLYGMR

SEQ ID NO: 196 - protein, 34 aa, synthetic construct
MCWTIRDINGTRCGSGSGSYPYDVPDYAGSGSGS

SEQ ID NO: 197 - protein, 10 aa, synthetic construct
WTIRDINGTR

SEQ ID NO: 198 - protein, 34 aa, synthetic construct
MCVEIWDISGYRCGSGSGSYPYDVPDYAGSGSGS

SEQ ID NO: 199 - protein, 10 aa, synthetic construct
VEIWDISGYR

SEQ ID NO: 200 - protein, 34 aa, synthetic construct
MCIFIDDLYGMRCGSGSGSYPYDVPDYVGSGSGS

SEQ ID NO: 201 - protein, 34 aa, synthetic construct
MCIFIDDLYGMRCGSGSWSYPYDVPDYAGSGSGS

SEQ ID NO: 202 - protein, 34 aa, synthetic construct
MCIFIDDLYWMRCGSGSGSYPYDVPDYAGSGSGS

SEQ ID NO: 203 - protein, 10 aa, synthetic construct
IFIDDLYWMR

SEQ ID NO: 204 - protein, 34 aa, synthetic construct
MCIFIDDLYGMRCGSGSGSYPYDVPDYAGSVSGS

SEQ ID NO: 205 - protein, 34 aa, synthetic construct
MCIFIDDLYGMRCGSGSGSYPYYVPDYAGSGSGS

SEQ ID NO: 206 - protein, 34 aa, synthetic construct
MCIFIDDLYGMRCGSGSVSYPYDVPDYAGSGSGS

SEQ ID NO: 207 - protein, 34 aa, synthetic construct
MCIFIDDLYGMRCGSGSGSYPYDVPDYAWSGSGS

SEQ ID NO: 208 - protein, 34 aa, synthetic construct
MCILPWHGQFLICGSGSGSYPYDVPDYAGSGSGS

SEQ ID NO: 209 - protein, 10 aa, synthetic construct
ILPWHGQFLI

SEQ ID NO: 210 - protein, 34 aa, synthetic construct
MCITFYRGNFVICGSGSGSYPYDVPDYAGSGSGS

SEQ ID NO: 211 - protein, 10 aa, synthetic construct
ITFYRGNFVI

SEQ ID NO: 212 - protein, 34 aa, synthetic construct
MCIYPYRGAFLLCGSGSGSYPYDVPDYAGSGSGS

SEQ ID NO: 213 - protein, 10 aa, synthetic construct
IYPYRGAFLL

SEQ ID NO: 214 - protein, 34 aa, synthetic construct
MCILPTTGGFYLCGSGSGSYPYDVPDYARSGSGS

SEQ ID NO: 215 - protein, 10 aa, synthetic construct
ILPTTGGFYL

SEQ ID NO: 216 - protein, 34 aa, synthetic construct
MCVFPYRGKFFICGSGSGSYPYDVPDYAGSGSGS

SEQ ID NO: 217 - protein, 10 aa, synthetic construct
VFPYRGKFFI

SEQ ID NO: 218 - protein, 34 aa, synthetic construct
MCIRTYYGQWLLCGSGSGSYPYDVPDYAGSGSGS

SEQ ID NO: 219 - protein, 10 aa, synthetic construct
IRTYYGQWLL

SEQ ID NO: 220 - protein, 34 aa, synthetic construct
MCIVPYLGAFLICGSGSGSYPYDVPDYAGSGSGS

SEQ ID NO: 221 - protein, 10 aa, synthetic construct
IVPYLGAFLI

SEQ ID NO: 222 - protein, 34 aa, synthetic construct
MCIKPYRNNFLICGSGSGSYPYDVPDYAGSGSGS

SEQ ID NO: 223 - protein, 10 aa, synthetic construct
IKPYRNNFLI

SEQ ID NO: 224 - protein, 34 aa, synthetic construct
MCILPFNGLYFLCGSGSGSYPYDVPDYAGSGSGS

SEQ ID NO: 225 - protein, 10 aa, synthetic construct
ILPFNGLYFL

SEQ ID NO: 226 - protein, 34 aa, synthetic construct
MCLLQWHGGFYLCGSGSGSYPYDVPDYAGSGSGS

SEQ ID NO: 227 - protein, 10 aa, synthetic construct
LLQWHGGFYL

SEQ ID NO: 228 - protein, 34 aa, synthetic construct
MCIKVIGSRFYLCGSGSGSYPYDVPDYAGSGSGS

SEQ ID NO: 229 - protein, 10 aa, synthetic construct
IKVIGSRFYL

SEQ ID NO: 230 - protein, 34 aa, synthetic construct
MCIRYVGGHFYLCGSGSGSYPYDVPDYAGSGSGS

SEQ ID NO: 231 - protein, 10 aa, synthetic construct
IRYVGGHFYL

SEQ ID NO: 232 - protein, 34 aa, synthetic construct
MCIRLESGKFYLCGSGSGSYPYDVPDYAGSGSGS

SEQ ID NO: 233 - protein, 10 aa, synthetic construct
IRLESGKFYL

SEQ ID NO: 234 - protein, 34 aa, synthetic construct
MCIKYILGSWYLCGSGSGSYPYDVPDYAGSGSGS

SEQ ID NO: 235 - protein, 10 aa, synthetic construct
IKYILGSWYL

SEQ ID NO: 236 - protein, 34 aa, synthetic construct
MCIKVNGPHFYLCGSGLGSYPYDVPDYAGSGSGS

SEQ ID NO: 237 - protein, 10 aa, synthetic construct
IKVNGPHFYL

SEQ ID NO: 238 - protein, 34 aa, synthetic construct
MCIRFTRAGFLLCGSGSGSYPYDVPDYAGSGSGS

SEQ ID NO: 239 - protein, 10 aa, synthetic construct
IRFTRAGFLL

SEQ ID NO: 240 - protein, 34 aa, synthetic construct
MCIRYVLNNWYLCGSGSGSYPYDVPDYAGSGSGS

SEQ ID NO: 241 - protein, 10 aa, synthetic construct
IRYVLNNWYL

SEQ ID NO: 242 - protein, 34 aa, synthetic construct
MCIRFVSGYYYLCGSGSGSYPYDVPDYAGSGSGS

SEQ ID NO: 243 - protein, 10 aa, synthetic construct
IRFVSGYYYL

SEQ ID NO: 244 - protein, 34 aa, synthetic construct
MCIRLMQGHFYLCGSGSGSYPYDVPDYAGSGSGS

SEQ ID NO: 245 - protein, 10 aa, synthetic construct
IRLMQGHFYL

SEQ ID NO: 246 - protein, 34 aa, synthetic construct
MCIALLKGRFYLCGSGSGSYPYDVPDYAGSGSGS

SEQ ID NO: 247 - protein, 10 aa, synthetic construct
IALLKGRFYL

SEQ ID NO: 248 - protein, 34 aa, synthetic construct
MCWKITDVYYVICGSGSGSYPYDVPDYAGSGSGS

SEQ ID NO: 249 - protein, 10 aa, synthetic construct
WKITDVYYVI

SEQ ID NO: 250 - protein, 34 aa, synthetic construct
MCWSIWDIHGVRCGSGSGSYPYDVPDYAGSGSGS

SEQ ID NO: 251 - protein, 10 aa, synthetic construct
WSIWDIHGVR

SEQ ID NO: 252 - protein, 34 aa, synthetic construct
MCIKLLNGYFIICGSGSGSYPYDVPDYAGSGSGS

SEQ ID NO: 253 - protein, 10 aa, synthetic construct
IKLLNGYFII

SEQ ID NO: 254 - protein, 34 aa, synthetic construct
MCWKITDVYYVICGSGSGSYPYDVPDYVGSGSGS

SEQ ID NO: 255 - protein, 34 aa, synthetic construct
MCWKITEVYYVICGSGSGSYPYDVPDYAGSGSGS

SEQ ID NO: 256 - protein, 10 aa, synthetic construct
WKITEVYYVI

SEQ ID NO: 257 - protein, 34 aa, synthetic construct
MCWKITDVYYVICGSGSGSYPYDVPDYARSGSGS

SEQ ID NO: 258 - protein, 34 aa, synthetic construct
MCWKITDVYYVICGSGSGSYPYDVPDYAGRGSGS

SEQ ID NO: 259 - protein, 34 aa, synthetic construct
MCIRFVSVYYYLCGSGSGSYPYDVPDYAGSGSGS

SEQ ID NO: 260 - protein, 10 aa, synthetic construct
IRFVSVYYYL

SEQ ID NO: 261 - protein, 34 aa, synthetic construct; identical to SEQ ID NO: 248

SEQ ID NO: 262 - protein, 34 aa, synthetic construct
MCWKITDVYYVICGNGSGSYPYDVPDYAGSGSGS

SEQ ID NO: 263 - protein, 12 aa, synthetic construct
  SITE 1: note = Acetylation
CXTIRDINGTRC

SEQ ID NO: 264 - protein, 12 aa, synthetic construct
  SITE 1: note = Acetylation
CWXIRDINGTRC

SEQ ID NO: 265 - protein, 12 aa, synthetic construct
  SITE 1: note = Acetylation
CWTXRDINGTRC

SEQ ID NO: 266 - protein, 12 aa, synthetic construct
  SITE 1: note = Acetylation
CWTIXDINGTRC

SEQ ID NO: 267 - protein, 12 aa, synthetic construct
  SITE 1: note = Acetylation
CWTIRXINGTRC

SEQ ID NO: 268 - protein, 12 aa, synthetic construct
  SITE 1: note = Acetylation
CWTIRDXNGTRC

SEQ ID NO: 269 - protein, 12 aa, synthetic construct
  SITE 1: note = Acetylation
CWTIRDIXGTRC

SEQ ID NO: 270 - protein, 12 aa, synthetic construct
  SITE 1: note = Acetylation
CWTIRDINXTRC

SEQ ID NO: 271 - protein, 12 aa, synthetic construct
  SITE 1: note = Acetylation
CWTIRDINGXRC

SEQ ID NO: 272 - protein, 12 aa, synthetic construct
  SITE 1: note = Acetylation
CWTIRDINGTXC

SEQ ID NO: 273 - protein, 13 aa, synthetic construct
  SITE 1: note = Acetylation
CXVWTRIPRFLPC

SEQ ID NO: 274 - protein, 13 aa, synthetic construct
  SITE 1: note = Acetylation
CIXWTRIPRFLPC

SEQ ID NO: 275 - protein, 13 aa, synthetic construct
  SITE 1: note = Acetylation
CIVXTRIPRFLPC

SEQ ID NO: 276 - protein, 13 aa, synthetic construct
  SITE 1: note = Acetylation
CIVWXRIPRFLPC

SEQ ID NO: 277 - protein, 13 aa, synthetic construct
  SITE 1: note = Acetylation
CIVWTXIPRFLPC

SEQ ID NO: 278 - protein, 13 aa, synthetic construct
  SITE 1: note = Acetylation
CIVWTRXPRFLPC

SEQ ID NO: 279 - protein, 13 aa, synthetic construct
  SITE 1: note = Acetylation
CIVWTRIXRFLPC

SEQ ID NO: 280 - protein, 13 aa, synthetic construct
  SITE 1: note = Acetylation
CIVWTRIPXFLPC

SEQ ID NO: 281 - protein, 13 aa, synthetic construct
  SITE 1: note = Acetylation
CIVWTRIPRXLPC

SEQ ID NO: 282 - protein, 13 aa, synthetic construct
  SITE 1: note = Acetylation
CIVWTRIPRFXPC

SEQ ID NO: 283 - protein, 13 aa, synthetic construct
  SITE 1: note = Acetylation
CIVWTRIPRFLXC

SEQ ID NO: 284 - protein, 12 aa, synthetic construct
  SITE 1: note = Acetylation
CATIRDINGTRC

SEQ ID NO: 285 - protein, 12 aa, synthetic construct
  SITE 1: note = Acetylation
CWAIRDINGTRC

SEQ ID NO: 286 - protein, 12 aa, synthetic construct
  SITE 1: note = Acetylation
CWTARDINGTRC

SEQ ID NO: 287 - protein, 12 aa, synthetic construct
  SITE 1: note = Acetylation
CWTIADINGTRC

SEQ ID NO: 288 - protein, 12 aa, synthetic construct
  SITE 1: note = Acetylation
CWTIRAINGTRC

SEQ ID NO: 289 - protein, 12 aa, synthetic construct
  SITE 1: note = Acetylation
CWTIRDANGTRC

SEQ ID NO: 290 - protein, 12 aa, synthetic construct
  SITE 1: note = Acetylation
CWTIRDIAGTRC

SEQ ID NO: 291 - protein, 12 aa, synthetic construct
  SITE 1: note = Acetylation
CWTIRDINATRC

SEQ ID NO: 292 - protein, 12 aa, synthetic construct
  SITE 1: note = Acetylation
CWTIRDINGARC

SEQ ID NO: 293 - protein, 12 aa, synthetic construct
  SITE 1: note = Acetylation
CWTIRDINGTAC

SEQ ID NO: 294 - protein, 18 aa, synthetic construct
  SITE 1: note = Acetylation
  SITE 9: note = Norleucine
  CROSSLNK 4..14: note = Lactam bridge
  SITE 17: note = Azido lysine
WALQLCHSXFYTCKSGXG

SEQ ID NO: 295 - protein, 12 aa, synthetic construct
  DISULFID 1..12: note = Disulfide
  SITE 1: note = Acetylation
CWTIRDINGTRC

SEQ ID NO: 296 - protein, 18 aa, synthetic construct
  SITE 1: note = Acetylation
  SITE 9: note = Norleucine
  SITE 17: note = Azido lysine
  DISULFID 6..13: note = Disulfide
  CROSSLNK 4..14: note = Lactam bridge
WALQLCHSXFYTCKSGXG

SEQ ID NO: 297 - protein, 15 aa, synthetic construct
  SITE 1: note = Acetylation
  DISULFID 1..12: note = Disulfide
  SITE 14: note = Azido lysine
CWTIRDINGTRCGXG